(19) United States
(12) Patent Application Publication
(10) Pub. No.: US 2025/0027123 A1
(43) Pub. Date: Jan. 23, 2025

(54) Compositions, Systems, and Methods for Performing a Mannich Cyclization Reaction

(71) Applicant: The Regents of the University of California, Oakland, CA (US)

(72) Inventors: Yang Hai, Santa Barbara, CA (US); Jinmin Gao, Isla Vista, CA (US); Shaonan Liu, Goleta, CA (US)

(73) Assignee: The Regents of the University of California, Oakland, CA (US)

(21) Appl. No.: 18/766,067

(22) Filed: Jul. 8, 2024

Related U.S. Application Data
(60) Provisional application No. 63/512,236, filed on Jul. 6, 2023.

(51) Int. Cl. C12P 13/04; C12N 1/06; C12N 1/20; C12N 9/10; C12R 1/19

(52) U.S. Cl. CPC C12P 13/04; C12N 1/06; C12N 1/205; C12N 9/1096; C12R 2001/19; C12Y 206/01

(57) ABSTRACT

Compositions, systems and methods for performing Mannich cyclization reaction and applications thereof are described. Various enzymes, which are referred to as Mannich cyclases, are utilized to catalyze Mannich cyclization with various substrates to yield an azacyclic α-amino acid. Organisms can express or produce a Mannich cyclase via heterologous expression. Methods can be performed to produce a larger molecule, including a therapeutic, using a Mannich cyclase to produce a precursor compound for the larger molecule.

### CROSS REFERENCE TO RELATED APPLICATIONS

This application claims priority to U.S. Provisional Patent Application No. 63/512,236 entitled “Compositions, Systems, and Methods for Performing a Mannich Cyclization Reaction,” filed Jul. 6, 2023, the disclosure of which is incorporated herein by reference.

### TECHNICAL FIELD

The disclosure is generally directed to compositions, systems, and methods for performing a Mannich cyclization reaction, and more specifically the disclosure includes description of enzymes for performing Mannich cyclization.

### SEQUENCE LISTING

This application hereby incorporates by reference the material of the electronic Sequence Listing filed concurrently herewith. The material in the electronic Sequence Listing is submitted as an XML (.xml) file entitled “R39-08066 SEQ” created on Jul. 8, 2024, which has a file size of 12 KB, and is herein incorporated by reference in its entirety.

### BACKGROUND

Amino acids (AA) are molecules of life since they play a central role in making proteins and diverse metabolites in nature. They are also essential building blocks in making pharmaceuticals, such as peptides and peptidomimetics. Among all AAs, α,α-disubstituted α-AAs are unique in that they are sterically and conformationally constrained, and have additional advantage of being resistant to racemization and proteolysis when incorporated in peptides. Despite their promising role in pharmaceuticals, the application of α,α-disubstituted α-AAs are limited by inefficient routes of production. Therefore, it is of great importance to develop efficient methods for asymmetric synthesis of different α,α-disubstituted α-AAs.

Using biocatalytic methods to prepare structurally complex AAs is an increasingly attractive approach. Currently, many pharmaceutical companies (e.g. Merck & Co., Inc) and start-up biotech companies (e.g. Aralez Bio) have adopted this strategy. However, only a handful of biocatalysts are available to synthesize α,α-disubstituted α-AAs.

### SUMMARY OF THE DISCLOSURE

The disclosure generally describes various compounds, systems, and methods for performing Mannich cyclization to synthesize α,α-disubstituted α-AAs. A Mannich cyclase can be provided in solution and contacted with a substrate to yield an α,α-disubstituted α-AA. The substrate can be a single component or a two-component substrate. The Mannich cyclase can be LolT, a PLP-dependent enzyme in a biosynthetic gene cluster for synthesizing loline alkaloids. The Mannich cyclase can be expressed or produced by heterologous expression within an organism.

In some aspects, the techniques described herein relate to a method for yielding an azacyclic α-amino acid, including: providing a Mannich cyclase in a solution; and contacting the Mannich cyclase with a substrate.

In some aspects, the techniques described herein relate to a method, wherein the Mannich cyclase is LolT.

In some aspects, the techniques described herein relate to a method, wherein the solution includes a culture of live organisms heterologously expressing the Mannich cyclase.

In some aspects, the techniques described herein relate to a method, wherein the solution includes enriched Mannich cyclase that purified from organisms that heterologously expressed the Mannich cyclase; or wherein the solution includes an organismal lysate of organisms that heterologously expressed the Mannich cyclase.

In some aspects, the techniques described herein relate to a method further including: expressing the Mannich cyclase in the organisms, wherein the expression of Mannich cyclase is heterologous; and lysing the organisms to generate a lysate.

In some aspects, the techniques described herein relate to a method further including: enriching the Mannich cyclase from the lysate.

In some aspects, the techniques described herein relate to a method, wherein the substrate is selected from Substrate A, Substrate B, or Substrate C.

In some aspects, the techniques described herein relate to a method, wherein the substrate is Substrate A; wherein n is 1, 2, or 3; wherein m is 1, 2, or 3.

In some aspects, the techniques described herein relate to a method, wherein when n is 1, m is 1, 2 or 3, when n is 2 or 3, m is 1 or 2, when m is 1 or 2, n is 1, 2, or 3, and when m is 3, n is 1.

In some aspects, the techniques described herein relate to a method, wherein either n or m is not 1.

In some aspects, the techniques described herein relate to a method, wherein the substrate is Substrate B; wherein n is 1, 2, or 3; wherein m is 1, 2, or 3.

In some aspects, the techniques described herein relate to a method, wherein when n is 1, m is 1, 2 or 3, when n is 2 or 3, m is 1 or 2, when m is 1 or 2, n is 1, 2, or 3, and when m is 3, n is 1.

In some aspects, the techniques described herein relate to a method further including: deprotecting the substrate.

In some aspects, the techniques described herein relate to a method, wherein the substrate is Substrate C; wherein n is 1 or 2.

In some aspects, the techniques described herein relate to a method, wherein when n is 1, R is an alkyl, an alkenyl, a phenyl, a phenethyl, a naphthalenyl, or a pyridinyl.

In some aspects, the techniques described herein relate to a method, wherein when n is 2, R is an alkyl or an alkenyl.

In some aspects, the techniques described herein relate to a method further including: enriching a product generated from the substrate via the Mannich cyclase.

In some aspects, the techniques described herein relate to an organism, including: a nucleic acid including a sequence for heterologous expression a Mannich cyclase, wherein the Mannich cyclase is LolT.

In some aspects, the techniques described herein relate to an organism, wherein the organism is E. coli.

In some aspects, the techniques described herein relate to an organism, wherein the nucleic acid includes SEQ ID NO: 1.

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIGS. 1A-1C provide compound structures of substrates and products to be utilized or generated by a Mannich cyclase.

FIG. 1D provides an example of a method for producing larger molecules based on α,α-disubstituted α-AAs products generated via Mannich cyclization.

FIGS. 2A-2B provide schematics of PLP-dependent Mannichase and loline biosynthetic pathway. FIG. 2A provides a schematic showing mechanistic similarity between canonical PLP-dependent aldolases and the hypothetic PLP-dependent Mannichase. FIG. 2B provides a schematic showing incomplete biosynthetic pathway of loline alkaloids of which the formation of pyrrolizidine heterocycle remains unclear and an intramolecular Mannich cyclization step is likely involved.

FIG. 3 provides a schematic of conserved loline alkaloid biosynthetic genes across different fungal species.

FIGS. 4A-4D provides schematics and data identifying LolT as a PLP-dependent Mannich cyclase. FIG. 4A provides a schematic depicting an elucidated pathway for the pyrrolizidine formation of loline. FIG. 4B provides HPLC analysis showing the reactions catalysed by LolT and LolD. All reactions were repeated for at least 3 times. Products were derivatized with Fmoc-Cl and analyzed by HPLC using a C18 column (Phenomenex Kinetex, 1.7 μm, 2.0×100 mm) with a linear elution gradient of 2-98% MeCN—H₂O solvent system supplemented with 0.1% formic acid as additive. FIG. 4C provides DFT-calculated Mannich cyclization transition state for anti TS-1. FIG. 4D provides DFT-calculated Mannich cyclization transition state for syn TS-2.

FIG. 5 provides SDS-PAGE analysis of purified proteins used in the study.

FIGS. 6A-6C provides UV-Vis spectra of as-isolated LolT and LolD. FIG. 6A: UV-Vis spectra of LolT under basic condition (0.2 M NaOH) and buffered assay condition (pH 7.5). FIG. 6B: UV-Vis spectra of LolD under basic condition (0.2 M NaOH) and buffered assay condition (pH 7.5). FIG. 6C: UV-Vis spectra of free PMP and PLP under basic condition. The above spectra indicate presence of PLP cofactors in both LolT and LolD.

FIGS. 7A-7B provides data of control experiments testing the function LolT and LolD using alternative substrates. FIG. 7A: Incubating LolD (10 μM) with 2 (1 mM) in phosphate buffer (pH 7.5) for 12 hrs at 28° C. FIG. 7B: Incubating LolT (1 μM) with decarboxylated 2 (1 mM) in phosphate buffer (pH 7.5) for 12 hrs at 28° C. All reactions were derivatized with Fmoc-Cl (50 mM) and analyzed by HPLC and LCMS. Decarboxylated 2 was prepared in situ by global deprotection of 12b, synthesis of 12b.

FIGS. 8A-8B provides DFT calculated free energy profile of PLP-dependent Mannich cyclization reaction and different cyclization TS. The protonation states of residues are determined by using Propka program. The numbers below the 8 TSs with varying ring sizes are relative free energy barrier with reference to their corresponding INT1 (condensation adduct). The unit is kcal/mol.

FIG. 9 provides a schematic of biocatalytic synthesis of 1-azabyclic α-amino acids by LolT. ^{a}All the enzymatic reactions gave only single stereoisomers as indicated by LC-MS and NMR analysis. ^{b}lsolated yields after two steps. First step is global deprotection of all Boc groups. Second step is the enzymatic reaction after removing TFA and organic solvent.

FIGS. 10A-10B provide data of steady-state kinetic study of LolT and LolD. FIG. 10A: Kinetic analysis of LolT-catalyzed cyclization reactions reveals that LolT favors 5-endo cyclization. The results from both Fmoc-Cl and OPA derivatization were shown for comparison.

FIG. 10B: Kinetic analysis of LolD-catalyzed nonoxidative decarboxylation. All reactions were performed in assay buffer: phosphate buffer (pH 7.5), 50 μM PLP, corresponding substrate at various concentrations. For 5-endo cyclization, LolT was added to a final concentration of 0.1 μM and the reaction was quenched after 1 min; while for 6-endo cyclization, LolT was added to a final concentration of 1 μM and the reaction was quenched after 1 min, and for 7-endo cyclization, LolT was added to a final concentration of 10 μM and the reaction was quenched after 5 min. For decarboxylation reaction, LolD was added to a final concentration of 1 μM and the reaction was quenched after 20 min. The derivatization method was described in the methods section. All assays were performed in triplicates at 28° C. and individual point was shown on the plot. Except for reaction forming 5, all data were fitted to the standard Michalis-Menton equation. Because reaction leading to 5 showed unsaturation up to the substrate concentration provided, the data were essentially fitted to the linear equation

$$\frac{{v}_{0}}{[ E ]} = \frac{{k}_{cat}}{{K}_{M}} [ S ] .$$

FIGS. 11A-11C provide structural characterization of LolT. FIG. 11A provides a dimeric structure of holo-LolT and close-up view of covalently bound PLP cofactor. Monomer A is colored in green while monomer B is colored in light pink. PLP is highlighted in orange stick model. A Polder omit map of PLP is shown in blue mesh (contoured at the 3.0s level). Hydrogen bond interactions are shown as black dashed lines. FIG. 11B provides the active site tunnel (shown as black mesh) of monomer A is calculated by Caver, whereas the active site cavity volume is determined by GetCleft from NRGsuite. Monomer B is shown in ribbon representation with the atomic displacement parameters (B-factor) coded by thickness and color gradient. Note that the highly flexible active site capping loop is partially disordered from Ser283 to Thr291, which are shown as dashed lines. The 3₁₀-helix h4 carrying Trp279 is labeled. FIG. 11C provides a close-up view of the transition state docked in the active site, and the averaged conformation from the molecular dynamic simulations.

FIGS. 12A-12D provide molecular dynamics (MD) simulations of LolT. FIG. 12A: Conformational ensembles of LolT in complexation with TS-1 in 1 μs MD simulations. FIG. 12B: Calculated average root mean square fluctuation (RMSF) for each residue. FIG. 12C: RMSD curves of four replicas of holo-LolT. FIG. 12D: RMSD curves of four replicas of LolT-TS1. The conformation reported is the largest cluster resulted from cpptraj clustering analysis derived from combination of all four replicas. The unit of RMSF and RMSD is A, while the unit of MD step is nanosecond.

FIGS. 13A-13C provide schematics of fitting of the alternative transition states that explains stereoselectivity. FIG. 13A: TS is perfectly fit in the active site. FIG. 13B: TS-epi (iminium re-facial attack) is sterically blocked by Tyr125. FIG. 13C: The enantiomers of TS-epi (left) and TS (right) were also docked to the active site by overlaying with TS. The iminium side chain is not perfectly fit due to less space offered on the si-face of PLP. Note that flexible docking has been carried out on the diastereomeric TS, but it failed to generate any reasonable docking pose serving as a starting structure for the MD simulation. We thus fitted the antipodal TS into the active site manually, and using the resulting steric clash as the rationale on why one diastereomeric TS is preferred over the other.

FIG. 14 provides a schematic of fitting of the 6-endo and 7-endo transition states that explains observed trend in catalytic efficiency. Comparison with the docked 5-endo TS indicates the enzyme active site is not large enough to perfectly fit a 7-endo transition sate, which explains the dramatically decreased catalytic efficiency.

FIGS. 15A-15B provide schematics and data of a mutagenesis study and and proposed catalytic mechanism of LolT. FIG. 15A provides data of LolT mutant activity using 2 as substrates. The bar graph provides the mean±s.d. (n=3 independent experiments). Error bars represent standard deviation (s.d.) with a measure of center representing the mean. Open circles represent individual data points. Assay conditions: all activity was conducted with 1 mM substrate in phosphate buffer (pH 7.5) without free PLP cofactor included. For WT, the final enzyme concentration is 0.05 μM. For the mutant, the final enzyme concentration is from 1-10 μM. The reaction is quenched after 5 min and derivatized with excess amount of Fmoc-Cl prior to LC-DAD-MS analysis. FIG. 15B provides a proposed catalytic mechanism of LolT. Dashed straight lines indicate hydrogen bond or π-π stacking interactions. The conformation of iminium side chain is favored by cation-r interaction with Trp279(B) and its orientation is restricted by Y125.

FIGS. 16A-16D provides reaction schematics and data of hydrogen-deuterium exchange assay of LolT with diamino acid substrate analogues. All reactions were monitored by 1H-NMR spectra and boiled enzymes were used as control. For mass data analysis, the reaction mixtures were derivatized with Fmoc-Cl.

FIG. 17 provides chiral analysis of deuterated L-diamino acids. Deuterated amino acids were derivatized with Marfey's reagent and analyzed by HPLC on a C18 analytical column. Amino acids standards were derivatized and analyzed using the same method to serve as references. Note that regio-isomers were observed for L- and D-Dap, but the retention clearly indicates no D-Dap was produced during the hydrogen-deuterium exchange process.

FIGS. 18A-18B provide schematics of LolT-catalysed two-component Mannich cyclization reactions. FIG. 18A provides a concept of trapping LolT-generated carbanions with in situ formed imines. FIG. 18B provides data of isolation and characterization of selected quaternary α-amino acid products. The X-ray crystal structure of Boc-protected 14 confirmed the absolute configuration. The diastereoisomeric ratio and determined by HPLC after derivatization and NMR after isolation. The enantiomeric excess is determined by HPLC after derivatization with chiral reagents after isolation. ^{a}Reaction condition: KPi buffer (pH 7.5) with 10 μM LolT, 10 mM L-Dab, and 50 mM aldehyde at 18° C. for 2 h. ^{b}Reaction condition: KPi buffer (pH 7.5) with 10 μM LolT, 0.5 mM PLP, 10 mM L-Dab, and 50 mM aldehyde at 37° C. for 24 h. ^{c}Reaction condition: KPi buffer (pH 7.5) with cell lysate, 10 mM L-Dab, and 50 mM aldehyde at 28° C. for 16 h. ^{d}Reaction condition: KPi buffer (pH 7.5) with 20 μM LolT, 0.5 mM PLP, 10 mM L-Dab, and 50 mM aldehyde at 28° C. for 16 h.

FIGS. 19A-19B provide a table of structures and reaction conditions to determine substrate scope of LolT-catalyzed two-component Mannich cyclization reactions.

FIG. 20 provides steady-state kinetic analysis of two-component Mannich cyclization reaction with benzaldehyde and L-Dab as substrates. The intersection on this double-reciprocal plot indicates a ternary complex is formed in this balustrade reaction, which supports the formation of a PLP-bound imine intermediate.

FIGS. 21A-21B provide reaction schematics and data detailing reaction conditions that affect LolT-catalyzed two component Mannich cyclization reactions. FIG. 21A: The diastereoselective ratio between 11 and 12 is pH-dependent. Data represent the mean±s.d. (n=3 independent experiments). Error bars represent standard deviation (s.d.) with a measure of centre representing the mean. FIG. 21B: Extension of reaction time and elevation of reaction temperature favor the thermodynamic product, syn-isomer (12).

FIG. 22 provides DFT-calculated energy difference between 11 and 12. The numbers in the parentheses are relative free energy with reference to INT1 (condensation adduct) with kcal/mol as the unit. The energy difference between 11 and 12 is small even considering different possible charge states under reaction conditions.

FIGS. 23A-23D provide schematics of PLP-catalyzed nonenzymatic epimerization of 11 to 12. FIG. 23A: In the presence of free-PLP, 11 is epimerized to 12 and reaches equilibrium at room temperature. FIG. 23B: Structure-activity relationship of PLP-catalyzed epimerization. The phenoxide group is essential for the epimerization activity. FIG. 23C: Free benzaldehyde was not incorporated into 12 during this epimerization process, suggesting there is no breakdown of imine, even though retro-Mannich cleavage took place. FIG. 23D: proposed mechanism for PLP-catalyzed epimerization. According to this mechanistic proposal, electron-rich aldehyde would favor this process, such as another epimer product 21 isolated in this work.

FIG. 24 provides data showing LolD catalyzes non-oxidative decarboxylation. Assay condition: 10 μM LolD was incubated with 1 mM amino acid substrate (e.g. 3) in the presence of common α-keto acids (10 μM). The reaction is quenched with Fmoc-Cl derivatization reagent and analyzed on HPLC and LC-MS. In contrast to dialkylglycine decarboxylase, no tandem decarboxylation-transamination activity was observed with LolD. Transaminase assay with common proteinogenic amino acids did not show observable activity for LolD.

FIG. 25 provides schematics of representative examples of bioactive alkaloids harboring 1-azabicycloc[m.n.0]alkane core scaffolds.

FIG. 26 provides sequences of codon-optimized DNA sequences of LolT (SEQ ID NO: 1) and LolD (SEQ ID NO: 2) for E. coli expression.

### DETAILED DESCRIPTION

Turning now to the drawings and data, the numerous embodiments of the disclosure are directed towards compositions, systems and methods for performing Mannich cyclization reaction and applications thereof. In several embodiments, enzymatic catalysis is utilized to perform Mannich cyclization. Various enzymes have been discovered capable of performing Mannich cyclization, which can be referred to as Mannich cyclases. Accordingly, various embodiments are directed towards use of these enzymes. In some embodiments, one or more enzymes for catalyzing Mannich cyclization are purified or otherwise enriched, and utilized in solution to generate cyclic products. In some embodiments, one or more enzymes for catalyzing Mannich cyclization are heterologously expressed from a nucleic acid encoding said enzymes utilizing a biological expression system. In some embodiments, a transgenic organism comprises a nucleic acid encoding one or more enzymes for catalyzing Mannich cyclization, which can be utilized to produce said enzymes for catalysis. In some embodiments, a transgenic organism comprises a plurality of enzymes of a biochemical pathway, inclusive of an enzyme for catalyzing Mannich cyclization, to generate a product compound. Various embodiments are also directed to systems and methods to produce a product compound, including a therapeutic, in which the product compound comprises the use of a precursor compound generated via a Mannich cyclase.

In several embodiments, a Mannich cyclase utilizes the organic cofactor pyridoxal 5′-phosphate (PLP) to yield a product. PLP-dependent enzymes constitute a ubiquitous family of proteins and play a central role in metabolism and numerous cellular processes, catalyzing a diverse range of chemical reactions, including but not limited to, transamination, decarboxylation, racemization, epimerization, β/γ-elimination and substitution, aldol addition, Claisen condensation, and O₂-dependent oxidation reactions. Despite the prevalence of PLP-dependent enzymes in nature, prior to the work performed and described herein, no PLP-dependent enzymes were known to catalyze Mannich-type reactions.

PLP-dependent enzymes are capable of chemo-, site-, and stereo-selectivity that are difficult to achieve using chemical methods. Therefore, PLP-dependent enzymes are attractive biocatalysts, and many of them have been successfully employed to perform challenging chemical transformations on amine-containing substrates, including (but not limited to) biocatalytic asymmetric synthesis of noncanonical amino acid building blocks and chiral amine pharmaceuticals. And as described herein and in accordance with several embodiments, an enzyme referred to as LolT is a Mannich cyclase that is utilized to catalyze a Mannich reaction to yield an aza(bi)cyclic α-amino acid. LolT is a PLP-dependent enzyme that is expressed from fungal biosynthetic gene clusters that generate loline alkaloids.

In accordance with various embodiments, substrates that can be utilized to generate products comprise cyclic iminiums comprising a ring size from 5- to 7-membered. In some embodiments, a single component substrate is utilized, which can be a linear alkyl substrate is utilized as a substrate in a two-step reaction, in which the first step forms an intermediate cyclic iminium and then converted to an aza(bi)cyclic α-amino acid product via a Mannich cyclase (e.g., LolT). In some embodiments, cyclization via a Mannich cyclase (e.g. LolT) yields a stereoselective product. In some embodiments, cyclization via a Mannich cyclase (e.g. LolT) yields an α,α-disubstituted α-amino acid. In some embodiments, cyclization via a Mannich cyclase (e.g. LolT) yields a 5-endo, 6-endo, or 7-endo aza(bi)cyclic α-amino acid product.

In some embodiments, a two-component substrate is utilized, in which a first component is a diamino acid and a second component is an aldehyde to yield an azacyclic product via the Mannich cyclase (e.g., LolT). In some embodiments, to yield a pyrrolidine α-amino acid, the diamino acid utilized is 2,4-diaminobutyric acid. In some embodiments, to yield a piperidine α-amino acid, the diamino acid utilized is ornithine.

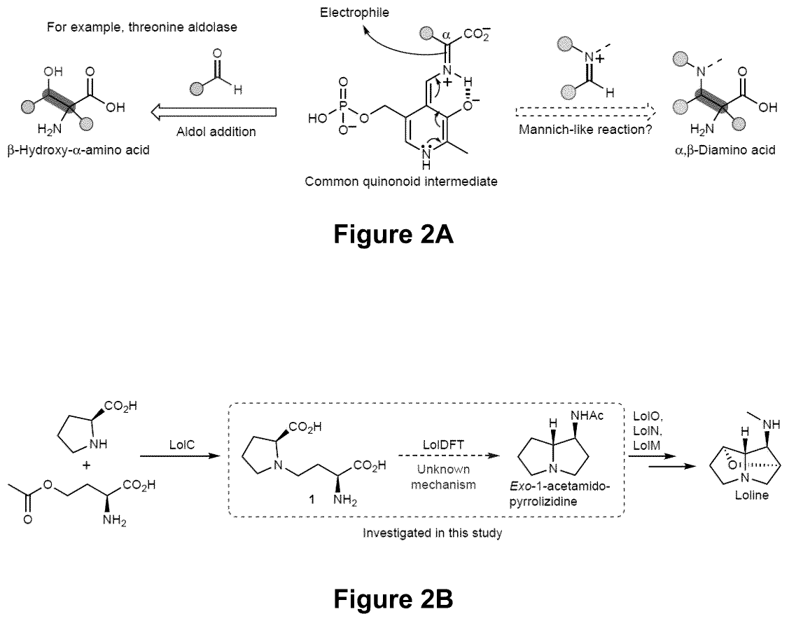

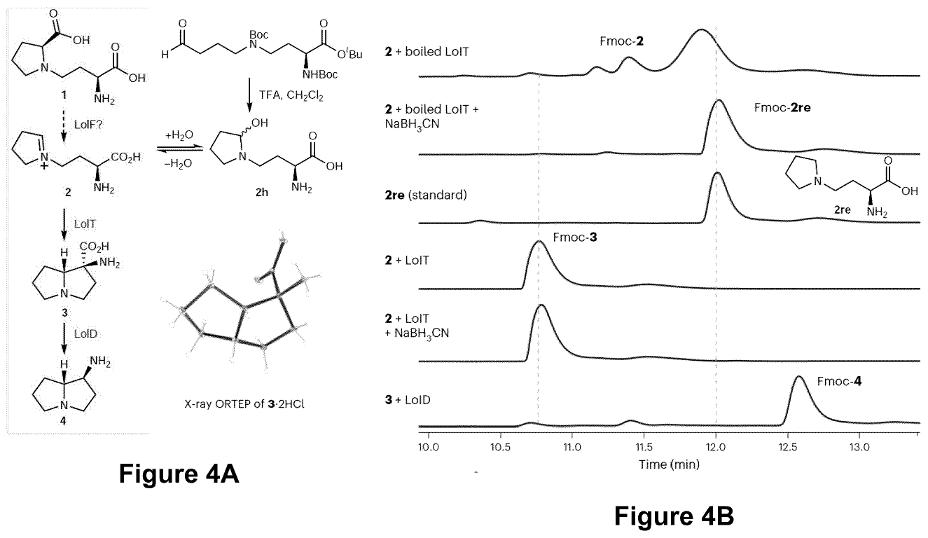

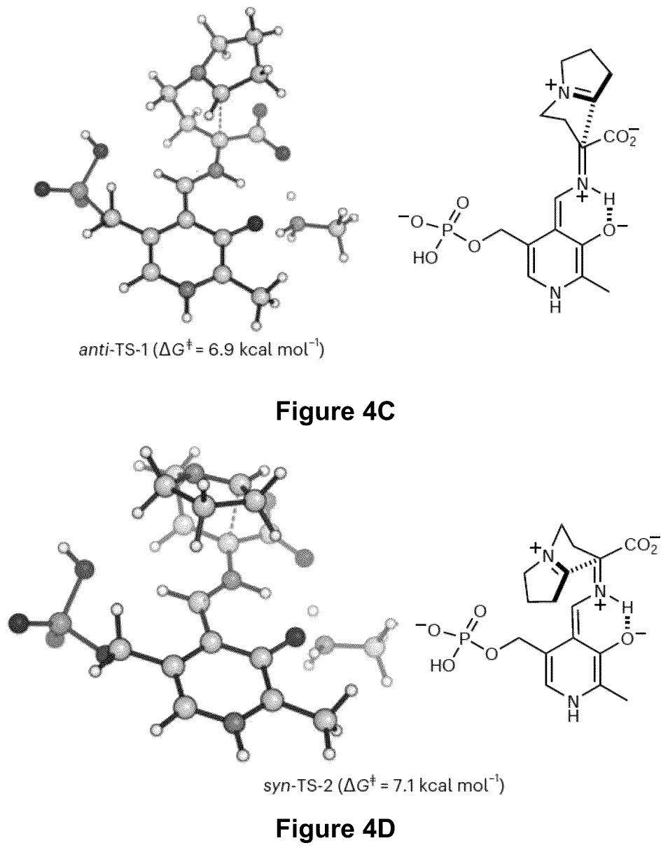

Loline alkaloids, a family of saturated pyrrolizidine natural products mainly produced by endophytic fungi, have been known for decades owing to their unusual highly strained tricyclic structure and intriguing insecticidal activities. The biosynthetic gene cluster for loline is reported and verified, and the biosynthetic pathway has been partially mapped out, including formation of the characteristic ether bridge (FIG. 2B). However, the biosynthetic mechanism of the pyrrolizidine heterocycle remained unclear. Previous isotope labeling and precursor feeding experiment confirmed the intermediacy of the amino acid precursor N-(3-amino-3-carboxypropyl)proline (1), suggesting an oxidative cyclization route to construct the pyrrolizidine core scaffold from 1 (FIG. 2B). Embodiments herein utilize a mechanism involving LolT and LolD enzymes to transform proposed intermediate 2 to the pyrrolizidine, of which an enzymatic Mannich reaction must operate (FIG. 4A).

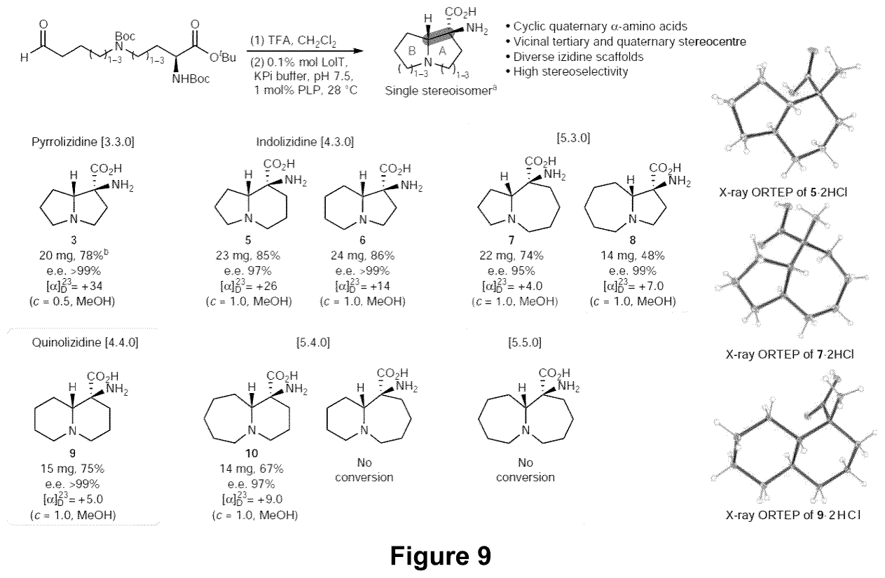

To show enzymatic activity, and in accordance with various embodiments, LolT resulted in a complete conversion of 2 to 3 without any intermediate product remaining, and no such reaction or conversion was observed with LolF or LolD. Additionally, no spontaneous conversion of 2 to 3 occurs. When LolD was added to a solution containing isolated 3, decarboxylation was observed. Isolation and structure characterization of the decarboxylated product 4 revealed it to be the (+)-exo-1-amino-pyrolizidine. These results indicate LolT catalyzes a 5-endo-trig Mannich annulation reaction whereas LolD is an α-quaternary amino acid decarboxylase. Additionally, LolT is capable of catalyzing 6- and 7-endo-trig reactions (in addition to 5-endo-trig mentioned previously) using similar iminium substrates, as illustrated in FIG. 9. Although the catalytic efficiency is decreasing with increased ring size, near complete conversion can still achieve with excellent diastereo- and enantiostereo-selectivity. Additionally, LolT can tolerate substrate analogues varying the B ring and resultant A ring sizes, and can catalyze the stereoselective Mannich-annulation reactions to afford a series of enantiomeric azabicyclic α-quaternary amino acids, such as pyrrolizidine and indolizidine heterocycles.

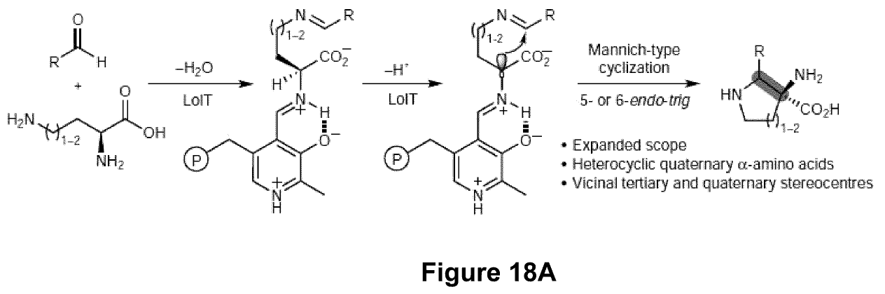

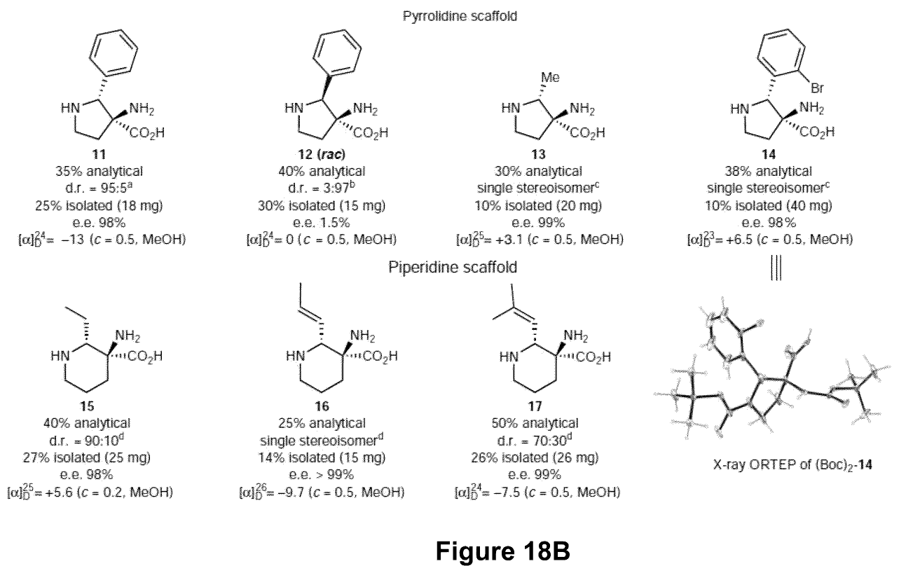

In some embodiments, two components are utilized as a substrate, where an aldehyde can form an imine adduct with the terminal amino group from a diamino acid. Upon deprotonation by LolT, such imine intermediate can be intercepted by the nucleophilic Ca carbanion, resulting in a net-cyclization reaction. LolT can catalyze both 5-endo and 6-endo intramolecular Mannich cyclization reaction in this bi-substrate system, as illustrated in FIG. 18A.

Several embodiments are directed towards generating α-amino acids. It has been discovered that the PLP-dependent enzyme LolT is a Mannich cyclase capable of yielding an azacyclic α-amino acid product. Accordingly, a reaction solution can be prepared comprising a substrate and a Mannich cyclase (e.g., LolT) to generate a variety of azacyclic α-amino acid products. The various azacyclic α-amino acid products can purified or otherwise enriched from the reaction solution. Various means can be utilized to purify or enrich a product, such as ion exchange, chromatography, distillation, filtration, crystallization, centrifugation, differential extraction, etc. The various means for purification and/or enrichment can be combined and/or utilized sequentially, as appropriate.

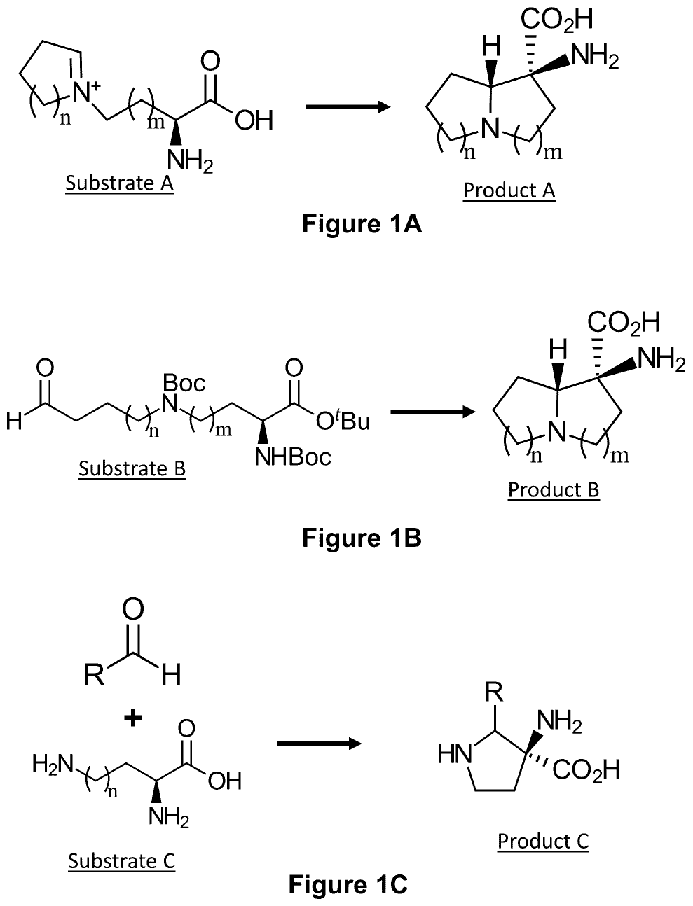

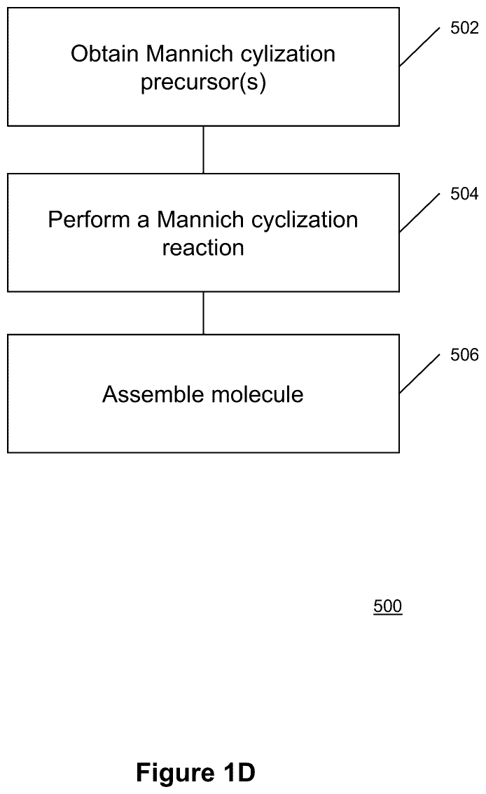

Provided in FIG. 1A is a reaction schematic in which Substrate A is converted into Product A via contact with a Mannich cyclase. In several embodiments, the Mannich cyclase is LolT. In some embodiments, n and m are each independently 1, 2, or 3. In some embodiments in which n is 1, m is 1, 2, or 3. In some embodiments in which n is 2 or 3, m is 1 or 2. In some embodiments in which m is 1 or 2, n is 1, 2, or 3. In some embodiments in which m is 3, n is 1. In some embodiments, at least one of n or m is not 1. See step 2 of FIG. 9 for examples of reaction conditions for various substrates and products. In some embodiments, Product A is contacted with a decarboxylase to remove the carboxylic acid. In some embodiments, the decarboxylase in LolD.

Provided in FIG. 1B is a reaction schematic in which Substrate B is converted into Product B via a 2-step reaction and contact with a Mannich cyclase. In several embodiments, the Mannich cyclase is LolT. In some embodiments, Substrate B is first deprotected via TFA in an organic solvent, and then is subsequently contacted with the Mannich cyclase. In some embodiments, n and m are each independently 1, 2, or 3. In some embodiments in which n is 1, m is 1, 2, or 3. In some embodiments in which n is 2 or 3, m is 1 or 2. In some embodiments in which m is 1 or 2, n is 1, 2, or 3. In some embodiments in which m is 3, n is 1. In some embodiments, at least one of n or m is not 1. See FIG. 9 for examples of reaction conditions for various substrates and products. In some embodiments, Product B is contacted with a decarboxylase to remove the carboxylic acid. In some embodiments, the decarboxylase in LolD. It should be understood that Substrate B can be provided utilizing a variety of protecting groups and any means for deprotection appropriate to the protecting groups can be utilized.

Provided in FIG. 1C is a reaction schematic in which Substrate C is converted into Product C via contact with a Mannich cyclase. In several embodiments, the Mannich cyclase is LolT. In some embodiments, Product C is contacted with a decarboxylase to remove the carboxylic acid. In some embodiments, the decarboxylase in LolD.

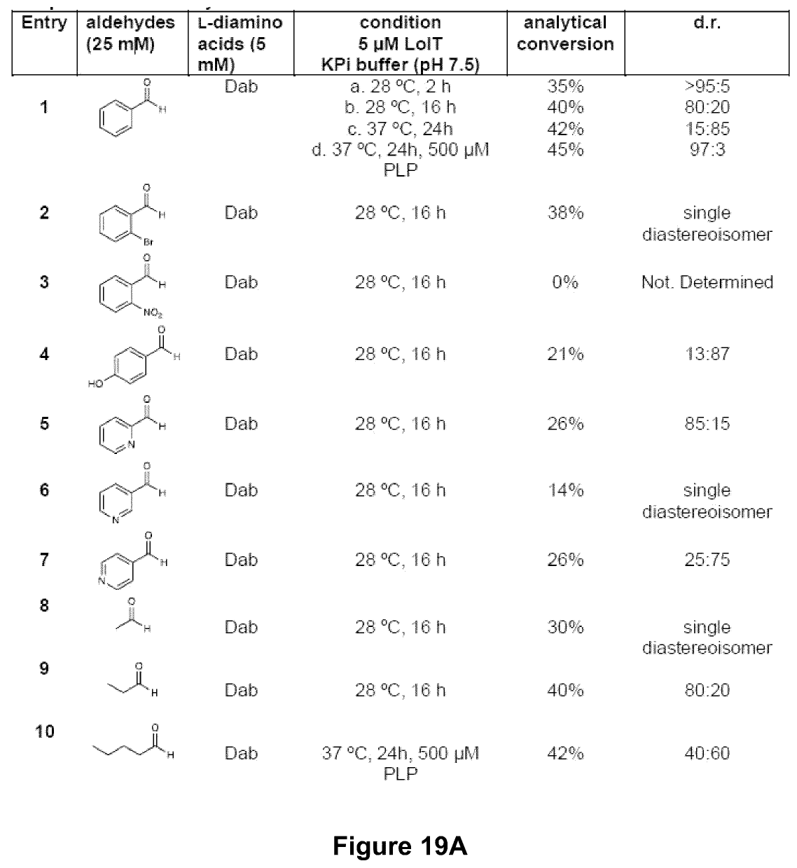

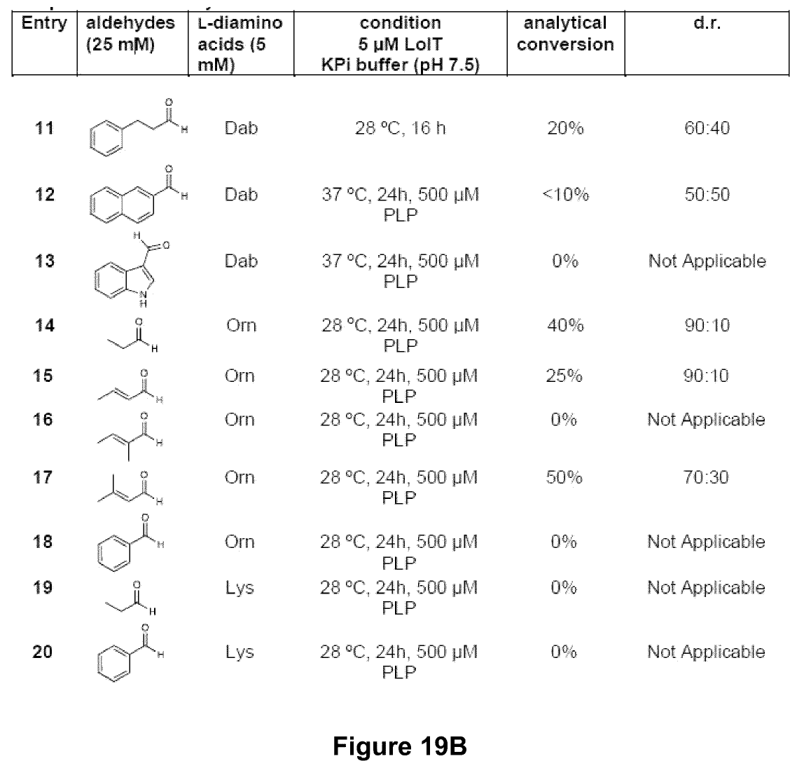

As depicted in FIG. 1C, Substrate C is a two-component substrate inclusive of a diamino acid and an aldehyde. In some embodiments, the diamino acid is 2,4-diaminobutyric acid (Dab). In some embodiments, the diamino acid is ornithine (Orn). Various aldehydes can be utilized, which may be dependent on the diamino acid. In some embodiments in which Dab is utilized as the diamino acid, the R group of the aldehyde is selected from an alkyl, an alkenyl, a phenyl, a phenethyl, a naphthalenyl, or a pyridinyl. In some embodiments in which Dab is utilized as the diamino acid, the R group of the aldehyde is an alkyl selected from a methyl, an ethyl, a propyl, or a butyl. In some embodiments in which Dab is utilized as the diamino acid, the R group of the aldehyde is an alkenyl selected from a vinyl, a propenyl, or a 2-methylpropenyl. In some embodiments in which Dab is utilized as the diamino acid, the R group of the aldehyde is a phenyl substituted with a halide or a hydroxide; in some embodiments the halide is chloride, and in some embodiments the halide is bromide. In some embodiments in which Orn is utilized as the diamino acid, the R group of the aldehyde is selected from an alkyl or an alkenyl, In some embodiments in which Orn is utilized as the diamino acid, the R group of the aldehyde is an alkyl selected from a methyl, an ethyl, a propyl, or a butyl. In some embodiments in which Orn is utilized as the diamino acid, the R group of the aldehyde is an alkenyl selected from a vinyl, a propenyl, or a 2-methylpropenyl. See FIGS. 18B-19B for examples of reaction conditions for various substrates and products.

Due to the ability of LolT to catalyze such reactions, additional embodiments utilize LolT to create and assemble larger molecules, including therapeutics. In such embodiments, LolT is used to generate an α-AA which can be used as a precursor for the larger molecules. Such embodiments have higher reaction efficiency and/or fewer byproducts (or off-target products). Such embodiments thus reduce waste and cost in production and also require less input. Turning to FIG. 1D, an exemplary method 500 to produce a larger molecule is illustrated. At 502, certain embodiments obtain one or more substrates for a Mannich cyclization reaction. At 504, certain embodiments perform a Mannich cyclization reaction. Such embodiments can include one or more enzymes, such as described herein. The product of the reaction can be assembled into a larger molecule at 506. Examples of substrates, reactions, and products that can be utilized are provided within FIGS. 1A-1C. Certain embodiments include additional precursors (such as precursors obtained by additional Mannich cyclization reactions) or obtained from an outside source, such as through synthetization reactions (biochemical or chemical) or obtained commercially.

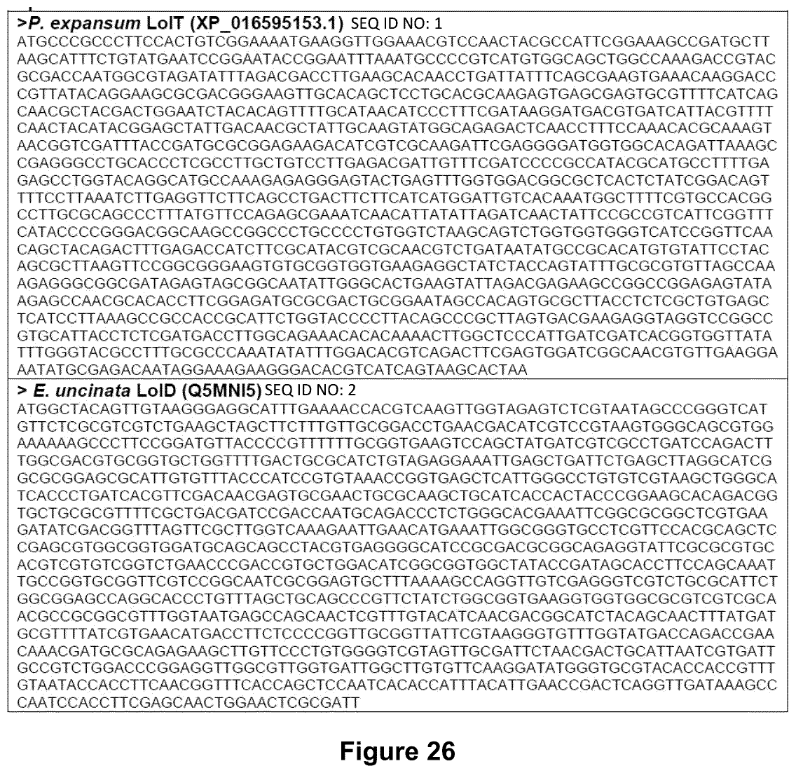

Several embodiments are directed toward expression systems for expressing and/or producing a Mannich cyclase (e.g., LolT). In many embodiments, an organism is configured to provide heterologous expression the Mannich cyclase(e.g., LolT), such that the transgene to be expressed is derived from a different species than the host organism. Various organism can be utilized, such as (for example) yeast, bacteria, or fungi. In one example, the organism is E. coli, but it should be understood any useful organism for heterologous expression can be utilized. In many embodiments, the host organism comprises a nucleic acid comprising a sequence for expressing a Mannich cyclase (e.g., LolT), which can be codon optimized for expression in that organism. An example of a codon optimized sequence for expression of LolT in E. coli is provided in FIG. 26 (see SEQ ID NO: 1).

One manner in which a host organism can express a Mannich cyclase (e.g., LolT) is to express the polypeptide gene product from a polynucleotide construct, which can be DNA-based, RNA-based, a DNA/RNA-hybrid, or a polynucleotide derivative thereof (e.g., LNA).

A DNA-based polynucleotide construct can be circular (e.g., plasmid) or linear, and can be integrated into the host genome or exist as an episome. A DNA-based polynucleotide construct can comprise the sequence for encoding the polypeptide gene product and other sequences for providing and/or regulating expression. Such sequences can include a promoter, an enhancer, a Shine-Dalgarno sequence, an intron, a polyadenylation signal, or a regulatory operon each of which can individually be operably linked to the sequence for encoding the polypeptide gene product. Expression of the a Mannich cyclase (e.g., LolT) can be constitutive or conditional, based on the operably linked sequences for providing and/or regulating expression. A DNA-based polynucleotide can further comprise cloning sequences (e.g., restriction enzyme sites), and/or sequences for DNA replication. Methods of cloning and propagation are described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)).

An RNA-based polynucleotide construct can be circular or linear. An RNA-based polynucleotide construct can comprise the sequence for encoding the polypeptide gene product, other sequences for providing translation, and optionally sequences for providing RNA stability. Such sequences can include a ribosomal binding site, a 5′ untranslated region, a 3′ untranslated region, or a poly-A tail, each of which can individually be operably linked to the sequence for encoding the Mannich cyclase (e.g., LolT).

Various embodiments can include additional enzymes to create a genetic or biochemical pathway to produce a product. For example, various genes involved loline alkaloid synthesis can be utilized, such that an earlier precursor of the can be utilized as a substrate or a downstream compound is synthesized in which the product of LolT is an intermediate compound. The additional enzymes can be expressed and produced in the same host organism, or alternatively, each additional enzyme can be expressed and produced in a different host organism and combined later. In one example, lolDFT is utilized to express LolD, LolF, and LolT in a host.

In many embodiments, Mannich cyclization is performed utilizing a Mannich cyclase (e.g., LolT) expressed and/or produced in a host organism. There are various means in which the expressed Mannich cyclase (e.g., LolT) can be utilized. In some embodiments, the reaction is performed in live host organism, in which the substrate is added to live culture of organisms expressing a Mannich cyclase (e.g., LolT) to yield the product. In some embodiments, a lysate of host organism is generated that comprises a Mannich cyclase (e.g., LolT), and the reaction is performed in lysate by adding the substrate to the lysate to yield the product. And in some embodiments, a Mannich cyclase (e.g., LolT) is extracted or otherwise enriched from a host organism culture or lysate, and the reaction is performed in a solution of the Mannich cyclase and the substrate. Various factors other factors can be added to the reaction to enhance the reaction. One factor that could be useful to add to a reaction mixtures when utilizing LolT is PLP. Concentrations of Mannich cyclase, substrate, and various factors, and other reaction conditions can be tuned or optimized to the requirements of the reaction and product to be generated.

In some embodiments, a Mannich cyclase (e.g., LolT) can be modified and/or optimized for various purposes, such as altering or improving throughput, efficiency, ability to catalyze additional compounds or precursors, and/or have additional biochemical properties, such as (but not limited to) stability, solubility, etc. In some embodiments, modifications to a Mannich cyclase increase stability at higher temperatures, which, for example, can be achieved by increasing the ability to form disulfide bridges within the enzyme. And in some embodiments, modifications to a Mannich cyclase increase solubility such that the enzyme can have greater yields in aqueous environments and/or organic environments, which may be useful if one or more compounds (e.g., product) has better solubility in one type of solution.

### EXAMPLES

The various embodiments of the disclosure are supported by experimental data results, which describe the discovery and applications of various compositions, systems, and methods for performing Mannich cyclization. Within these examples, pyridoxal 5′-phosphate (PLP)-dependent enzymes capable of catalyzing Mannich-type reactions are described. For example, LolT is a PLP-dependent enzyme that catalyzes a stereoselective intramolecular Mannich reaction to construct the pyrrolizidine core scaffold in Ioline alkaloids. In one example, the enzyme was exploited for stereoselective synthesis of α,α-disubstituted α-amino acids.

### A Pyridoxal 5′-Phosphate-Dependent Mannich Cyclase

Enzymes that utilize pyridoxal 5′-phosphate (PLP), arguably the most versatile organic cofactor, constitute a ubiquitous family of proteins and play a central role in metabolism and many cellular processes. These PLP-dependent enzymes catalyze a diverse range of chemical transformations, including but not limited to, transamination, decarboxylation, racemization, epimerization, [3+2] annulation, b/g-elimination or replacement, aldol addition, Claisen condensation, and O₂-dependent oxidation reactions. Except for glycogen phosphorylase, the catalytic versatility of PLP-dependent enzymes mainly arises from the ability of PLP to function as an electron-sink, thereby stabilizing different types of reaction intermediates for subsequent transformations. Moreover, these PLP-dependent enzymes also show remarkable chemo-, site- and stereo-selectivity that are otherwise challenging to achieve using chemical methods. Therefore, PLP-dependent enzymes are extraordinary biocatalysts and play essential roles in natural product biosynthesis, as well as asymmetric synthesis of noncanonical amino acid building blocks and chiral amine pharmaceuticals.

Despite the vast number of PLP-dependent enzymes characterized to date, none of them is known to catalyze Mannich-type reactions, one of the most powerful synthetic methods in organic chemistry to build carbon-carbon (C—C) bonds. Based on our mechanistic understanding of PLP enzymology, we postulate that such enzymatic function (i.e. Mannichase activity) is conceptually viable due to the mechanistic similarities with many Ca-alkylating PLP-dependent enzymes, such as threonine aldolases and serine hydroxymethyltransferases (FIG. 2A). Indeed, recent development of bio-inspired pyridoxal-based organocatalysts for asymmetric Mannich reactions to make α,β-diamino acids provided the molecular basis for their enzymatic counterparts.

Motivated by the synthetic utility of enzymatic Mannich reactions for stereoselective C—C bond formation (e.g. carboxymethylproline synthase), and the unmet need to broaden the scope of C—C bond forming biocatalysts, we set out to identify PLP-dependent enzymes potentially catalysing Mannich-type reactions from natural product biosynthetic pathways, a rich source for enzyme discovery. In this example, we report the discovery of a PLP-dependent Mannich cyclase, LolT, which catalyzes the key biosynthetic formation of the pyrrolizidine core of Ioline alkaloids. In addition, we demonstrated its synthetic potential for stereoselective biocatalytic synthesis of a myriad of conformationally constrained aza(bi)cyclic quaternary α-amino acids bearing vicinal quaternary-tertiary stereocenters. We also determined the X-ray crystal structure of LolT to 2.1 Å and studied the structure-function relationship to understand its molecular mechanism.

### Results

### Discovery of a PLP-Dependent Mannich Cyclase

Loline alkaloids, a family of pyrrolizidine natural products produced mainly by endophytic fungi, are well-known for their highly strained tricyclic structure and insecticidal activities. Since the initial report of its biosynthetic gene cluster (BGC) almost two decades ago, the biosynthetic pathway to loline has been partially mapped out (M.J Spiering, et al., Genetics 169, 1403-14 (2005), the disclosure of which is hereby incorporated by reference). Notably, the oxygenase LolO responsible for the installation of ether bridge has been characterized in detail (J. Pan, et al., Biochemistry 57, 2074-2083 (2018); and J. Pan, et al., J. Am. Chem. Soc. 141, 15153-15165 (2019); the disclosures of which are hereby incorporated by reference). However, it remains a puzzle how the pyrrolizidine core scaffold is constructed from the committed precursor 1 (FIG. 2B). Although a minimal set of genes (lolDFT) were shown to be necessary and sufficient for the biosynthesis of intermediate exo-1-acetamido-pyrrolizidine in vivo, the exact enzymatic function of each gene product is unknown and no other biosynthetic intermediates has been observed besides 1 (FIG. 2B) (D. X. Zhang, et al., Fungal Genet Biol 46, 517-30 (2009); and D. J. F. Fleetwood, et al., PCT Pat. Pub. WO2019123399A1 (2019); the disclosures of which are incorporated herein by reference). Since retrobiosynthetic analysis suggests that a Mannich-type cyclization might be involved in the formation of the pyrrolizidine heterocycle, here we aimed to elucidate these hidden steps in loline biosynthesis (involving enzymes LolDFT) and to uncover the potential Mannich cyclization enzyme(s).

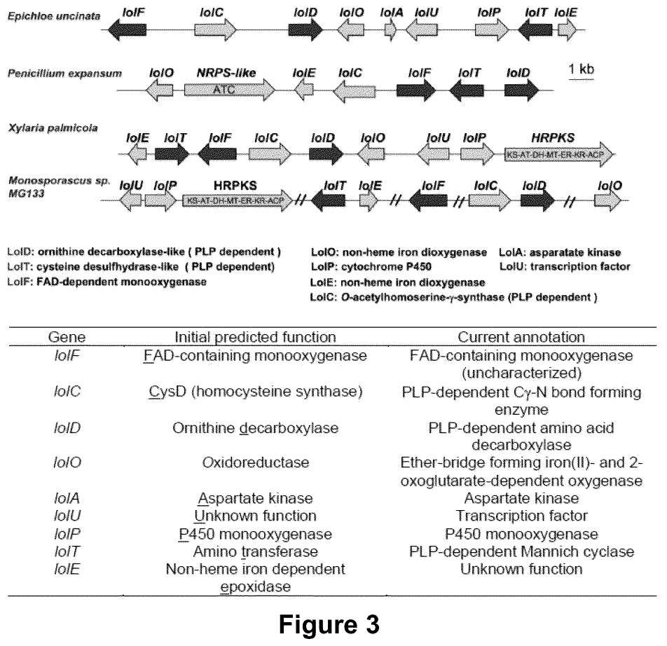

Close inspection of loline BGCs revealed that genes lolDFT are strictly conserved across different loline alkaloid producing species (FIG. 3). Based on their annotated gene function (lolD encodes a PLP-dependent amino acid decarboxylase, lolF encodes a flavin-dependent monooxygenase, while lolT encodes a PLP-dependent cysteine desulfhydrase-like protein) and the current understanding of flavin and PLP enzymology, we tentatively propose that the oxygenase LolF may catalyze an oxidative decarboxylation converting 1 to a hypothetical cyclic iminium intermediate (2); and the remaining two PLP-dependent enzymes (LolT and LolD) may further transform 2 into the desired pyrrolizidine heterocycle, although the timing of cyclization and decarboxylation is not clear (FIG. 4A). Finally, an endogenous N-acetyltransferase may install the acetyl group to account for the isolation of the exo-1-acetamido-pyrrolizidine intermediate (FIG. 2B).

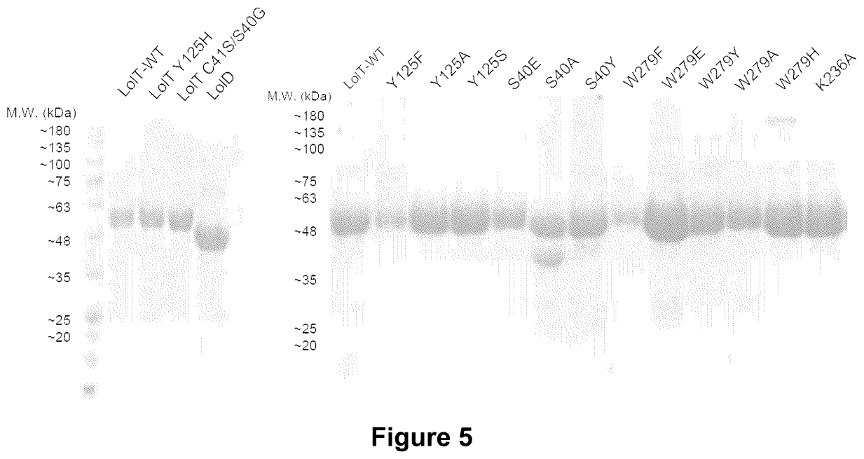

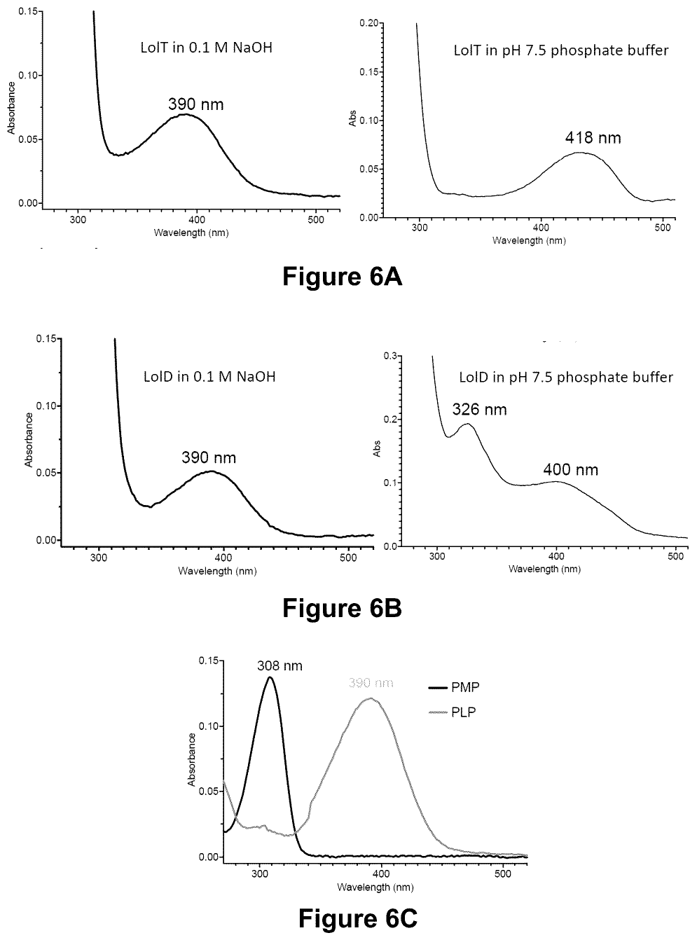

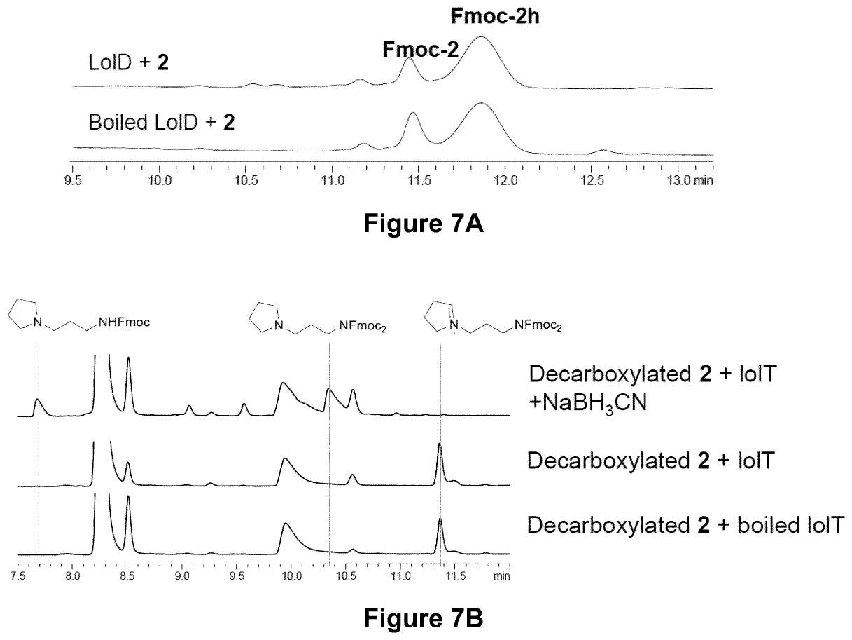

To test our hypothesis and unravel the biosynthetic mechanism of pyrrolizidine ring in loline, we took a biochemical approach by overexpressing the three candidate enzymes in E. coli and functionally characterizing them in vitro. The two PLP-dependent proteins were readily overexpressed and purified to homogeneity by Ni-affinity chromatography (FIG. 5). As-isolated LolT and LolD are yellow, and their absorbance spectra indicate the presence of the enzyme-bound PLP-cofactor (FIGS. 6A-6C). On the other hand, LolF was insoluble in spite of many attempts with different methods. Thus, we decided to bypass LolF and to chemically synthesize the proposed iminium intermediate. Compound 2 was afforded from global deprotection of its aldehyde precursor followed by spontaneous cyclization. According to the literature, under our assay condition, 2 should exist mainly in the iminium form (G. Houen, et al., Bioorg Med Chem 13, 3783-96 (2005); and I. Slabu, et al., ChemCatChem 8, 1038-1042 (2016); the disclosures of which are hereby incorporated by reference). Presence of the reactive cyclic iminium moiety was indirectly confirmed by in situ reduction with NaBH₃CN (FIG. 4B). Although incubating 2 with LolD showed no effect (FIGS. 7A-7B), addition of LolT converted the equilibrium mixture of 2 to a single product 3. No spontaneous conversion occurred in the absence of LolT. Product 3 shows the same molecular weight as 2 but is resistant to NaBH₃CN reduction (FIG. 4B). Large-scale reaction allowed us to isolate 3 and determine its structure as a pyrrolizidine α-quaternary amino acid with anti-configuration. Its absolute stereochemistry was ascertained by X-ray crystallography. To establish the biosynthetic relevance of 3, we further incubated 3 with LolD, and observed quantitative conversion of 3 to a decarboxylated product (4). Isolation and structure characterization of 4 revealed its identity as (+)-exo-1-amino-pyrrolizidine, suggesting that decarboxylation occurred with retention of configuration. The stereochemistry at the vicinal tertiary carbons (Cα and Cβ) of 4 is in accordance with natural loline alkaloids and previously characterized biosynthetic intermediate exo-1-acetamido-pyrrolizidine (FIGS. 2A-2B), which confirms its intermediacy in Ioline biosynthesis. To corroborate our conclusion that the pyrrolizidine scaffold of Ioline is constructed through Mannich cyclization followed by decarboxylation, we also synthesized the decarboxylated analogue of 2 and incubated it with LolT. As expected, no cyclization was observed (FIGS. 7A-7B). Taken together, our results unambiguously demonstrated that LolT is a PLP-dependent enzyme catalyzing Mannich-like reaction, while LolD is a PLP-dependent decarboxylase acting on quaternary α-amino acids.

LolT is a versatile Mannich cyclase

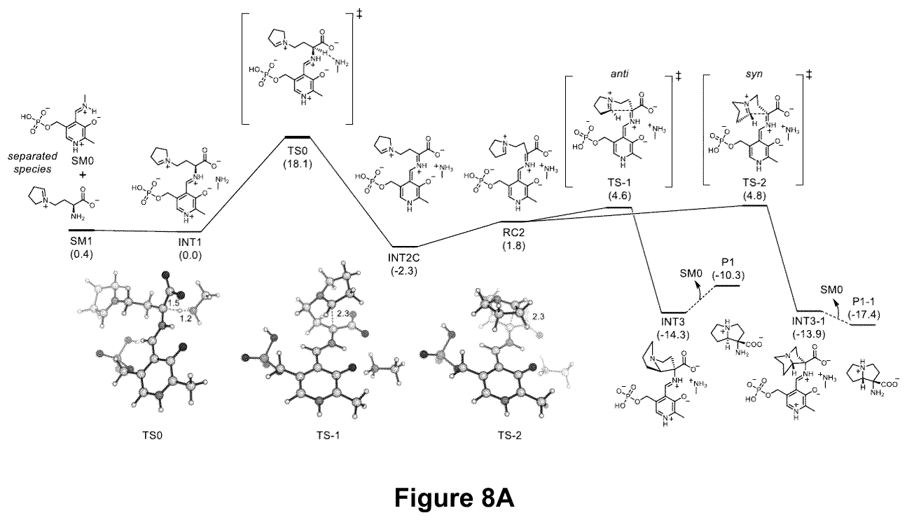

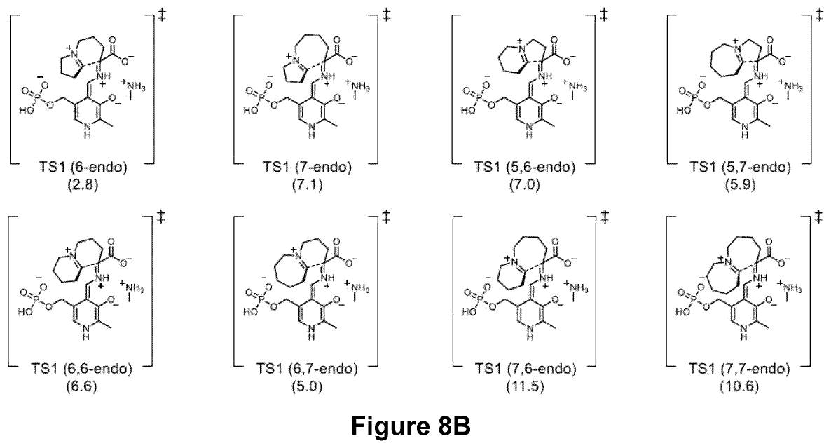

Since LolT is an unusual PLP-dependent enzyme shown to catalyze a Mannich-type cyclization reaction, we next performed DFT calculations to study the intrinsic reactivity and selectivity of this reaction (FIGS. 8A-8B). The isolated anti product 3 is found to be 7.1 kcal/mol thermodynamically less stable than the corresponding syn epimer product, which is not observed in our reactions. More importantly, the free energy barriers for the stereoisomeric transition states are about the same: 6.9 kcal/mol (TS-1) for anti cyclization (i.e. attack at the si-face of iminium) while 7.1 kcal/mol (TS-2) for syn cyclization (i.e. attack at the re-face of iminium) (FIGS. 4C-4D). These results suggest the observed Mannich-cyclization is stereo-controlled by LolT and 3 is a kinetic product.

With the unusual Mannich cyclase activity established for LolT, we next explored its synthetic potential for the stereoselective synthesis of various quaternary α-amino acids harboring diverse 1-azabicyclo[m.n.0]alkane (“izidine”) scaffolds. We tested a series of substrate analogues with systematic variations on the chain lengths. The substrate scope of LolT is shown to be broad (FIG. 9): not only can it tolerate different cyclic iminiums ranging in ring size from 5 to 7-membered (i.e. B ring), but can also catalyze 6- and 7-endo cyclization, although in the latter case B ring size is limited to 5-membered. For each reactive substrate, only one stereoisomeric product was observed. All products were isolated and fully characterized. The absolute configurations of 5, 7 and 9 were also determined by X-ray crystallography. Structural comparison of these products shows uniform stereochemical outcome at the two vicinal stereocenters (i.e. anti-configuration) and high enantiomeric excess, indicating that each cyclization is both enantio- and diastereo-controlled by LolT.

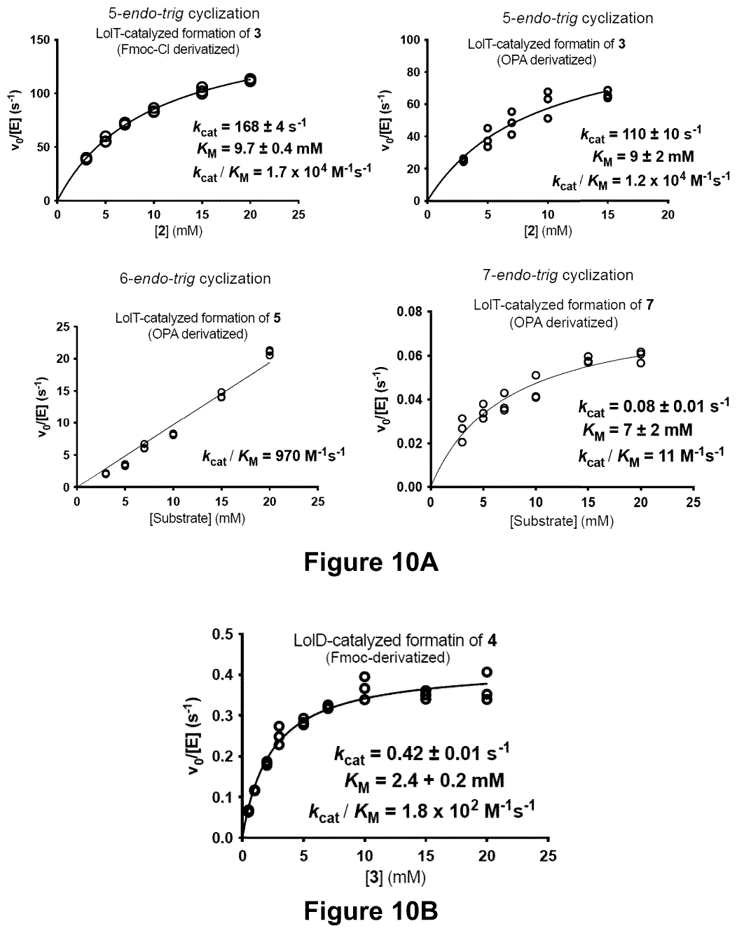

Steady-state kinetic analysis shows LolT efficiently catalyzes 5-endo-trig Mannich cyclization to make 3 with a k_{cat} of 110±10 s⁻¹ and K_{M} of 9±2 mM (FIGS. 10A-10B). In comparison, the catalytic efficiency decreases by ˜ 12-fold for 6-endo-trig cyclization (k_{cat}/K_{M} of 9.7×10² M⁻¹s⁻¹ to make product 5) and decreases by almost 1,000-fold for 7-endo-trig cyclization (k_{cat}/K_{M} of 11 M⁻¹s⁻¹ to make product 7). We reason that the compromised catalytic efficiency for expanded ring closures (i.e. A ring) are presumably due to steric effect: the enzyme active site pocket is not large enough to accommodate the expanded transition state conformation for 7-endo cyclization, in particular when B ring size also increases.

### Structural Insights for LolT-Catalyzed Mannich Cyclization

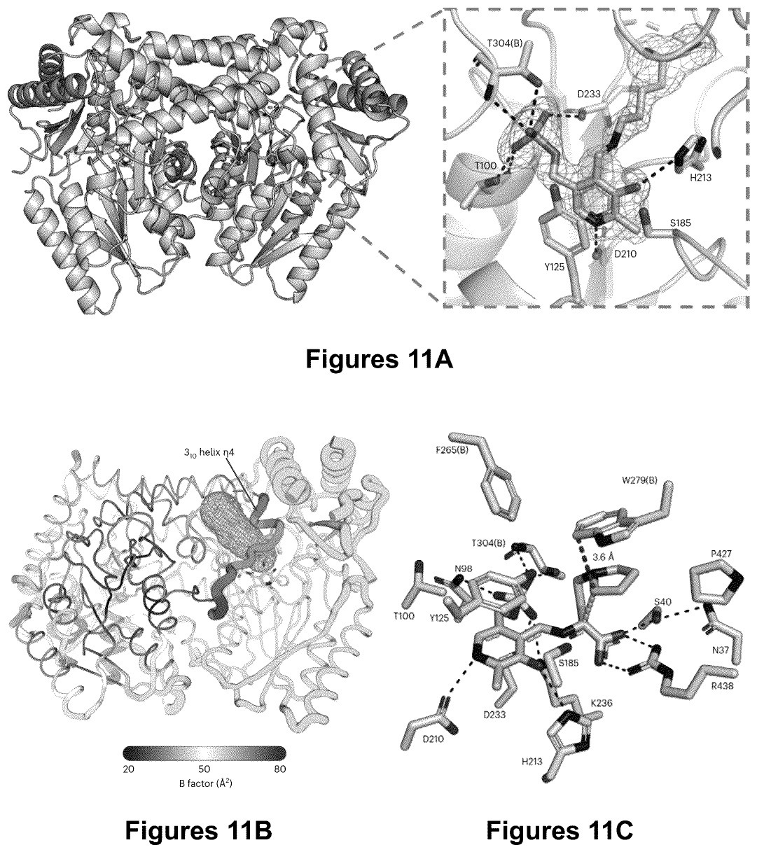

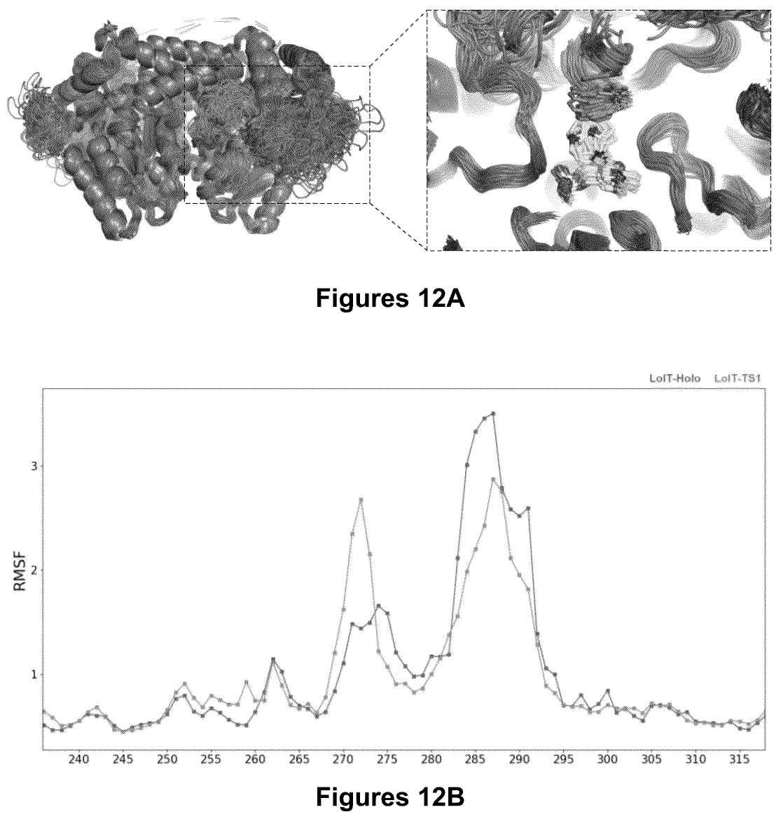

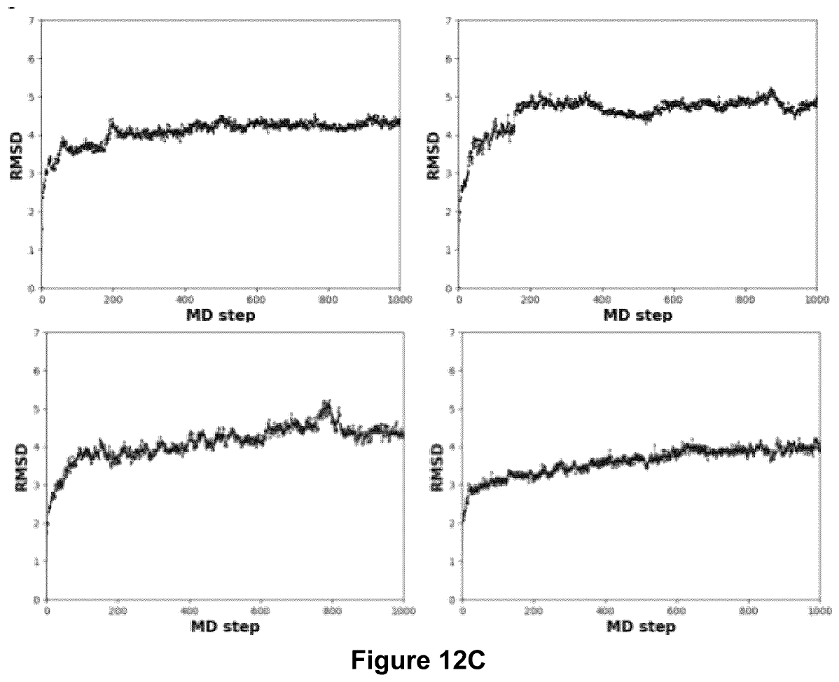

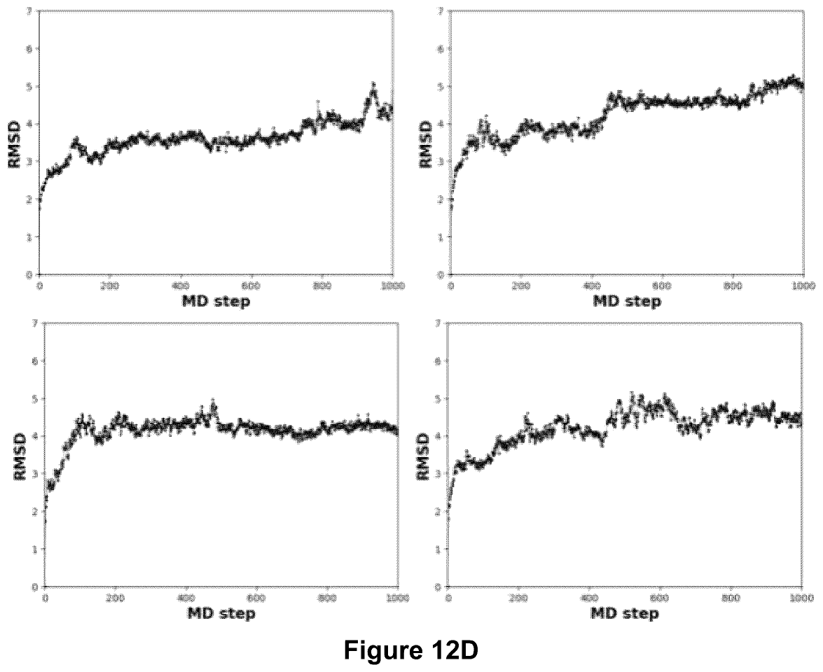

To understand LolT's function at molecular level, particularly the structural basis for its stereoselectivity and substrate specificity, we determined the X-ray crystal structure of LolT in its holo-form at 2.1 Å resolution. LolT is homodimeric with an overall fold similar to other aspartate aminotransferase-like fold type I (AAT-I) PLP-dependent enzymes. Electron density clearly reveals the covalently bound PLP cofactor, attached to K236 via a Schiff base (FIG. 11A). The orientation of PLP is fixed by an assortment of interactions, including a π-π stacking interaction with Y125, a salt bridge between pyridine nitrogen and D210, and a hydrogen bond between phenolic hydroxyl group and H213. Furthermore, the phosphate group of PLP is firmly anchored through extensive hydrogen bond interactions with T100, D233, and T304(B). Adjacent to PLP is a substrate binding pocket with a volume of ˜700 Å³, defined by both monomers and accessible through a ˜12 Å deep tunnel at the dimer interface (FIG. 11B). A highly flexible loop (as indicated by its B-factors) connecting residue P269(B) and T293(B), also comprising of a one-turn 3₁₀-helix, serves as a lid closing over the active site pocket. Molecular dynamics (MD) simulations results supported the flexible nature of this loop and its potential role in gating active site access (FIGS. 12A-12C).

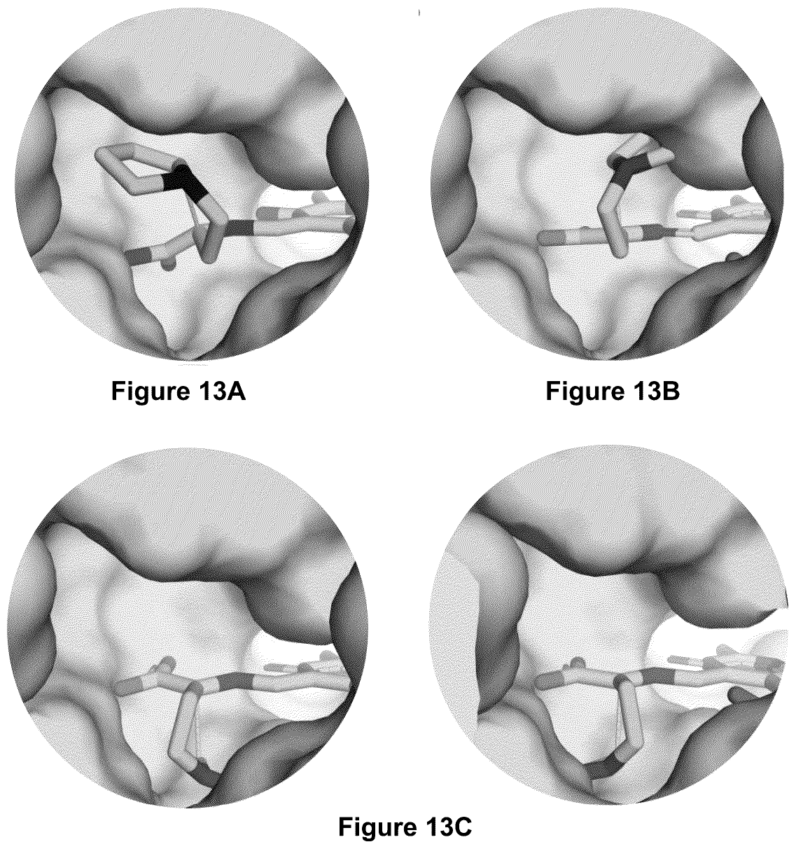

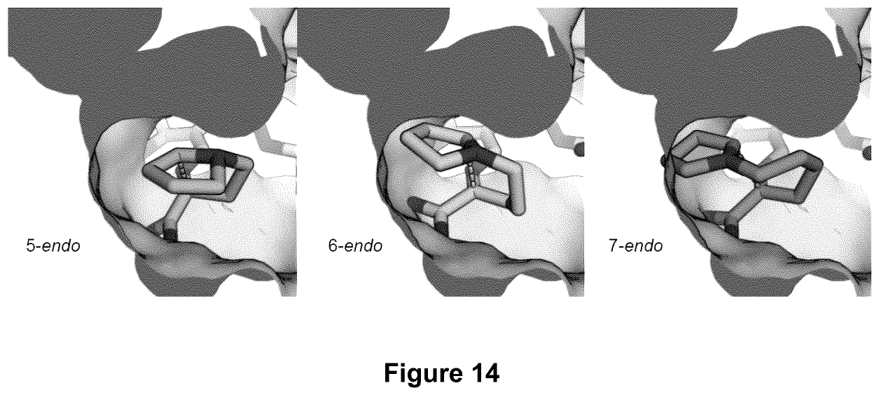

Despite numerous attempts, we were not able to obtain a co-crystal structure of LolT with either its native product 3, or the unreactive substrate mimic 2re, presumably due to low binding affinity. In order to gain mechanistic insights, we docked the DFT calculated transition state anti TS-1 with LolT. The energetically favorable conformation of the protein-ligand complex was obtained via four replicas of 1 μs MD simulations (FIG. 11C). The substrate α-carboxylate is recognized by LolT through a salt bridge with R438, and a hydrogen bond with S40. The cyclic iminium is making contact (3.6 Å) with W279(B) from the capping 3₁₀-helix through favorable cation-r interactions. On the si-face of PLP, liberated K236 is forming a hydrogen bond with the phosphate group of PLP. Its location and proximity to PLP-bound substrate render K236 the best candidate as the general base for initial deprotonation at Cα to trigger the cyclization reaction. Our docking-MD model also explains the stereochemical outcome. The si face of PLP is sterically hindered, such that the C—C bond formation prefers to occur at the re face causing inversion of configuration at Ca. Superimposing the syn cyclization transition state TS-2 with docked anti TS-1 reveals that TS-2 is in steric clash with Y125 (FIGS. 13A-13C), thereby LolT efficiently disfavors syn cyclization by sterically blocking the syn orientation of the iminium moiety. Fitting the respective transition state for 6-endo and 7-endo cyclization reveals that these transition states are not well accommodated in the active site pocket. In particular, the cyclic iminium side chain from 7-endo transition state is in steric clash with residues lining the pocket (FIG. 14), which supports our aforementioned hypothesis that the substrate scope is limited by active site pocket size, and also implies that the catalytic efficiency for 7-endo cyclization could be potentially improved by enlarging the active site pocket.

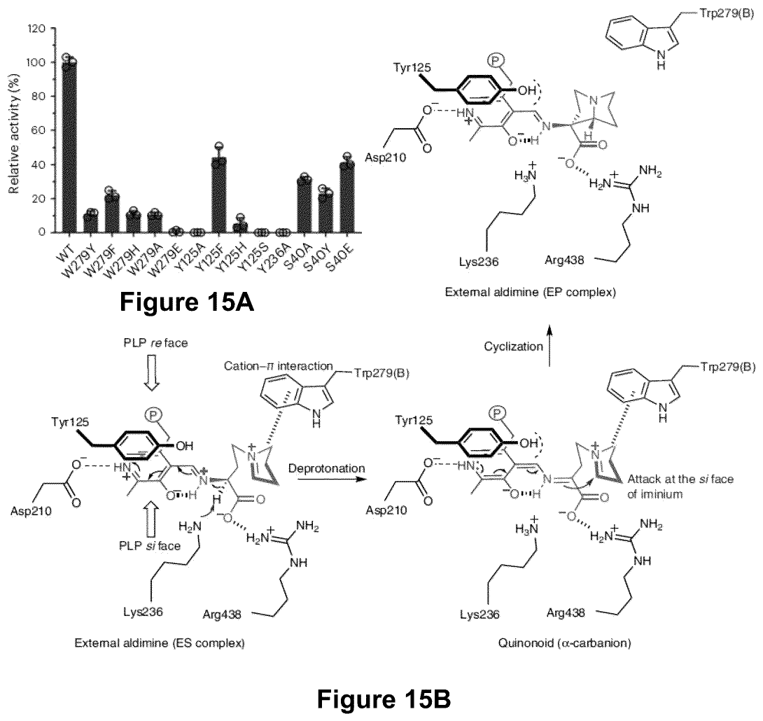

To examine the catalytic role of above-mentioned active site residues, we performed site-directed mutagenesis and assayed the enzyme activity using 2 as substrate (FIG. 15A). Alanine substitution at K236 abolished enzyme activity, confirming its essential role for activity. Mutations at Y125 revealed an indispensable role of the aromatic ring in conferring r-r interactions to bind and orient PLP for catalysis: Y125F and Y125H retained 45% and 5% activity, respectively; whereas Y125A and Y125S completely lost activity. Enzyme activity was also reduced significantly with various substitutions at W279(B), among which W279F retained the most of the activity (25% activity of WT), while W279E lost nearly 100-fold activity. These results confirmed the importance of the cation-r interaction made by W279(B), consistent with its strictly conserved nature among LolT orthologues. Besides, the fact that W279E mutant adversely impacted the catalysis suggests this cation-r interaction is not replaceable by a seemingly favorable ionic interaction. Lastly, mutations at S40 reduced the activity by 60%, in line with our docking-MD model that S40 plays a role to interact with substrate α-carboxylate group. The partially retained activity also suggests the major carboxylate recognition is from R438, a residue highly conserved in PLP-dependent enzymes. These structural insights together with mutagenesis data allowed us to propose a catalytic mechanism for LolT (FIGS. 15B-15C). Upon formation of the external aldimine, the pKa of Cα-H is lowered and K236 located at the si face of PLP acts as a general base to stereospecifically deprotonate L-configured α-amino acid, forming a Cα-carbanion (quinonoid intermediate), which then attacks at the si face of the iminium groups with concomitant inversion of configuration at Cα. To summarize, the enantio- and diastereo-selectivity were achieved by sterically blocking the undesired C—C bond forming trajectories.

### Expanded Synthetic Utility of LolT

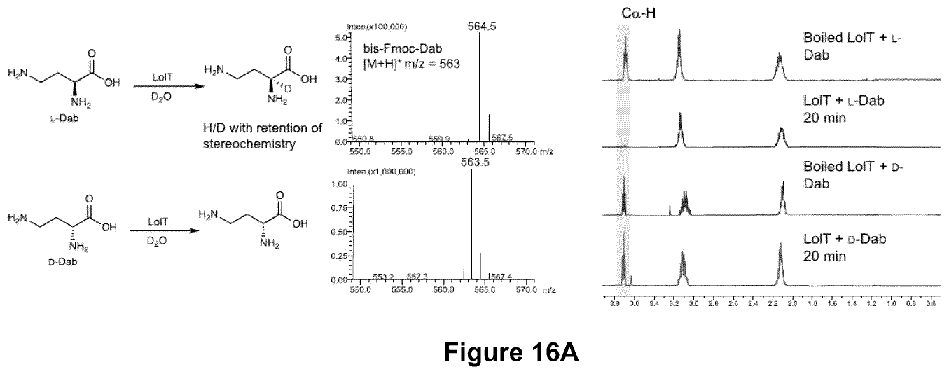

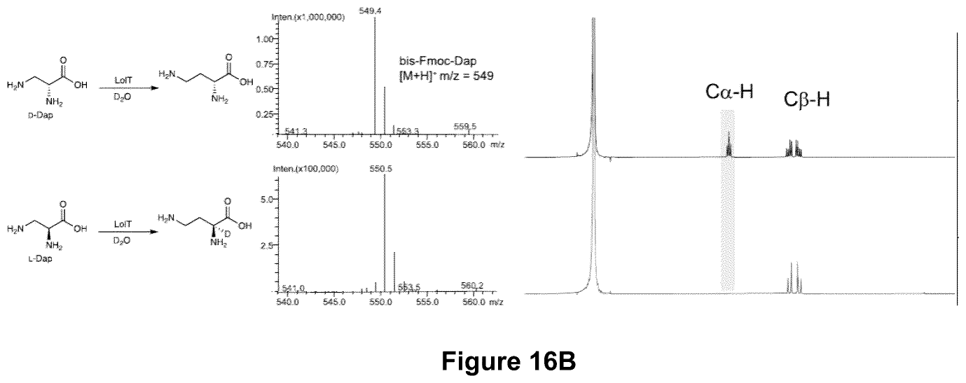

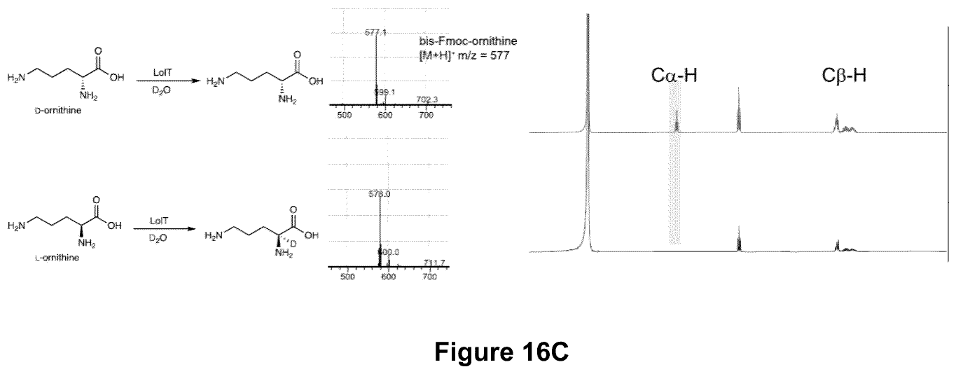

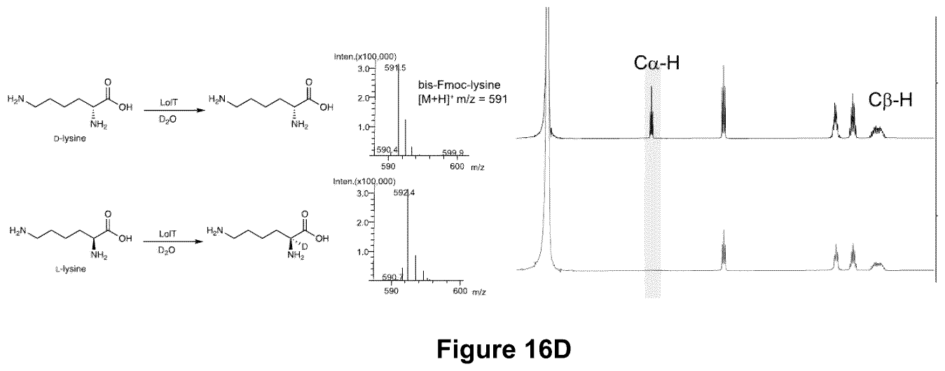

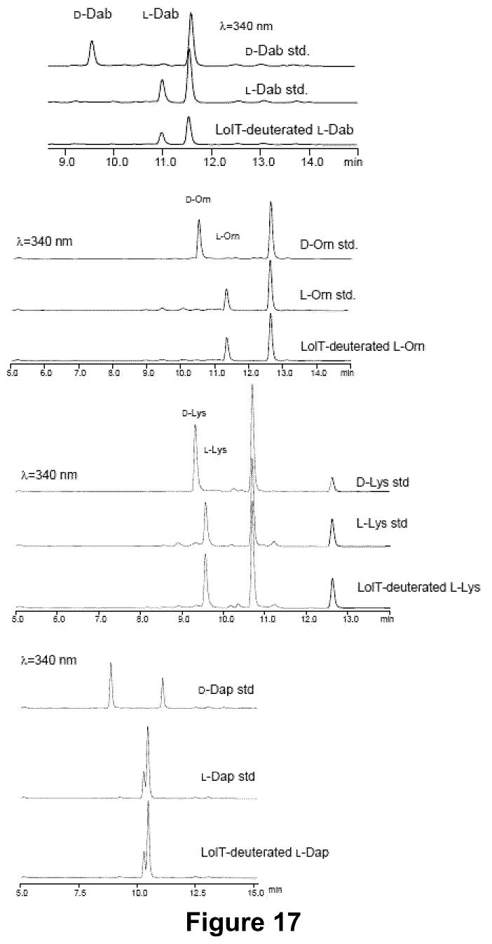

The crystal structure of LolT also suggests there are minimum interactions between LolT and substrate iminium side chain, except the discussed cation-π interaction. We hence reason that LolT may be promiscuous such that it will activate amino acids which resemble LolT's substrates but cannot undergo Mannich cyclization, e.g. 2,4-diaminobutanoic acid (Dab), a substructure of 2. Indeed, when L-Dab was incubated with LolT in D₂O, its Cα-H was rapidly washed out; while no H/D exchange occurred to D-Dab (FIGS. 16A-16D). Chiral analysis of deuterated L-Dab further showed that H/D exchange took place with complete retention of configuration (FIG. 17). Similar carbanion formation was also observed with other diamino acids, including 2,3-diaminopropanoic acid (Dap), ornithine and lysine. These results are in agreement with our proposed mechanism that LolT enantiospecifically deprotonates L-amino acid by the proposed general base K236. The same residue may also act as a general acid to facilitate reprotonation at Cα to ensure stereo-retention.

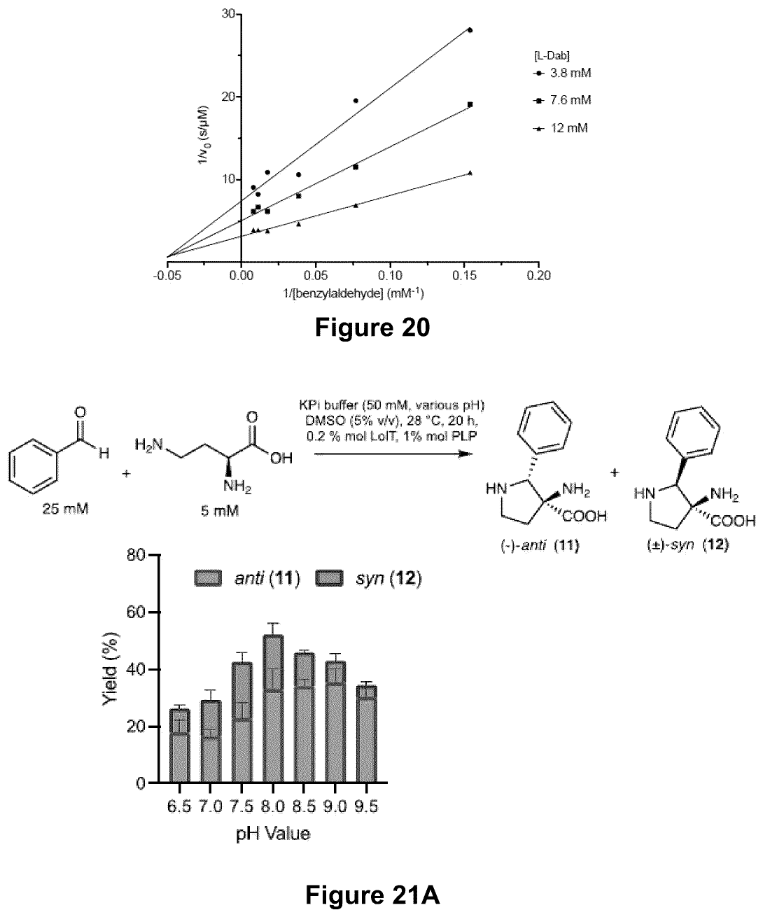

Inspired by this promiscuous deprotonase activity, we envisioned that carbanions generated from these diamino acids could be intercepted by imines that are formed in situ via condensation between their side chains and aldehydes (FIG. 18A). Preliminary screenings using various aldehydes and L-diamino acids confirmed this hypothesis (FIGS. 19A-19B). LolT accepted a broad range of aldehydes with L-Dab as the carbanion donor and catalyzed the corresponding 5-endo Mannich cyclization after imine formation to give pyrrolidine quaternary α-amino acids (FIG. 18B). Results from steady-state kinetic measurements indicate a ternary complex being formed during the reaction, consistent with a proposed PLP-bound imine intermediate (FIG. 20). In contrast, the aldehyde substrate scope gets narrower with L-ornithine as the carbanion donor. Only short-chain aliphatic aldehydes without α-substitution groups were accepted, giving the corresponding piperidine quaternary α-amino acids (6-endo-trig Mannich cyclization products). No Mannich cyclization reaction took place with L-lysine as the amino acid donor, consistent with our hypothesis that the active site pocket is not large enough to accommodate the transition state for 7-endo cyclization.

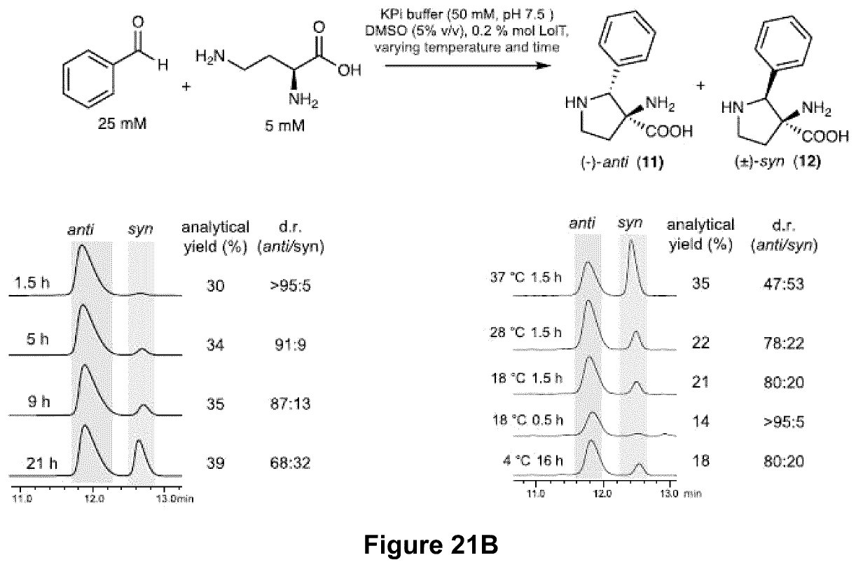

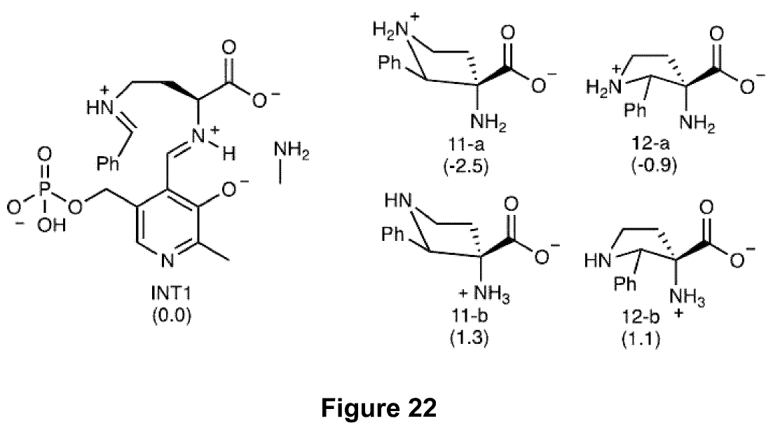

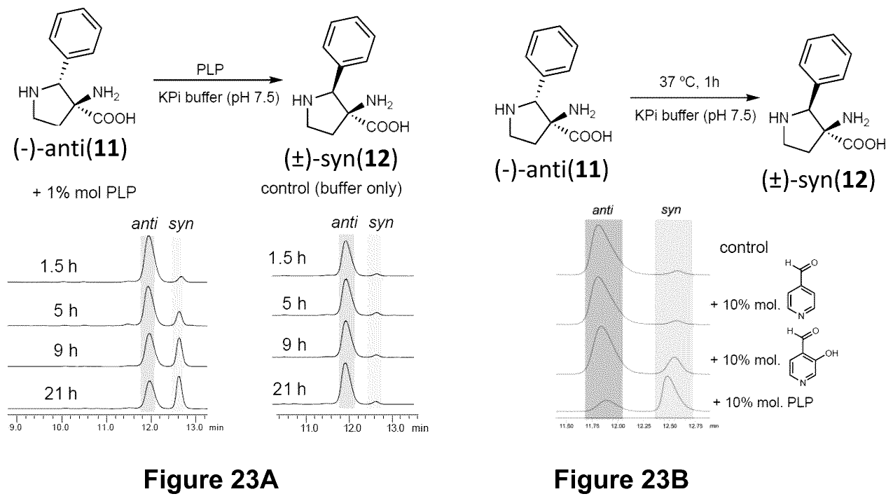

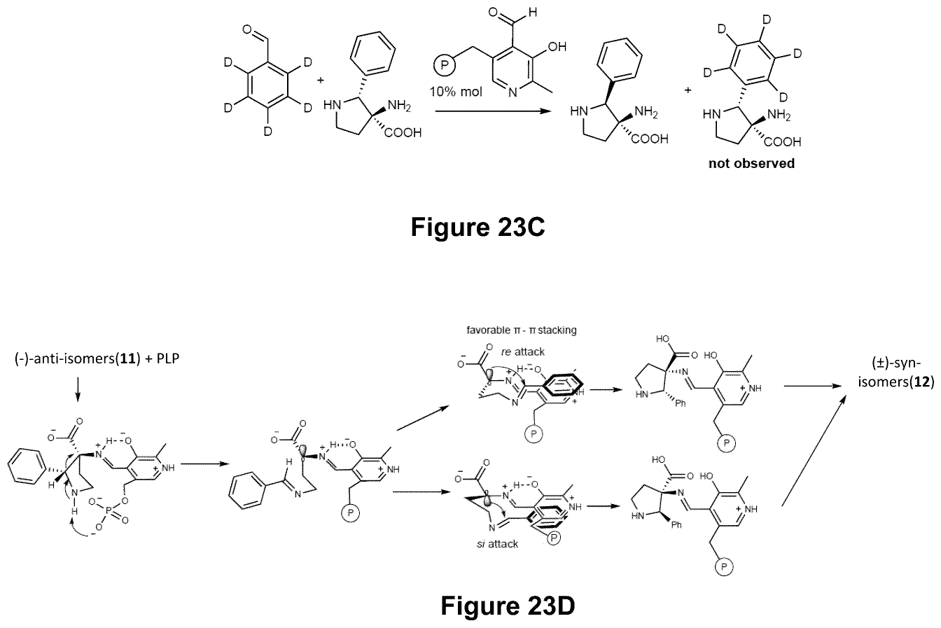

Regarding the stereoselectivity, LolT predominantly gave the anti-configured diastereoisomers with inverted Cα centers and high enantiomeric excess (FIG. 18B). This stereochemical outcome is in agreement with all other LolT-catalyzed cyclization reactions. Mechanistic investigations using benzaldehyde and L-Dab as model substrates showed that the products diastereoisomeric ratio (d.r.) is sensitive to the reaction conditions (FIGS. 21A-21B), a hallmark known for threonine aldolases. The anti-isomer 11 was favored at high pH and kinetically-controlled conditions (e.g. low temperature or short reaction time) (FIG. 22), whereas the racemate syn-isomer 12 was a nonenzymatic product that was slowly converted from 11 under the help of PLP (FIGS. 23A-23C). Overall, by using a mechanism-guided approach, we successfully expanded the synthetic utility of LolT to catalyze formal two-component Mannich cyclization reactions and provide access to a variety of pyrrolidine and piperidine-based quaternary α-amino acids bearing vicinal tertiary and quaternary stereocenters.

### CONCLUSIONS

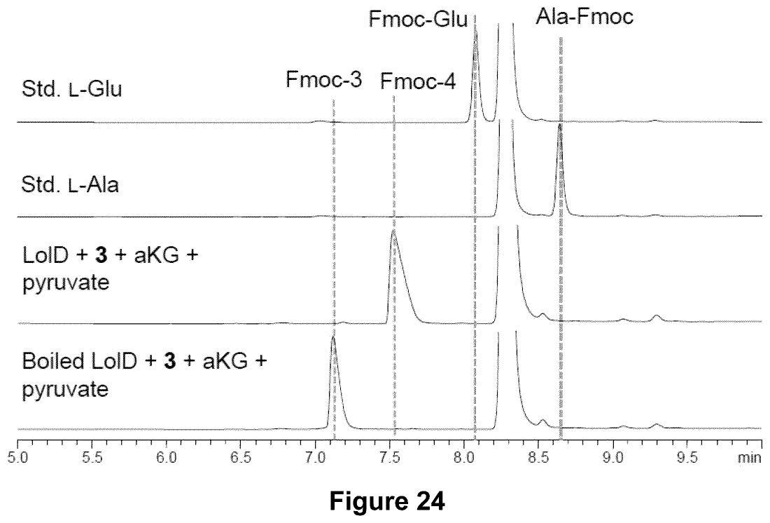

Our studies have elucidated the biosynthetic formation of pyrrolizidine core scaffold in loline alkaloids. In particular, we discovered two PLP-dependent enzymes: LolT catalyzes stereoselective Mannich cyclization reaction to yield a pyrrolizidine quaternary α-amino acid intermediate, while LolD subsequently catalyzes a stereo-retentive decarboxylation to give the desired 1-amino-pyrrolizidine scaffold. It is noteworthy to mention that LolD is distinct from the well-studied dialkylglycine decarboxylase (DGD). Although both PLP-dependent enzymes act on quaternary α-amino acids, LolD is a bona fide decarboxylase which only catalyze non-oxidative decarboxylation (FIG. 24), whereas DGD is a bi-functional enzyme catalyzing oxidative decarboxylation (i.e. tandem decarboxylation-transamination cascade).

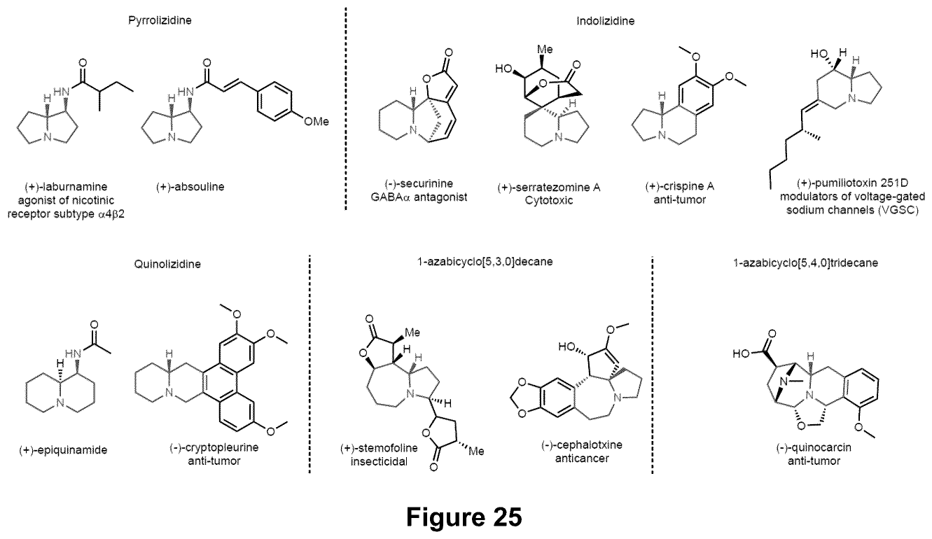

More importantly, we demonstrated that LolT is a versatile biocatalyst, accepting a wide range of substrates to make a myriad of quaternary α-amino acids with diverse heterocyclic backbones. In all scenarios, LolT shows high enantio- and diastereoselectivity, and predominantly makes the kinetically-controlled anti-diastereoisomers. Because these aza(bi)cyclic α,α-disubstituted amino acids are conformationally constrained building blocks playing vital roles in developing peptidomimetics and pharmaceuticals, LolT could be a useful biocatalytic tool for rapid access of these medicinally important molecules. In addition, the various aza(bi)cyclic skeletons made by LolT are also frameworks of a large number of structurally complex bioactive alkaloids (FIG. 25), yet a general method for asymmetric synthesis of these scaffolds is lacking. Thus, a chemoenzymatic approach based on LolT's Mannich cyclization activity could also be a general strategy for constructing diverse alkaloid scaffolds.

Additionally, our studies also illuminated the structural basis for LolT's stereoselectivity and substrate specificity. The proposed cyclization mechanism is reminiscent of another Cα-alkylating PLP-dependent cyclase, 1-aminocyclopropane-1-carboxylate synthase (ACCS), which catalyzes a S_{N}2-type 3-exo-tet cyclization to make cyclopropane quaternary α-amino acid. Despite this mechanistic similarity, LolT is evolutionarily unrelated to ACCS. Instead, phylogenetic analysis indicates LolT is closer to the CsdA family. Structural analysis by Dali server also shows the closest structural homologue to LolT is the carbon sulfoxide lyase Egt2 involved in ergothioneine biosynthesis. Notably, both Egt2 and CsdA family proteins catalyze β-elimination reactions to cleave C—S bonds (C—S lyase activity), which is distinct from LolT's function. This unexpected evolutionary relationship not only highlights that divergent evolution of PLP enzymes leads to catalytic versatility, but also reinforces the difficulty in accurately predicting the function of PLP enzymes solely based on sequence or structural similarity with known examples.

In summary, we have identified a PLP-dependent Mannich cyclase, LolT, from the biosynthetic pathway of Ioline alkaloids, and demonstrated its synthetic utility in stereoselective synthesis of diverse conformationally constrained aza(bi)cyclic quaternary α-amino acids. We also provided the structural basis and mechanistic insights to understand its C—C bond forming reactivity and stereoselectivity. Our work showcases how natural product biosynthesis facilitates biocatalytic innovations, and the insights gained from our study also laid the foundation to further develop new PLP-dependent biocatalysts.

### Methods

General Methods. Enzymatic reactions were monitored by LC-DAD-MS on a Shimadzu 2020 LC-MS (Phenomenex Kinetex, 1.7 μm, 2.0×100 mm, C18 column) using positive and negative mode electrospray ionization with a linear gradient of 2-98% MeCN—H₂O solvent system supplemented with 0.1% formic acid as additive. This instrument is also equipped with a photodiode-array detector (DAD) to facilitate quantitative analysis. Semi-preparative HPLC was carried out on a Shimadzu HPLC system, using a Cosmosil, C18 AR-II column (5.0 μm, 10 ID×250 mm, Shodex). Chemical reactions were monitored by thin layer chromatography (TLC) carried out on MilliporeSigma Aluminum TLC plates (silica gel 60 coated with F₂₅₄) using UV light for visualization and basic aqueous potassium permanganate as developing agent. NMR spectra were recorded on a Bruker Avance Ill 500 MHz NMR spectrometer. High-resolution mass spectra (HRMS) were acquired on an Agilent 1260 Infinity II HPLC-TOF system in electrospray ionization (ESI+) mode. Optical rotations were recorded on a Rudolph Research Laboratory AUTOPOL IV polarimeter. UV-Vis spectra were recorded on a Shimadzu 1601 spectrophotometer. Data fitting was performed using GraphPad Prism 9.

Chemicals. Boc-Dab-O^{t}Bu·HCl, Boc₂O, TBAF, tert-Butyldimethylsilyl chloride and (S)-tert-Butyl 5-amino-2-((tert-butoxycarbonyl)amino)pentanoate hydrochloride were purchased from Chem-Impex International, Inc. (S)-tert-Butyl 6-amino-2-((tert-butoxycarbonyl)amino)-hexanoate, (S)-2,4-diaminobutanoic acid, and pentane-1,5-diol were purchased from Ambeed, Inc. Isopropyl β-D-1-thiogalactopyranoside (IPTG) was purchased from Gold Biotechnology. Pyridoxine hydrochloride, sodium cyanoborohydride, Fmoc-Cl, o-phthaldialdehyde, and 6-mercaptohexanoic acid, and CM-SEPHADEX resin were purchased from Sigma-Aldrich. (Tert-butyldimethylsilyloxy)butanal was purchased from Aaron Chemicals LLC. (S)-2,3-diaminopropanoic acid, (R)-2,3-diaminopropanoic acid, Marfey's reagent, Na-(5-fluoro-2,4-dinitrophenyl)-L-leucinamide, and Na-(5-fluoro-2,4-dinitrophenyl)-D-leucinamide were purchased from TCI. Dess-Martin periodinane was purchased from Combi-block. TFA was purchased from Honeywell. All other chemicals and solvents were purchased from Fisher. Silica gel (56 Micron, S.A. 500 m²/g, P.V. 0.75 cc/g) from Alfa Aesar was used for flash column chromatography.

General DNA manipulation methods. E. coli XL-1 strain was used for cloning. DNA restriction enzymes were used as recommended by the manufacturer (New England Biolabs, NEB). Bacterial expression plasmids were ordered from Twist Biosciences. The codon-optimized DNA sequences are listed in FIG. 26. PCR was performed using Q5 High-Fidelity DNA Polymerase (NEB) with a Bio-Rad T100 thermocycler. DNA products were purified using commercial kits from Zymo Research. Single-point mutants were generated using the Gibson Assembly method (NEB).

Protein heterologous expression and purification. For P. expansum LolT (XP_016595153.1) and E. uncinate LolD (Q5MNI5), E. coli BL21(DE3) strain (Novagen) was used for heterologous expression. Briefly, E. coli transformants harboring the corresponding plasmids were grown overnight in LB medium containing 50 μg/mL kanamycin at 37° C. Each 1 litter fresh LB medium supplemented with 50 μg/mL kanamycin was inoculated with 5 mL of the overnight starting culture. The large cell culture growth was incubated at 37° C. and 250 rpm until optical density OD₆₀₀ reached 0.8. To induce protein expression, pyridoxin (10 μM, final concentration) and IPTG (200 μM, final concentration) were added to the culture medium, and the cells were left grown at 16° C. for 16 hours. Cells were harvested by centrifugation and resuspended in MPA buffer [50 mM K₂HPO₄ (pH 7.5), 10 mM imidazole, 300 mM NaCl, 5% glycerol] and lysed on ice by sonication. The cell lysate was centrifuged at 15,000 g for 30 min at 4° C. to remove the cellular debris. The cleared lysate supernatant was loaded onto Ni-NTA agarose resin (Qiagen) and the purification was carried out according to the manufacturer's instructions. Purified proteins were examined by (SDS-PAGE) and pure fractions were combined. Proteins were concentrated using 30 kDa Ultrafiltration centrifugal tubes (Amicon). For crystallization experiments, the concentrated proteins were supplemented with 1 mM pyridoxal 5′-phosphate (PLP) and subject to gel-filtration chromatography using a Superdex 200 26/60 column equilibrated in SEC buffer [50 mM HEPES (pH 7.25), 1 mM TCEP]. Protein concentration was determined by quantifying the PLP concentration (using e₃₈₈=6600 M⁻¹cm⁻¹) after releasing to the solution via base (0.2 N of NaOH) treatment. All mutants were expressed and purified using the same procedure as described for the wild-type.

For LolF expression, attempts were made to obtain soluble recombinant proteins but no successful expression condition was identified. Briefly, different LolF homologues were codon-optimized for E. coli expression including P. expansum LolF (XP_016595152.1) and E. uncinate LolF (Q5MN17). Both genes were ligated to modified pET28(a) vectors resulting in constructs harboring either an N-terminal His₆-tag, a C-terminal His₆-tag, an N-terminal GST-tag or an N-terminal MBP-tag. However, none of the expression plasmids gave soluble proteins using the expression condition described for LolT and LolD. Additionally, two uncharacterized LolF homologues were codon-optimized but no soluble protein was obtained upon overexpression in E. coli: Monosporascus sp. MG133 LolF (A0A4Q4VH07) and Heterodermia speciosa LolF (A0A8H3J612).

Protein crystallography. Crystals of holo-LolT were grown at 18° C. using the sitting drop vapor diffusion method in 3-μL drops containing a 1:1 mixture of the protein solution (10 mg/mL LolT in SEC buffer) and a reservoir solution (0.1 M sodium malonate, pH 5.2, 14% w/v PEG 3350). Plate-like crystals appeared after three days. All crystals were flash-frozen in liquid nitrogen after soaking in a cryoprotectant solution consisting of mother liquor supplemented with 25% (v/v) glycerol. All X-ray diffraction data were recorded at the Stanford Synchrotron Radiation Lightsource, beamline 9-2. Data reduction and integration was achieved with HKL3000. The structure was solved by molecular replacement using the program Phaser. Initial model was prepared through AlphaFold. The graphics program COOT was used for manual model building and refinement. PHENIX was used for crystallographic refinement and map calculation. Figures were prepared with Pymol.

Enzymatic assays for LolT and LolD. A typical reaction mixture (˜50 μl) includes ˜1 mM amino acid substrate in the assay buffer (50 mM K₂HPO₄, 50 μM PLP, pH 7.5). The reaction is initiated by addition of enzyme (final concentration ˜1 μM) and the reaction is incubated at either 28° C. or 37° C. The reaction is quenched by mixing with equal volume of acetonitrile. To analyze the product by LC-MS, the quenched reaction mixture was derivatized by Fmoc-Cl. Briefly, 15 μL of reaction mixture was mixed with 15 μL of sodium borate solution (1 M), and 15 μL of Fmoc-Cl stock solution (in acetonitrile) was added. The resulting mixture was incubated at 37° C. for 30 min and then injected to LC-MS. For steady-state kinetics, a time-course study was first performed to identify the linear region. Then the assays were performed in triplicate at various substrate concentrations. The product was quantified based on the peak area of derivatized sample and compared with standard curve. Besides Fmoc-Cl derivatization, o-phthaldialdehyde (OPA)-derivatization was also used. This derivatization was conducted by mixing 15 μL of reaction mixture with 15 μL of OPA reagent (50 mM o-phthaldialdehyde and 50 mM 6-mercaptohexanoic acid dissolved in methanol), and 5 μL of Na₂CO₃ (1 M). The resulting mixture was incubated at 37° C. for 30 min and then injected to LC-MS.

H/D exchange assay. A typical reaction mixture (˜500 μL) includes 10 μM LolT and 4 mM diamino acid substrate in assay buffer (12 mM Na₂HPO₄, 1.8 mM KH₂PO₄ pH 7.5 prepared in D₂O). Reaction was incubated at 28° C. and monitored by NMR and LC-MS (reaction mixture was derivatized with Fmoc-Cl as described above before MS analysis). Fully deuterated products were further derivatized with Marfey's reagent (TCL) for chiral analysis. Briefly, an 80 μL reaction mixture was mixed with 20 μL acetone solution of Marfey's reagent (50 mM) and 20 μL NaHCO₃ solution (1 M). The resulting mixture was incubated in a 42° C. water bath for 30 min. Next, 10 μL HCl (2M) was added to the derivatization mixture to quench the reaction. The resultant solution was analyzed on a Shimadzu LC-40 HPLC equipped with a Cosmosil, C18 AR-II analytical column. Derivatized amino acids were eluted with a linear gradient from 10% acetonitrile and to 40% acetonitrile over 20 min with a flow rate at 1 mL/min. All HPLC solvents (water and acetonitrile) were supplemented with 0.1% (v/v) formic acid.

Single-component Mannich cyclization assay for product isolation. A typical reaction mixture at preparation scale (˜20 mL) includes 5 μM LolT and 5 mM substrate in 50 mM KPi buffer [50 mM K₂HPO₄, 50 μM PLP, pH 7.5]. The reaction was incubated at 28° C. and monitored by LC-MS after Fmoc-Cl derivatization. The reaction was then quenched by 1 M HCl and the product was purified using cation exchange chromatography. To determine the enantiomeric excess (e.e.), isolated products were derivatized with Na-(5-fluoro-2,4-dinitrophenyl)-L-leucinamide and Na-(5-fluoro-2,4-dinitrophenyl)-D-leucinamide and separated on LC-MS.

Two-component Mannich cyclization assay. A typical reaction mixture at analytical scale (˜50 μL) includes 5 μM LolT, 5 mM diamino acid substrate, and 25 mM aldehyde substrates in 50 mM KPi buffer supplemented with 5% (v/v) DMSO. The reaction mixture was taken at different time points and derivatized with ortho-phthaldialdehyde (OPA) and 5-mercaptopentanoic acid before analysis on LC-MS. The pH value, reaction temperature, reaction time, and free PLP concentration (0.1%-10% mol) were optimized for selected reactions according to the analytic yield and diastereisomeric ratio (d.r.). Optimized reactions were scaled up to isolate the corresponding product for structural determination. To ease product isolation, the reaction mixtures were derivatized with Boc anhydride and isolated by reverse-phase chromatography. The isolated Boc-protected products were deprotected with TFA/CH₂Cl₂ and the isolation yields were calculated based on the Boc-protected form. To determine the enantiomeric excess (e.e.), isolated products were derivatized with Na-(5-fluoro-2,4-dinitrophenyl)-L-leucinamide and Na-(5-fluoro-2,4-dinitrophenyl)-D-leucinamide and separated on LC-MS. Briefly, 10 μL of amino acid product (10 mM in MeOH) was mixed with 10 μL of Na₂CO₃ (1 mM) and 10 μL of derivatizing reagent. The reaction was incubated at 42° C. for 1 h and quenched with 10 μL of 1 M HCl solution. The resulting mixture was analyzed by LC-MS.

DFT calculations. Conformational search was conducted using CREST of the XTB program. DFT were performed using Gaussian 16 program package. The structure of the lowest energy conformation of each species was submitted for geometry optimization at (oB97X-D/6-31G(d,p) level with CPCM solvation model for water and with the integration grid set to ultrafine level, followed by frequency calculation at the same theoretical level. All reported Gibbs free energies are for 298K and are after quasi-harmonic correction using the GoodVibes program.

Computation methods for docking and MD simulations. The transition state calculated using DFT methods was docked into the binding pocket of LolT using AutoDock Vina. For the best-scored binding pose, four replicas of 1000 ns MD simulations were further performed with AMBER 20 program package. Molecular dynamics simulations were prepared by the LEAP program in AMBERTOOLS 20 and performed with standard protocol as following: (1) The docked enzyme-transition state complex was energy-minimized with 5000 cycles. The first 2500 cycles were performed with the steepest descent algorithm but without the SHAKE algorithm activated. A positional restraint of 2 kcal mol⁻¹ Å⁻² was applied. (2) A 1 ns heating process was performed with activated SHAKE algorithm. The temperature was increased from 0 K to 300 K over 1 ns with the collision frequency of 5 ps⁻¹. Similar positional restraint of 2 kcal mol⁻¹ Å⁻² was applied; (3) A 2 ns equilibrium process was performed with periodic boundary for constant pressure (NPT) and a constant temperature (300K). Positional restraint of 2 kcal mol⁻¹ Å⁻² was also applied; (4) A 2 ns low-restraint equilibrium process was performed. And a positional restraint of 0.5 kcal mol⁻¹ Å⁻² was applied. (5) A 1000 ns production process was finally performed with the standard simulation conditions. For TS-LolT simulations, the bond forming distance of the transition state was constrained to the DFT optimized distance. CPPTRAJ program in AMBERTOOLS 20 was used for trajectory processing and analysis.

### Synthesis of (S)-2-amino-4-(pyrrolidin-1-yl)butanoic acid trifluoroacetate salt (2re)

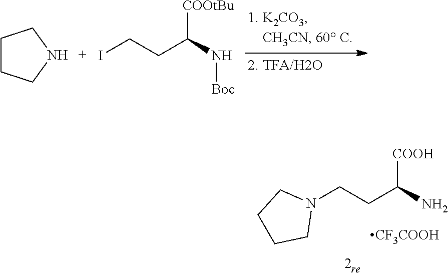

Pyrrolidine (79 mg, 1.02 mmol, 2.5 eq.), tert-butyl (S)-2-((tert-butoxycarbonyl)amino)-4-iodobutanoate (158 mg, 0.41 mmol, 1.0 eq.), and K₂CO₃ (212.4 mg, 1.53 mmol, 2.6 eq.) were dissolved in acetonitrile (20 mL). The reaction mixture was heated to 65° C. and stirred for 6 h. The solvent was then removed in vacuo, and the residue was subjected to flash column chromatography (DCM/MeOH/TEA=9:1:1) to afford a pale-yellow oil [R_{f}=0.45 (silica, 20% MeOH in DCM)]. This intermediate was then dissolved in ˜4 mL deprotection cocktail (DCM/TFA/H₂O=2:2:0.01). The reaction was stirred at room temperature for 4 h and concentrated by Rotary evaporation. The residue was taken in a minimal volume of TFA and precipitated with diethyl ether. The resulting solid was washed twice with ether and dried to afford the final product 2-H (52 mg, 50% yield for two steps). ¹H NMR (500 MHz, D₂O) δ 3.89 (dd, J=7.8, 5.5 Hz, 1H), 3.70 (ddt, J=11.4, 6.0, 3.0 Hz, 2H), 3.47 (ddd, J=12.8, 10.2, 6.2 Hz, 1H), 3.35 (ddd, J=12.8, 10.1, 5.3 Hz, 1H), 3.18-2.98 (m, 2H), 2.39-2.20 (m, 2H), 2.15 (td, J=6.8, 2.6, 2H), 2.01 (tt, J=6.7, 3.4 Hz, 2H). ¹³C NMR (125 MHz, D₂O) δ 172.5, 54.3, 54.0, 52.0, 51.5, 26.7, 22.59, 22.58. HRMS (m/z): [M+Na]⁺ calcd for C₈H₁₆N₂O₂Na⁺ 195.1104, found 195.1109.

### Synthesis of (S)-tert-butyl 4-((tert-butoxycarbonyl)(4-((tert-butyldimethylsilyl)oxy)butyl)amino)-2-((tert-butoxycarbonyl)amino)butanoate (2a)

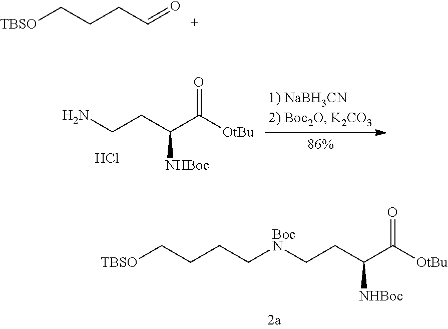

To a stirred solution of Boc-Dab-O^{t}Bu·HCl (500.0 mg, 1.61 mmol) in MeOH (65 mL) at 22° C., 4-(Tert-butyldimethylsilyloxy)butanal (325.2 mg, 1.61 mmol), AcOH (110 mg, 105 μl, 1.83 mmol), and NaBH₃CN (202.2 mg, 3.22 mmol) were sequentially added. The resulting mixture was stirred at 22° C. for 8 h and concentrated in vacuo. The residue was dissolved in THF/H₂O (16.5 ml, v/v=10/1). To this solution, K₂CO₃ (986.2 mg, 7.14 mmol) and Boc₂O (1.1160 g, 5.11 mmol) were added and stirred for 4 h at 22° C. Solvent was removed under in vacuo and the residue was subject to flash column chromatography and eluted with acetone/hexanes (1:10) to yield compound 2a (779.6 mg, 86% for the two steps) as a pale-yellow oil. 2a: R_{f}=0.45 (silica, acetone:hexanes 1:4); [α]23 D=−6 (c=1.0 in MeOH); ¹H NMR (500 MHz, CDCl₃): δ=5.26/5.08 (brs, 1H, NH), 3.81-4.33 (m, 1H), 3.58 (dd, J=6.2, 6.2 Hz, 2H), 2.91-3.44 (m, 4H), 1.98 (brs, 1H), 1.82 (brs, 1H), 1.21-1.62 (m, 29H), 0.86 (s, 9H), 0.02 (s, 6H) ppm; ¹³C NMR (126 MHz, CDCl₃): δ=171.6, 155.6, 82.0, 79.7, 79.6, 63.0, 52.3, 47.2, 43.6, 31.8, 30.7, 30.2, 28.6, 28.4, 28.1, 26.1, 25.2, 24.7, 18.4, −5.2 ppm; LC-MS (m/z): [M+H]⁺ calcd for C₂₈H₅₇N₂O₇Si⁺ 561.4, found 561.7.

### Synthesis of (S)-tert-butyl 4-((tert-butoxycarbonyl)(4-hydroxybutyl)amino)-2-((tert-butoxycarbonyl)amino)butanoate (2b)

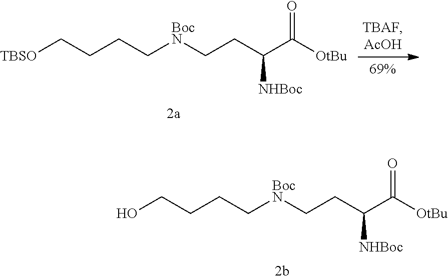 [SMILES: CC(C)(C)OC(=O)N[C@@H](CCN(CCCCO[Si](C)(C)C(C)(C)C)C(=O)OC(C)(C)C)C(=O)OC(C)(C)C ; CC(C)(C)OC(=O)N[C@@H](CCN(CCCCO)C(=O)OC(C)(C)C)C(=O)OC(C)(C)C]

To a stirred solution of compound 2a (779.6 mg, 1.39 mmol) in THF (14 mL) at 22° C., TBAF (4.2 ml, 4.2 mmol) and AcOH (262.2 mg, 250 ul, 4.37 mmol) were sequentially. The resulting mixture was stirred at 22° C. for 8 h and concentrated in vacuo. The residue was subject to flash column chromatography to yield compound 2b (428.3 mg, 69%) as a pale-yellow oil. 2b: R_{f}=0.40 (silica, acetone:hexanes v/v=1:2); [α]23 D=−6 (c=1.0 in MeOH); ¹H NMR (500 MHz, CDCl₃): δ=5.18 (brs, 1H, NH), 4.11 (dd, J=6.5, 6.5 Hz, 1H), 3.63 (dd, J=6.1, 6.1 Hz, 2H), 2.90-3.43 (m, 4H), 2.43 (s, 1H, OH), 1.89-2.11 (m, 1H), 1.71-1.89 (m, 1H), 1.21-1.64 (m, 29H) ppm; ¹³C NMR (126 MHz, CDCl₃): δ=171.6, 155.6, 82.1, 79.8, 62.4, 52.3, 47.0, 43.7, 31.7, 29.7, 28.5, 28.4, 28.1, 24.8 ppm; HRMS (m/z): [M+H]⁺ calcd for C₂₂H₄₃N₂O₇⁺ 447.3065, found 447.3062.

### Synthesis of compound (S)-tert-butyl 4-((tert-butoxycarbonyl)(4-oxobutyl)amino)-2-((tert-butoxycarbonyl)amino)butanoate (2c)

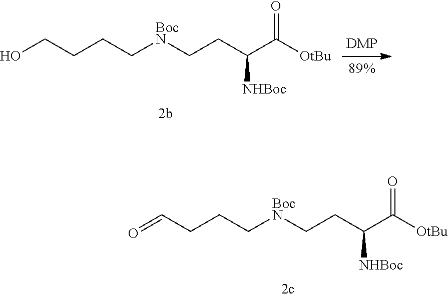 [SMILES: CC(C)(C)OC(=O)N[C@@H](CCN(CCCCO)C(=O)OC(C)(C)C)C(=O)OC(C)(C)C ; CC(C)(C)OC(=O)N[C@@H](CCN(CCCC=O)C(=O)OC(C)(C)C)C(=O)OC(C)(C)C]

To a solution of compound 2b (400.0 mg, 0.9 mmol) in CH₂Cl₂ (5 mL), Dess-Martin periodinane (570.5 mg, 1.35 mmol) was added. The reaction mixture was kept at 22° C. for 30 min and then directly subject to flash column chromatography (acetone/hexanes=1:4) to yield compound 2c (355.9 mg, 89%) as a pale-yellow oil. 2c: R_{f}=0.56 (silica, acetone:hexanes 1:2); [α]25 D=−7 (c=1.0 in MeOH); ¹H NMR (500 MHz, CDCl₃): δ=9.71 (s, 1H), 5.23/5.11 (brs, 1H, NH), 3.97-4.22 (m, 1H), 2.79-3.44 (m, 4H), 2.26-2.51 (m, 2H), 1.88-2.06 (m, 1H), 1.63-1.84 (m, 3H), 1.19-1.56 (m, 27H) ppm; ¹³C NMR (126 MHz, CDCl₃): δ=201.5, 171.4, 155.5, 155.4, 82.0, 79.9, 79.7, 52.2, 46.3, 43.6, 41.0, 31.6, 30.6, 28.5, 28.3, 28.0, 20.8 ppm; [M+Na]⁺ calcd for C₂₂H₄₀N₂O₇Na⁺ 467.2728, found 467.2770.

### Synthesis of compound (1S,7aR)-1-aminohexahydro-1H-pyrrolizine-1-carboylic acid (3)

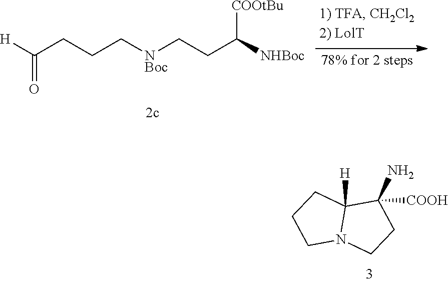

To a stirred solution of compound 2c (165.0 mg, 0.37 mmol) in CH₂Cl₂ (2 mL) at 0° C., TFA/H₂O (2.1 mL, v/v=20/1) was added. The reaction mixture was warmed up to room temperature (˜22° C.) and stirred for 12 h. Solvent was then removed in vacuo. The residue containing TFA was dissolved directly in concentrated phosphate buffer (3.7 ml, 500 mM K₂HPO₄, pH=7.5) to make the substrate stock solution (100 mM of deprotected 2c, calculated based on the starting material). The enzymatic reaction was set up in a 50 mL falcon tube by mixing the substrate stock solution with reaction buffer to yield a 30 mL reaction mixture [50 mM K₂HPO₄, 50 μM PLP, 5 mM substrate, pH 7.5]. The reaction was then initiated by addition of stock solutions of LolT (300 μM) to a final concentration at 5 μM. This enzymatic reaction was then incubated at 28° C. with gentle shaking (150 rpm) for 12 hrs. The reaction was quenched with 3 ml of 1 M HCl, and the crude mixture was directly subject to ion exchange chromatography using CM-SEPHADEX resin (from SIGMA). The enzymatic product was eluted using 0.5 M NH₃·H₂O. After removing the solvent in vacuo, pure compound 3 was obtained as a pale-yellow oil (19.8 mg, 78%). CCDC 2182351 contains the supplementary crystallographic data of 3·2HCl (EtOAc:MeOH 1:1). These data can be obtained free of charge from The Cambridge Crystallographic Data Centre. 3: R_{f}=0.23 (silica, CH₂Cl₂:MeOH:NH₃·H₂O 6:4:1.5); [α]23 D=+34 (c=0.5 in MeOH); ¹H NMR (500 MHz, CD₃OD): 5=3.78 (dd, J=8.0, 8.0 Hz, 1H), 3.65 (ddd, J=11.2, 8.9, 7.1 Hz, 1H), 3.50 (ddd, J=10.7, 6.4, 4.2 Hz, 1H), 3.24-3.39 (m, 2H), 2.99-3.13 (m, 1H), 2.64 (ddd, J=13.7, 8.3, 8.3 Hz, 1H), 2.05-2.19 (m, 2H), 2.00 (ddd, J=13.4, 6.7, 5.1 Hz, 1H), 1.80-1.96 (m, 2H) ppm; ¹³C NMR (126 MHz, CD₃OD): δ=177.1, 77.0, 68.8, 56.4, 54.6, 35.8, 28.9, 27.0 ppm; HRMS (m/z): [M+H]⁺ calcd for C₈H₁₅N₂O₂⁺ 171.1129, found 171.1132.

### Synthesis of compound (1S,7aR)-1-aminopyrrolizidine (4)

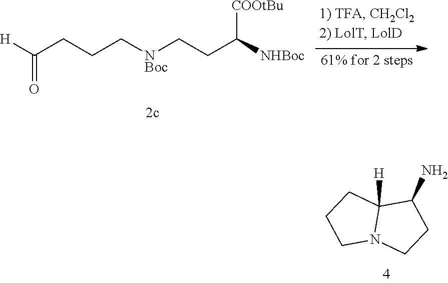

To a stirred solution of compound 2c (165.0 mg, 0.37 mmol) in CH₂Cl₂ (2 mL) at 0° C., deprotection cocktail solution TFA/H₂O (2.1 mL, v/v=20/1) was added. The reaction was warmed up to 22° C. and kept for 12 h. The solvent was removed in vacuo. The residue was dissolved in phosphate buffer (3.7 ml, 500 mM K₂HPO₃, pH=7.5) to make a substrate stock solution of deprotected 2c (at 100 mM, calculated based on the starting material). The enzymatic reaction was set up in a 50 mL falcon tube by mixing the substrate stock solution with reaction buffer to yield a 30 mL final reaction mixture [50 mM K₂HPO₄, 50 μM PLP, 5 mM substrate, pH 7.5]. The reaction was then initiated by addition of LolT and LolD with the final concentration at 5 μM and 10 μM, respectively. This enzymatic reaction was then incubated at 28° C. with gentle shaking (150 rpm) for 12 hrs. The reaction was quenched with 2 ml of 1 M HCl, and the crude mixture was directly subject to ion exchange chromatography using CM-SEPHADEX resin. The 1-amino-pyrrolizidine product was eluted with a gradient of ammonium hydroxide (1 M to 7M) and monitored by TLC. R_{f}=0.12 (silica, CH₂Cl₂:MeOH:NH₃·H₂O 6:4:1.5). Fractions containing the desired product were pooled. To stabilize the 1-amino-pyrrolizidine product, the pH of the combined solution was adjusted to 1 by using 1 M HCl. The solvent was then removed in vacuo. The resulting residue was dissolved in methanol and centrifuged to remove the insoluble ammonium hydrochloride. The supernatant containing 1-amino-pyrrolizidine hydrochloride (4·2HCl) was subject to gel filtration chromatography by using Sephadex LH20 resin (Sigma) with methanol as the mobile phase. Removing the solvent in vacuo yield pale-yellow oil (12.3 mg, 61%). 4·2HCl: [α]24 D=+15.3 (c=1.0 in MeOH), consistent with the literature value: [α]24 D=+12.2 (c=1.0 in H₂O); [α]20 D=+26.5 (c=0.7 in MeOH),¹ [α]20 D=+15.4 (c=1.2 in H₂O);^{1 1}H NMR (500 MHz, CD₃OD): δ=4.26-4.37 (m, 1H), 3.89 (q, J=6.5, 6.1 Hz, 1H), 3.61 (ddd, J=11.3, 6.5, 6.5 Hz, 1H), 3.33-3.46 (m, 1H), 3.21-3.33 (m, 1H), 2.67 (dq, J=12.9, 6.4 Hz, 1H), 2.34-2.46 (m, 1H), 2.24-2.32 (m, 1H), 2.16-2.24 (m, 1H), 2.02-2.16 (m, 2H) ppm; ¹³C NMR (126 MHz, CD₃OD): δ=71.3, 56.4, 55.6, 53.8, 30.8, 30.6, 25.9 ppm; HRMS (m/z): [M+H]⁺ calcd for C₇H₁₅N₂⁺ 127.1230, found 127.1235.

### Synthesis of (S)-tert-butyl 5-((tert-butoxycarbonyl)(4-((tert-butyldimethylsilyl)oxy)butyl)amino)-2-((tert-butoxycarbonyl)amino)pentanoate (4a)

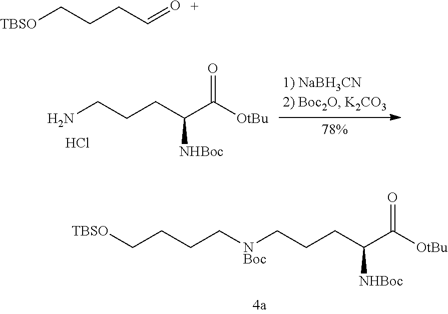

To a stirred solution of (S)-tert-Butyl 5-amino-2-((tert-butoxycarbonyl)amino)pentanoate hydrochloride (400.3 mg, 1.17 mmol) in MeOH (40 mL), 4-((tert-butyldimethylsilyl)oxy)butanal (236.6 mg, 1.17 mmol), HOAc (62.9 mg, 60 μl, 1.05 mmol), and NaBH₃CN (156.2 mg, 2.49 mmol) were sequentially added at 22° C. The resulting mixture was stirred at room temperature for 8 h and then concentrated in vacuo. The residue was dissolved in THF/H₂O (12 ml, v/v=10/1). To the stirred solution at 22° C., K₂CO₃ (663.4 mg, 4.80 mmol) and Boc₂O (785.7 mg, 3.60 mmol) were added. The reaction was kept at room temperature for 4 h and then concentrated in vacuo. The residue was subject to flash column chromatography (acetone/hexanes 1:10) to yield compound 4a (523.8 mg, 78% for the two steps) as a pale-yellow oil. 4a: R_{f}=0.67 (silica, acetone:hexanes 1:2); [α]23 D=−4 (c=2.0 in MeOH); ¹H NMR (500 MHz, CDCl₃): δ=5.03 (brs, 1H, NH), 3.88-4.29 (m, 1H), 3.59 (dd, J=6.2, 6.2 Hz, 2H), 2.95-3.32 (m, 4H), 1.65-1.80 (m, 1H), 1.26-1.63 (m, 34H), 0.87 (s, 9H), 0.02 (s, 6H) ppm; ¹³C NMR (126 MHz, CDCl₃): δ=171.8, 155.5, 155.4, 81.8, 79.6, 79.2, 62.9, 53.7, 46.9, 46.4, 30.4, 30.1, 28.5, 28.3, 28.0, 26.0, 25.2, 24.2, 18.3, −5.3 ppm; LC-MS (m/z): [M+H]⁺ calcd for C₂₉H₅₉N₂O₇Si⁺ 575.4, found 575.8.

### Synthesis of (S)-tert-butyl 5-((tert-butoxycarbonyl)(4-hydroxybutyl)amino)-2-((tert-butoxycarbonyl)amino)pentanoate (4b)

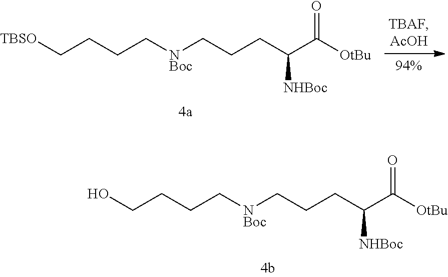 [SMILES: CC(C)(C)OC(=O)N[C@@H](CCCN(CCCCO[Si](C)(C)C(C)(C)C)C(=O)OC(C)(C)C)C(=O)OC(C)(C)C ; CC(C)(C)OC(=O)N[C@@H](CCCN(CCCCO)C(=O)OC(C)(C)C)C(=O)OC(C)(C)C]

To a stirred solution of 4a (523.8 mg, 0.91 mmol) in THF (9 mL), TBAF (2.7 ml, 2.7 mmol) and AcOH (167.8 mg, 160 μl, 2.8 mmol) were sequentially added at 22° C. The resulting mixture was stirred at room temperature for 8 h and then the THF was removed in vacuo. The residue was subject to flash column chromatography and the compound 4b is eluted with acetone/hexanes (1:2) as a pale-yellow oil (395.4 mg, 94%). 4b: R_{f}=0.44 (silica, acetone:hexanes 1:2); [α]23 D=−4 (c=1.0 in MeOH); ¹H NMR (500 MHz, CDCl₃): δ=5.07 (brs, 1H, NH), 4.12 (dd, J=5.8, 5.8 Hz, 1H), 3.62 (dd, J=6.2, 6.2 Hz, 2H), 3.00-3.30 (m, 4H), 2.52 (brs, 1H, OH), 1.63-1.88 (m, 1H), 1.46-1.63 (m, 7H), 1.24-1.46 (m, 27H) ppm; ¹³C NMR (126 MHz, CDCl₃): δ=171.9, 155.8, 155.5, 82.0, 79.7, 79.4, 62.4, 53.7, 46.8, 46.6, 30.5, 29.8, 28.5, 28.4, 28.1, 24.9, 24.5 ppm; HRMS (m/z): [M+H]⁺ calcd for C₂₃H₄₅N₂O₇⁺ 461.3221, found 461.3223.

### Synthesis of compound (S)-tert-butyl 5-((tert-butoxycarbonyl)(4-oxobutyl)amino)-2-((tert-butoxycarbonyl)amino)pentanoate (4c)

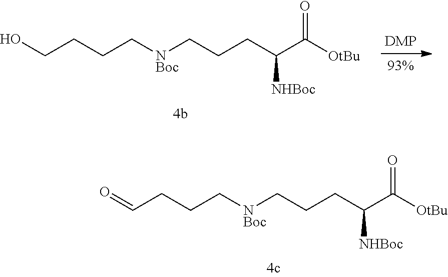 [SMILES: CC(C)(C)OC(=O)N[C@@H](CCCN(CCCCO)C(=O)OC(C)(C)C)C(=O)OC(C)(C)C ; CC(C)(C)OC(=O)N[C@@H](CCCN(CCCC=O)C(=O)OC(C)(C)C)C(=O)OC(C)(C)C]

To a stirred solution of compound 4b (395.4 mg, 0.86 mmol) in CH₂Cl₂ (8.5 mL) at 22° C., Dess-Martin periodinane (729.1 mg, 1.72 mmol) was and the reaction was stirred at room temperature for 30 min. The resulting mixture was directly subject to flash column chromatography (acetone/hexanes=1:4) to yield compound 4c (366.4 mg, 93%) as a pale-yellow oil. 4c: R_{f}=0.60 (silica, acetone:hexanes 1:2); [α]23 D=−5 (c=1.0 in MeOH); ¹H NMR (500 MHz, CDCl₃): δ=9.74 (s, 1H), 5.28-4.65 (m, 1H, NH), 3.79-4.36 (m, 1H), 2.80-3.48 (m, 4H), 2.42 (dd, J=7.2, 7.2 Hz, 2H), 1.76-1.96 (m, 2H), 1.62-1.76 (m, 1H), 1.48-1.62 (m, 3H), 1.17-1.48 (m, 27H) ppm; ¹³C NMR (126 MHz, CDCl₃): δ=201.7, 171.9, 155.6, 155.5, 82.0, 79.7, 79.7, 53.7, 46.7, 46.2, 41.1, 30.5, 28.5, 28.4, 28.1, 24.3, 21.0 ppm; [M+Na]⁺ calcd for C₂₃H₄₂N₂O₇Na⁺ 481.2884, found 481.2916.

### Synthesis of (8S,8aR)-8-aminooctahydroindolizine-8-carboxylic acid (5)

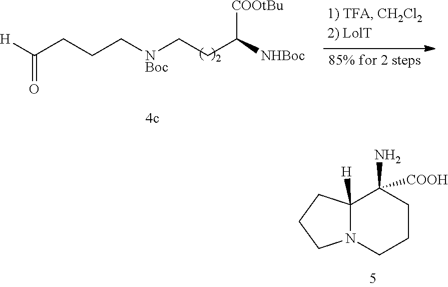

To a stirred solution of 4c (145.0 mg, 0.32 mmol) in CH₂Cl₂ (2 mL) at 0° C., TFA/H₂O (2.1 mL, v/v=20/1) was added. The reaction mixture was warmed up to 22° C. and kept for 12 h. The solvent was removed in vacuo and resulting residue was dissolved in phosphate buffer (3.2 mL, 500 mM K₂HPO₃, pH=7.5) to yield the substrate stock solution (100 mM calculated based on the starting material). The enzymatic reaction was set up in a 50 mL falcon tube by mixing the substrate stock solution with reaction buffer to yield final reaction mixture [50 mM K₂HPO₄, 50 μM PLP, 5 mM substrate, pH 7.5]. The reaction was then initiated by the addition of stock solutions of LolT (300 μM) to a final concentration at 5 μM. This enzymatic reaction (˜30 mL) was then incubated at 28° C. with gentle shaking (150 rpm) for 12 hrs. The reaction was quenched with 3 ml of 1 M HCl, and the crude mixture was directly subject to ion exchange chromatography using CM-SEPHADEX resin (Sigma-Aldrich). The enzymatic product was eluted with 0.5 M NH₃·H₂O. After removing the solvent in vacuo, pure compound 5 was obtained as a pale-yellow oil (23.2 mg, 85%). CCDC 2183481 contains the supplementary crystallographic data of 5·2HCl (EtOAc:MeOH 1:1). These data can be obtained free of charge from The Cambridge Crystallographic Data Centre. 5: R_{f}=0.23 (silica, CH₂Cl₂:MeOH:NH₃·H₂O 6:4:1.5); [α]23 D=+26 (c=1.0 in MeOH); ¹H NMR (500 MHz, CD₃OD): δ=3.70 (ddd, J=8.4, 8.4, 4.2 Hz, 1H), 3.61 (dd, J=11.8, 4.6 Hz, 1H), 2.98 (dd, J=10.8, 8.2 Hz, 2H), 2.85 (ddd, J=12.5, 12.5, 3.8 Hz, 1H), 2.22-2.42 (m, 1H), 1.95-2.17 (m, 4H), 1.79-1.95 (m, 2H), 1.55 (ddd, J=13.5, 13.5, 4.7 Hz, 1H) ppm; ¹³C NMR (126 MHz, CD₃OD): δ=179.2, 72.7, 59.3, 53.9, 52.1, 36.9, 25.7, 22.3, 20.7 ppm; HRMS (m/z): [M+Na]⁺ calcd for C₉H₁₇N₂O₂Na⁺ 207.1104, found 207.1109.

### Synthesis of tert-butyl N²,N⁶-bis(tert-butoxycarbonyl)-Ns-(4-((tert-butyldimethylsilyl)oxy)butyl)-L-lysinate (5a)

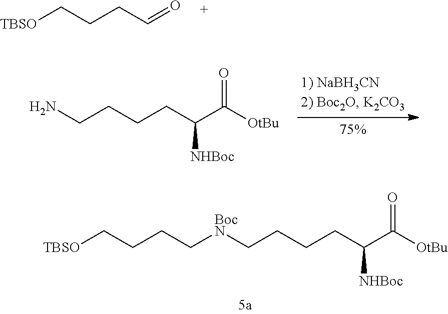

To a stirred solution of (S)-tert-butyl 6-amino-2-((tert-butoxycarbonyl)amino)hexanoate (305.6 mg, 1.01 mmol) in MeOH (40 mL) at 22° C., 4-((tert-butyldimethylsilyl)oxy)butanal (204.2 mg, 1.61 mmol), AcOH (62.9 mg, 60 μl, 1.05 mmol), and NaBH₃CN (188.4 mg, 3.0 mmol) were sequentially added. The resulting mixture was stirred at 22° C. for 8 h and concentrated in vacuo. The residue was dissolved in THF/H₂O (11 ml, v/v=10/1). To this solution, K₂CO₃ (552.8 mg, 4.0 mmol) and Boc₂O (873.0 g, 4.0 mmol) were added and stirred for 4 h at 22° C. Solvent was removed under in vacuo and the residue was subject to flash column chromatography and eluted with acetone/hexanes (1:10) to yield compound 5a (443.9 mg, 75% for the two steps) as a pale-yellow oil. 5a: R_{f}=0.51 (silica, acetone:hexanes 1:4); [α]23 D=−5 (c=1.0 in MeOH); ¹H NMR (500 MHz, CDCl₃): δ=5.07/5.00 (brs, 1H, NH), 4.11 (brs, 1H), 3.58 (dd, J=6.2, 6.2 Hz, 2H), 3.12 (brs, 4H), 1.66-1.83 (m, 1H), 1.17-1.66 (m, 36H), 0.86 (s, 9H), 0.01 (s, 6H) ppm; ¹³C NMR (126 MHz, CDCl₃): δ=172.1, 155.7, 155.5, 81.7, 79.6, 79.2, 63.0, 54.0, 47.1, 46.9, 46.6, 32.7, 30.2, 28.6, 28.4, 28.1, 26.2, 26.0, 25.3, 24.8, 22.6, 18.4, −5.2 ppm; HRMS (m/z): [M+Na]⁺ calcd for C₃₀H₆₀N₂O₇SiNa⁺ 611.4062, found 611.4064.

### Synthesis of tert-butyl N²,N⁶-bis(tert-butoxycarbonyl)-N⁶-(4-hydroxybutyl)-L-lysinate (5b)

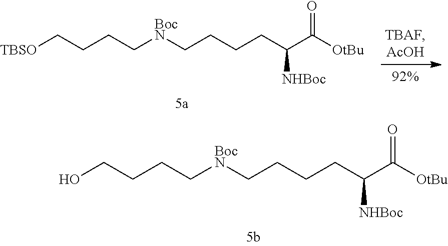 [SMILES: CC(C)(C)OC(=O)N[C@@H](CCCCN(CCCCO[Si](C)(C)C(C)(C)C)C(=O)OC(C)(C)C)C(=O)OC(C)(C)C ; CC(C)(C)OC(=O)N[C@@H](CCCCN(CCCCO)C(=O)OC(C)(C)C)C(=O)OC(C)(C)C]

To a stirred solution of 5a (443.9 mg, 0.75 mmol) in THF (7 mL) at 22° C., TBAF (3.5 ml, 3.5 mmol) and AcOH (220.3 mg, 210 ul, 4.37 mmol) were sequentially. The resulting mixture was stirred at 22° C. for 8 h and concentrated in vacuo. The residue was subject to flash column chromatography to yield compound 5b (327.5 mg, 92%) as a pale-yellow oil. 5b: R_{f}=0.40 (silica, acetone:hexanes v/v=1:2); [α]23 D=−5 (c=2.0 in MeOH); ¹H NMR (500 MHz, CDCl₃): δ=5.06/4.81 (brs, 1H, NH), 4.12 (brs, 1H), 3.63 (dd, J=6.1, 6.1 Hz, 2H), 2.90-3.36 (m, 4H), 2.33 (s, 1H, OH), 1.65-1.86 (m, 1H), 1.35-1.61 (m, 34H), 1.14-1.35 (m, 2H) ppm; ¹³C NMR (126 MHz, CDCl₃): δ=172.1, 155.8, 155.5, 81.8, 79.7, 79.4, 62.5, 54.0, 46.9, 32.7, 29.8, 28.6, 28.4, 28.1, 25.0, 22.6 ppm; HRMS (m/z): [M+Na]⁺ calcd for C₂₄H₄₆N₂O₇Na⁺ 497.3197, found 497.3206.

### Synthesis of compound tert-butyl N²,N⁶-bis(tert-butoxycarbonyl)-N⁶-(4-oxobutyl)-L-lysinate (5c)

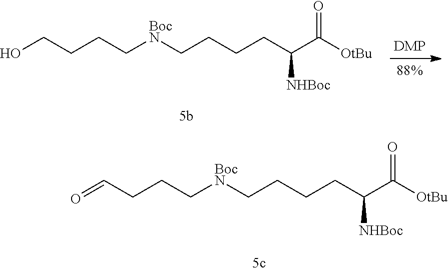 [SMILES: CC(C)(C)OC(=O)N[C@@H](CCCCN(CCCCO)C(=O)OC(C)(C)C)C(=O)OC(C)(C)C ; CC(C)(C)OC(=O)N[C@@H](CCCCN(CCCC=O)C(=O)OC(C)(C)C)C(=O)OC(C)(C)C]

To a solution of 5b (327.5 mg, 0.69 mmol) in CH₂Cl₂ (7 mL), Dess-Martin periodinane (585.4 mg, 1.38 mmol) was added. The reaction mixture was kept at 22° C. for 30 min and then directly subject to flash column chromatography (acetone/hexanes=1:4) to yield compound 5c (287.1 mg, 88%) as a pale-yellow oil. 5c: R_{f}=0.62 (silica, acetone:hexanes 1:2); [α]25 D=−6 (c=1.0 in MeOH); ¹H NMR (500 MHz, CDCl₃): δ=9.75 (s, 1H), 5.03/4.75 (brs, 1H, NH), 4.12 (q, J=7.2 Hz, 1H), 2.86-3.39 (m, 4H), 2.42 (dd, J=7.3, 7.3 Hz, 2H), 1.66-1.95 (m, 3H), 1.01-1.62 (m, 34H) ppm; ¹³C NMR (126 MHz, CDCl₃): δ=201.6, 171.9, 155.6, 155.4, 81.7, 79.5, 53.9, 46.7, 46.1, 41.0, 32.6, 28.4, 28.3, 28.0, 22.5, 20.9 ppm; HRMS (m/z): [M+Na]⁺ calcd for C₂₄H₄₄N₂O₇Na⁺ 495.3041, found 495.3050.

### Synthesis of compound (9S,9aR)-9-aminooctahydro-1H-pyrrolo[1,2-a]azepine-9-carboxylic acid (7)

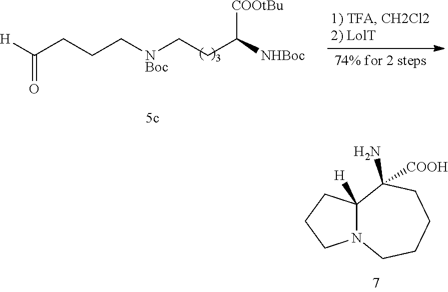

To a stirred solution of 5c (165.0 mg, 0.37 mmol) in CH₂Cl₂ (2 mL) at 0° C., TFA/H20 (2.1 mL, v/v=20/1) was added. The reaction mixture was warmed up to room temperature (˜22° C.) and stirred for 12 h. Solvent was then removed in vacuo. The residue containing TFA was dissolved directly in concentrated phosphate buffer (3.7 ml, 500 mM K₂HPO₄, pH=7.5) to make the substrate stock solution (100 mM of deprotected 5c, calculated based on the starting material). The enzymatic reaction was set up in a 50 mL falcon tube by mixing the substrate stock solution with reaction buffer to yield a 30 mL reaction mixture [50 mM K₂HPO₄, 50 μM PLP, 5 mM substrate, pH 7.5]. The reaction was then initiated by addition of stock solutions of LolT (300 μM) to a final concentration at 5 μM. This enzymatic reaction was then incubated at 28° C. with gentle shaking (150 rpm) for 12 hrs. The reaction was quenched with 3 ml of 1 M HCl, and the crude mixture was directly subject to ion exchange chromatography using CM-SEPHADEX resin. The enzymatic product was eluted using 0.5 M NH₃·H₂O. After removing the solvent in vacuo, 7 was obtained as a pale-yellow oil (22.0 mg, 74%). CCDC 2183482 contains the supplementary crystallographic data of 7·2HCl (EtOAc:MeOH 1:1). These data can be obtained free of charge from The Cambridge Crystallographic Data Centre. 7: R_{f}=0.23 (silica, CH₂Cl₂:MeOH:NH₃·H₂O 6:4:1.5); [α]23 D=+4 (c=1.0 in MeOH); ¹H NMR (500 MHz, CD₃OD): δ=3.63 (ddd, J=11.3, 6.7, 6.7 Hz, 1H), 3.43-3.58 (m, 2H), 3.12 (ddd, J=10.7, 6.8, 6.8 Hz, 2H), 2.31 (dddd, J=13.4, 8.2, 6.6, 6.6 Hz, 1H), 2.09 (ddd, J=14.5, 6.3, 3.8 Hz, 1H), 1.93-2.05 (m, 3H), 1.75-1.93 (m, 4H), 1.68 (ddd, J=13.6, 7.8, 4.6 Hz, 1H) ppm; ¹³C NMR (126 MHz, CD₃OD): δ=180.8, 72.1, 61.1, 58.6, 55.7, 43.3, 30.2, 27.2, 24.2, 21.9 ppm; HRMS (m/z): [M+H]⁺ calcd for C₁₀H₁₉N₂O₂⁺ 199.1441, found 199.1457.

### Synthesis of (S)-tert-butyl 4-((tert-butoxycarbonyl)(5-((tert-butyldimethylsilyl)oxy)pentyl)amino)-2-((tert-butoxycarbonyl)amino)butanoate (6a)

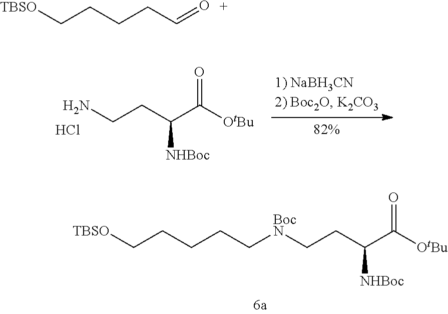

To a stirred solution of Boc-Dab-O^{t}Bu-HCl (300.0 mg, 0.965 mmol) in MeOH (40 mL), 5-((tert-butyldimethylsilyl)oxy)pentanal (208.5 mg, 0.965 mmol),² AcOH (62.9 mg, 60 μl, 1.05 mmol), and NaBH₃CN (125.6 mg, 2.0 mmol) were sequentially added at 22° C. The reaction mixture was stirred at room temperature for 8 h and then concentrated in vacuo. The residue was dissolved in THF/H₂O (10 ml, v/v=10/1). To this solution, K₂CO₃ (552.8 mg, 4.0 mmol) and Boc₂O (624.4 mg, 3.0 mmol) were added and stirred at 22° C. for another 4 h. After removing the solvent in vacuo, the residue was subject to flash column chromatography (acetone/hexanes=1:10) to yield compound 6a (464.8 mg, 82% for two steps) as a pale-yellow oil. 6a: R_{f}=0.65 (silica, acetone:hexanes 1:2); [α]23 D=−5 (c=1.0 in MeOH); ¹H NMR (500 MHz, CDCl₃): δ=5.28, 5.12 (brs, 1H, NH), 4.15 (q, J=7.5 Hz, 1H), 3.60 (dd, J=6.5, 6.5 Hz, 2H), 2.96-3.41 (m, 4H), 2.02 (brs, 1H), 1.85 (brs, 1H), 1.36-1.63 (m, 31H), 1.18-1.36 (m, 2H), 0.89 (s, 9H), 0.04 (s, 6H) ppm; ¹³C NMR (126 MHz, CDCl₃): δ=171.6, 155.5, 82.0, 79.8, 79.6, 63.2, 52.3, 47.3, 43.6, 32.7, 31.8, 30.8, 28.6, 28.4, 28.1, 26.1, 23.3, 18.5, −5.2 ppm; HRMS (m/z): [M+Na]⁺ calcd for C₂₉H₅₈N₂O₇SiNa⁺ 597.3905, found 597.3904.

### Synthesis of (S)-tert-butyl 4-((tert-butoxycarbonyl)(5-hydroxypentyl)amino)-2-((tert-butoxycarbonyl)amino)butanoate (6b)

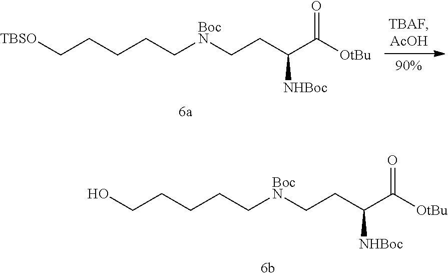 [SMILES: CC(C)(C)OC(=O)N[C@@H](CCN(CCCCCO[Si](C)(C)C(C)(C)C)C(=O)OC(C)(C)C)C(=O)OC(C)(C)C ; CC(C)(C)OC(=O)N[C@@H](CCN(CCCCCO)C(=O)OC(C)(C)C)C(=O)OC(C)(C)C]

To a stirred solution of 6a (464.8 mg, 0.81 mmol) in THF (8 mL), TBAF (2.4 mL, 2.4 mmol) and AcOH (151.0 mg, 144 μl, 2.5 mmol) were sequentially at 22° C. The resulting mixture was stirred at 22° C. for 8 h and then the solvent was removed in vacuo. The residue was subject to flash column chromatography (acetone/hexanes=1:2) to yield compound 6b (335.7 mg, 90%) as a pale-yellow oil. 6b: R_{f}=0.51 (silica, acetone:hexanes 1:2); [α]23 D=−3 (c=2.0 in MeOH); ¹H NMR (500 MHz, CDCl₃): δ=5.15 (brs, 1H, NH), 3.81-4.22 (m, 1H), 3.60 (dd, J=6.5, 6.5 Hz, 2H), 2.86-3.42 (m, 4H), 2.19 (s, 1H, OH), 1.90-2.09 (m, 1H), 1.66-1.90 (m, 1H), 1.35-1.66 (m, 31H), 1.15-1.35 (m, 2H) ppm; ¹³C NMR (126 MHz, CDCl₃): δ=171.6, 155.5, 82.1, 79.8, 79.7, 62.7, 52.4, 47.1, 43.7, 32.4, 31.7, 28.6, 28.4, 28.1, 23.0 ppm; HRMS (m/z): [M+H]⁺ calcd for C₂₃H₄₅N₂O₇⁺ 461.3221, found 461.3221.

### Synthesis of (S)-tert-butyl 4-((tert-butoxycarbonyl)(5-oxopentyl)amino)-2-((tert-butoxycarbonyl)amino)butanoate (6c)

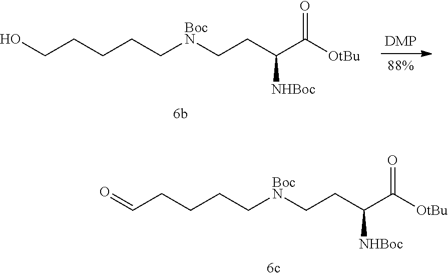 [SMILES: CC(C)(C)OC(=O)N[C@@H](CCN(CCCCCO)C(=O)OC(C)(C)C)C(=O)OC(C)(C)C ; CC(C)(C)OC(=O)N[C@@H](CCN(CCCCC=O)C(=O)OC(C)(C)C)C(=O)OC(C)(C)C]

To a stirred solution of compound 6b (335.7 mg, 0.73 mmol) in CH₂Cl₂ (7 mL) at 22° C., Dess-Martin periodinane (619.2 mg, 1.46 mmol) was. The reaction was kept at room temperature for 30 min and then directly subject to flash column chromatography (acetone/hexanes=1:4) to yield compound 6c (294.6 mg, 88%) as a pale-yellow oil. 6c: R_{f}=0.56 (silica, acetone:hexanes 1:2); [α]23 D=−5 (c=1.0 in MeOH); ¹H NMR (500 MHz, CDCl₃): δ=9.74 (s, 1H), 5.14 (brs, 1H, NH), 3.81-4.35 (m, 1H), 2.93-3.50 (m, 4H), 2.45 (dd, J=7.0, 7.0 Hz, 2H), 1.89-2.19 (m, 2H), 1.69-1.89 (m, 1H), 1.33-1.65 (m, 30H) ppm; ¹³C NMR (126 MHz, CDCl₃): δ=202.2, 171.6, 155.6, 155.4, 82.1, 79.8, 52.3, 46.8, 43.7, 43.6, 31.8, 31.0, 28.6, 28.4, 28.1, 27.8, 19.3 ppm; HRMS (m/z): [M+Na]⁺ calcd for C₂₃H₄₂N₂O₇Na⁺ 481.2884, found 481.2913.

### Synthesis of (1 S,8aR)-1-aminooctahydroindolizine-1-carboxylic acid (6)

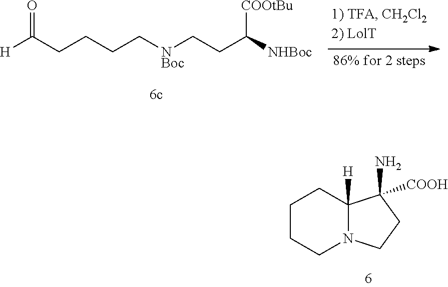

To a stirred solution of 6c (100.0 mg, 0.22 mmol) in CH₂Cl₂ (1.5 mL) at 0° C., TFA/H₂O (1.575 mL, v/v=20/1) was added. The reaction mixture was warmed up to 22° C. and kept for 12 h. The solvent was removed in vacuo and the resulting residue was dissolved in 500 mM K₂HPO₃ buffer (2.2 mL, PH=7.5) to yield the substrate stock solution (100 mM calculated based on the starting material 6c). The enzymatic reaction was set up in a 50 mL falcon tube by mixing the substrate stock solution with reaction buffer to yield a 30 mL reaction mixture [50 mM K₂HPO₄, 50 μM PLP, 5 mM substrate, pH 7.5]. The reaction was then initiated by the addition of stock solutions of LolT (300 μM) to a final concentration at 5 μM. This enzymatic reaction (˜30 mL) was then incubated at 28° C. with gentle shaking (150 rpm) for 12 hrs. The reaction was quenched with 3 ml of 1 M HCl, and the crude mixture was directly subject to ion exchange chromatography using CM-SEPHADEX resin. The enzymatic product was eluted with 0.5 M NH₃·H₂O. After removing the solvent in vacuo, pure compound 6 was obtained as a pale-yellow oil (23.8 mg, 86%). 6: R_{f}=0.25 (silica, CH₂Cl₂:MeOH:NH₃·H₂O 6:4:1.5); [α]23 D=+14 (c=1.0 in MeOH); ¹H NMR (500 MHz, CD₃OD): δ=3.36-3.51 (m, 2H), 2.84 (q, J=9.6 Hz, 1H), 2.39-2.64 (m, 2H), 1.95-2.07 (m, 1H), 1.83-1.95 (m, 2H), 1.78 (d, J=13.9 Hz, 1H), 1.63 (dddd, J=16.9, 13.0, 8.7, 4.1 Hz, 1H), 1.48 (qd, J=12.8, 3.2 Hz, 1H), 1.39 (dddd, J=16.8, 13.1, 8.0, 3.5 Hz, 1H) ppm; ¹³C NMR (126 MHz, CD₃OD): δ=177.0, 73.1, 66.4, 53.9, 53.0, 34.7, 25.8, 24.8, 23.8 ppm; HRMS (m/z): [M+Na]⁺ calcd for C₉H₁₇N₂O₂Na⁺ 207.1104, found 207.1109.

### Synthesis of (S)-tert-butyl 4-((tert-butoxycarbonyl)(6-hydroxyhexyl)amino)-2-((tert-butoxycarbonyl)amino)butanoate (7a)

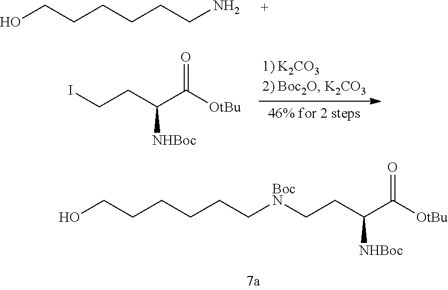

To a stirred solution of tert-butyl (S)-2-((tert-butoxycarbonyl)amino)-4-iodobutanoate (325.1 mg, 0.84 mmol) in CH₃CN (40 mL) at 22° C., K₂CO₃ (464.0 mg, 3.36 mmol) and 6-aminohexan-1-ol (297.0 mg, 2.53 mmol) were sequentially added. The resulting mixture was stirred at 60° C. for 4 h and then the solvent was removed in vacuo. The residue was subjected to flash column chromatography using hexanes/EtOAc/Et₂NH (4:1:1) as the mobile phase to yield a yellow oil product, R_{f}=0.63 (silica, DCM:MeOH 4:1). The product was dissolved in THF/H₂O (6.6 ml, v/v=10/1). To the stirred solution was added K₂CO₃ (464.0 mg, 3.36 mmol) and Boc₂O (550.8 mg, 2.52 mmol), and stirred at 22° C. for 4 h. The reaction was concentrated in vacuo and the residue was subject to flash column chromatography (acetone/hexanes=1:1) to yield compound 7a (183.4 mg, 46% for two steps) as a pale-yellow oil. 7a: R_{f}=0.50 (silica, acetone:hexanes 1:2); [α]23 D=−14 (c=1.0 in EtOH); ¹H NMR (500 MHz, CDCl₃): δ=5.41/5.13 (brs, 1H, NH), 3.73-4.31 (m, 1H), 3.52 (dd, J=6.6, 6.6 Hz, 2H), 2.81-3.39 (m, 4H), 2.61 (s, 1H), 1.84-2.06 (m, 1H), 1.61-1.83 (m, 1H), 1.03-1.61 (m, 33H) ppm; ¹³C NMR (126 MHz, CDCl₃): δ=171.5, 155.4, 81.9, 79.5, 62.3, 52.2, 47.2, 46.8, 43.5, 32.5, 31.5, 30.4, 28.4, 28.3, 28.0, 26.3, 25.3 ppm; HRMS (m/z): [M+H]⁺ calcd for C₂₄H₄₇N₂O₇⁺ 475.3378, found 475.3379.

### Synthesis of tert-butyl (S)-4-((tert-butoxycarbonyl)(6-oxohexyl)amino)-2-((tert-butoxycarbonyl)amino)butanoate (7b)

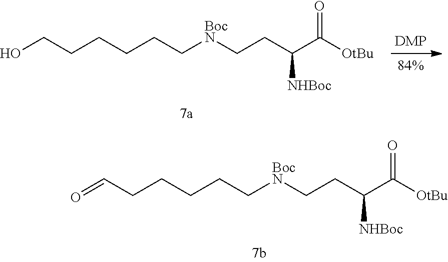 [SMILES: CC(C)(C)OC(=O)N[C@@H](CCN(CCCCCCO)C(=O)OC(C)(C)C)C(=O)OC(C)(C)C ; CC(C)(C)OC(=O)N[C@@H](CCN(CCCCCC=O)C(=O)OC(C)(C)C)C(=O)OC(C)(C)C]

To a stirred solution of 7a (183.4 mg, 0.386 mmol) in CH₂Cl₂ (4 mL), Dess-Martin periodinane (327.8 mg, 0.77 mmol) was added. The reaction was stirred at 22° C. for 30 min and then directly subject to flash column chromatography (acetone/hexanes=1:4) to yield compound 7b (153.2 mg, 84%) as a pale-yellow oil. 7b: R_{f}=0.56 (silica, EtOAc:hexanes 1:1); [α]25 D=−4 (c=1.0 in EtOH); ¹H NMR (500 MHz, CDCl₃): δ=9.68 (s, 1H), 5.25/5.12 (brs, 1H, NH), 3.75-4.27 (m, 1H), 2.89-3.46 (m, 4H), 2.36 (dd, J=7.4, 7.4 Hz, 2H), 1.85-2.07 (m, 1H), 1.65-1.85 (m, 1H), 1.57 (p, J=7.4 Hz, 2H), 1.28-1.50 (m, 27H), 1.11-1.26 (m, 2H) ppm; ¹³C NMR (126 MHz, CDCl₃): δ=202.4, 171.4, 155.5, 155.3, 81.9, 79.6, 52.2, 46.9, 43.8, 43.5, 31.6, 30.6, 28.4, 28.3, 28.0, 26.3, 21.8 ppm; HRMS (m/z): [M+H]⁺ calcd for C₂₄H₄₅N₂O₇⁺ 473.3221, found 473.3214.

### Synthesis of (1S,9aR)-1-aminooctahydro-1H-pyrrolo[1,2-a]azepine-1-carboxylic acid (8)

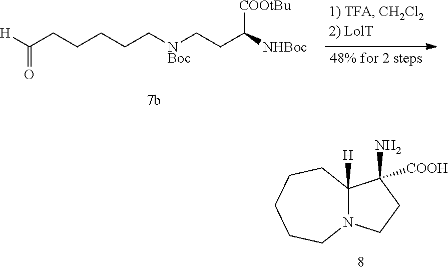

To a stirred solution of 7b (153.2 mg, 0.324 mmol) in CH₂Cl₂ (2 mL) at 0° C., TFA/H₂O (2.1 mL, v/v=20/1) was added. The reaction mixture was warmed up to 22° C. and kept at room temperature for 12 h. The solvent was removed in vacuo and the residue was dissolved in phosphate buffer(3.2 mL, 500 mM K₂HPO₃, pH=7.5) to make the substrate stock solution (100 mM calculated based on the starting material). The enzymatic reaction was set up in a 50 mL falcon tube by mixing the substrate stock solution with reaction buffer to yield a 30 mL final reaction mixture [50 mM K₂HPO₄, 50 μM PLP, 5 mM substrate, pH 7.5]. The reaction was then initiated by addition of stock solutions of LolT (300 μM) to a final concentration at 5 μM. This enzymatic reaction was then incubated at 28° C. with gentle shaking (150 rpm) for 12 hrs. The reaction was quenched with 3 ml of 1 M HCl, and the crude mixture was directly subject to ion exchange chromatography using CM-SEPHADEX resin (from SIGMA). The enzymatic product was eluted using 0.5 M NH₃·H₂O. After removing the solvent in vacuo, pure compound 8 was obtained as a pale-yellow oil (14.3 mg, 48%).8: R_{f}=0.35 (silica, CH₂Cl₂:MeOH:NH₃·H₂O 6:4:1.5); [α]23 D=+7 (c=1.0 in MeOH); ¹H NMR (500 MHz, CD₃OD): δ=3.63-3.77 (m, 1H), 3.49 (ddd, J=12.9, 6.4, 3.4 Hz, 1H), 3.22 (ddd, J=10.5, 10.5, 5.5 Hz, 1H), 3.04-3.15 (m, 1H), 2.99 (ddd, J=12.7, 9.0, 3.4 Hz, 1H), 2.40 (ddd, J=13.7, 8.5, 5.6 Hz, 1H), 1.95-2.10 (m, 2H), 1.80-1.94 (m, 3H), 1.68-1.80 (m, 3H), 1.54-1.68 (m, 1H) ppm; ¹³C NMR (126 MHz, CD₃OD): δ=177.8, 75.2, 67.8, 56.4, 55.4, 36.3, 26.7, 26.4, 26.1, 25.8 ppm; HRMS (m/z): [M+H]⁺ calcd for C₁₀H₁₉N₂O₂⁺ 199.1441, found 199.1445.

### Synthesis of (S)-tert-butyl 5-((tert-butoxycarbonyl)(5-((tert-butyldimethylsilyl)oxy)pentyl)amino)-2-((tert-butoxycarbonyl)amino)pentanoate (8a)

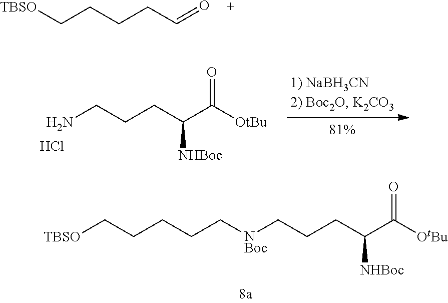

To a stirred solution of (S)-tert-Butyl 5-amino-2-((tert-butoxycarbonyl)amino)pentanoate hydrochloride (200.0 mg, 0.616 mmol) in MeOH (24 mL), 5-((tert-butyldimethylsilyl)oxy)pentanal² (126.2 mg, 0.583 mmol), AcOH (36.7 mg, 35 μl, 0.611 mmol), and NaBH₃CN (110.0 mg, 1.75 mmol) were sequentially added at 22° C. The resulting mixture was stirred for 8 h and then concentrated in vacuo. The residue was dissolved in THF/H₂O (5.5 ml, v/v=10/1). To the stirred solution at 22° C., K₂CO₃ (322.8 mg, 2.34 mmol) and Boc₂O (509.8 mg, 2.34 mmol) were added. The reaction was kept at room temperature for 4 h and then concentrated in vacuo. The residue was subject to flash column chromatography (acetone/hexanes 1:10) to yield compound 8a (295.5 mg, 81% for the two steps) as a pale-yellow oil. 8a: R_{f}=0.60 (silica, acetone:hexanes 1:2); [α]23 D=−5 (c=1.0 in MeOH); ¹H NMR (500 MHz, CDCl₃): δ=5.03 (s, 1H, NH), 4.13 (brs, 1H), 3.38-3.67 (m, 2H), 3.12 (brs, 4H), 1.62-1.80 (m, 1H), 1.32-1.62 (m, 33H), 1.14-1.32 (m, 3H), 0.72-1.01 (m, 9H), −0.12-0.11 (m, 6H) ppm; ¹³C NMR (126 MHz, CDCl₃): δ=171.9, 155.6, 155.5, 81.9, 79.7, 79.2, 63.2, 53.8, 47.1, 46.6, 32.7, 30.5, 28.6, 28.4, 28.1, 26.1, 24.5, 24.3, 23.3, 18.4, −5.2 ppm; HRMS (m/z): [M+H]⁺ calcd for C₃₀H₄₄NO₅Si⁺ 526.2983, found 526.2988.

### Synthesis of (S)-tert-butyl 5-((tert-butoxycarbonyl)(5-hydroxypentyl)amino)-2-((tert-butoxycarbonyl)amino)pentanoate (8b)

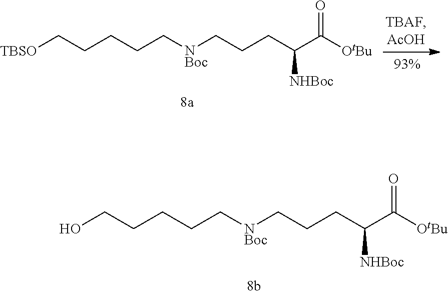 [SMILES: CC(C)(C)OC(=O)N[C@@H](CCCN(CCCCCO[Si](C)(C)C(C)(C)C)C(=O)OC(C)(C)C)C(=O)OC(C)(C)C ; CC(C)(C)OC(=O)N[C@@H](CCCN(CCCCCO)C(=O)OC(C)(C)C)C(=O)OC(C)(C)C]

To a stirred solution of 6a (295.5 mg, 0.50 mmol) in THF (5 mL), TBAF (1.5 mL, 1.5 mmol) and AcOH (94.4 mg, 90 μl, 1.57 mmol) were sequentially at 22° C. The resulting mixture was stirred at 22° C. for 8 h and then the solvent was removed in vacuo. The residue was subject to flash column chromatography (acetone/hexanes=1:2) to yield compound 8b (220.7 mg, 93%) as a pale-yellow oil. 8b: R_{f}=0.46 (silica, acetone:hexanes 1:2); [α]23 D=−4 (c=1.0 in MeOH); ¹H NMR (500 MHz, CDCl₃): δ=5.07 (brs, 1H, NH), 4.13 (brs, 1H), 3.61 (dd, J=6.5, 6.5 Hz, 2H), 2.90-3.31 (m, 4H), 2.11 (brs, 1H, OH), 1.65-1.86 (m, 1H), 1.36-1.65 (m, 33H), 1.24-1.36 (m, 2H) ppm; ¹³C NMR (126 MHz, CDCl₃): δ=171.9, 155.7, 155.5, 82.0, 79.8, 79.37, 62.7, 53.8, 47.0, 46.7, 32.4, 30.5, 28.6, 28.4, 28.1, 24.5, 23.0 ppm; HRMS (m/z): [M+Na]⁺ calcd for C₂₄H₄₆N₂O₇Na⁺ 497.3197, found 497.3206.

### Synthesis of (S)-tert-butyl 5-((tert-butoxycarbonyl)(5-oxopentyl)amino)-2-((tert-butoxycarbonyl)amino)pentanoate (8c)

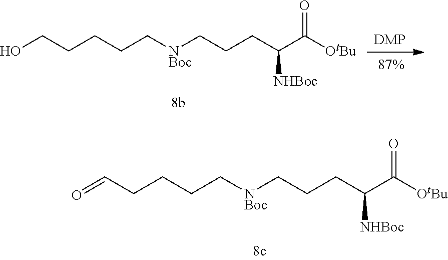 [SMILES: CC(C)(C)OC(=O)N[C@@H](CCCN(CCCCCO)C(=O)OC(C)(C)C)C(=O)OC(C)(C)C ; CC(C)(C)OC(=O)N[C@@H](CCCN(CCCCC=O)C(=O)OC(C)(C)C)C(=O)OC(C)(C)C]

To a stirred solution of compound 8b (220.7 mg, 0.465 mmol) in CH₂Cl₂ (5 mL) at 22° C., Dess-Martin periodinane (394.5 mg, 0.93 mmol) was. The reaction was kept at room temperature for 30 min and then directly subject to flash column chromatography (acetone/hexanes=1:4) to yield compound 8c (191.2 mg, 87%) as a pale-yellow oil. 8c: R_{f}=0.56 (silica, acetone:hexanes 1:2); [α]23 D=−7 (c=1.0 in MeOH); ¹H NMR (500 MHz, CDCl₃): δ=9.74 (s, 1H), 4.66-5.20 (m, 1H, NH), 3.81-4.29 (m, 1H), 3.14 (brs, 4H), 2.44 (dd, J=7.0, 7.0 Hz, 2H), 1.65-1.86 (m, 1H), 1.14-1.65 (m, 33H) ppm; ¹³C NMR (126 MHz, CDCl₃): δ=202.4, 202.1, 171.9, 155.6, 155.5, 82.0, 79.7, 79.5, 53.7, 46.6, 43.6, 30.5, 28.5, 28.4, 28.2, 28.1, 27. 7, 24.5, 24.3, 19.3 ppm; HRMS (m/z): [M+Na]⁺ calcd for C₂₄H₄₄N₂O₇Na⁺ 495.3041, found 495.3044.

### Synthesis of (1S,9aR)-1-aminooctahydro-2H-quinolizine-1-carboxylic acid (9)

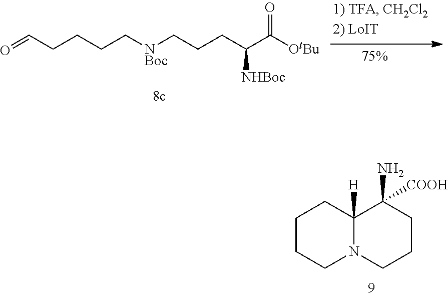 [SMILES: CC(C)(C)OC(=O)N[C@@H](CCCN(CCCCC=O)C(=O)OC(C)(C)C)C(=O)OC(C)(C)C ; N[C@@]1(C(=O)O)CCCN2CCCC[C@@H]21]

To a stirred solution of 8c (191.2 mg, 0.40 mmol) in CH₂Cl₂ (2 mL) at 0° C., TFA/H₂O (2.1 mL, v/v=20/1) was added. The reaction mixture was warmed up to 22° C. and kept for 12 h. The solvent was removed in vacuo and the resulting residue was dissolved in 500 mM K₂HPO₃ buffer (2.2 mL, PH=7.5) to yield the substrate stock solution (100 mM calculated based on the starting material 8c). The enzymatic reaction was set up in a 50 mL falcon tube by mixing the substrate stock solution with reaction buffer to yield a 20 mL reaction mixture [50 mM K₂HPO₄, 50 μM PLP, 5 mM substrate, pH 7.5]. The reaction was then initiated by the addition of stock solutions of LolT (300 μM) to a final concentration at 5 μM. This enzymatic reaction (˜20 mL) was then incubated at 28° C. with gentle shaking (150 rpm) for 12 hrs. The reaction was quenched with 2 ml of 1 M HCl, and the crude mixture was directly subject to ion exchange chromatography using CM-SEPHADEX resin. The enzymatic product was eluted with 0.5 M NH₃·H₂O. After removing the solvent in vacuo, pure compound 9 was obtained as a pale-yellow oil (15.0 mg, 75%). 9: CCDC 2190686 contains the supplementary crystallographic data of 9·2HCl (EtOAc:MeOH 1:1). These data can be obtained free of charge from The Cambridge Crystallographic Data Centre. R_{f}=0.30 (silica, CH₂Cl₂:MeOH:NH₃·H₂O 6:4:1.5); [α]23 D=+9 (c=1.0 in MeOH); ¹H NMR (500 MHz, CD₃OD): δ=3.41-3.51 (m, 1H), 3.32-3.40 (m, 1H), 2.92 (ddd, J=12.9, 12.9, 3.7 Hz, 1H), 2.78-2.88 (m, 2H), 2.13-2.31 (m, 2H), 1.94-2.02 (m, 1H), 1.83-1.94 (m, 2H), 1.75-1.83 (m, 1H), 1.68-1.75 (m, 1H), 1.56-1.66 (m, 2H), 1.44-1.56 (m, 1H) ppm; ¹³C NMR (126 MHz, CD₃OD): δ=179.2, 70.3, 59.8, 56.2, 56.0, 36.1, 25.3, 24.6, 23.3, 21.3 ppm; HRMS (m/z): [M+H]⁺ calcd for C₁₀H₁₉N₂O₂⁺ 199.1441, found 199.1447.

### Synthesis of (S)-tert-butyl 5-((tert-butoxycarbonyl)(6-((tert-butyldimethylsilyl)oxy)hexyl)amino)-2-((tert-butoxycarbonyl)amino)pentanoate (9a)

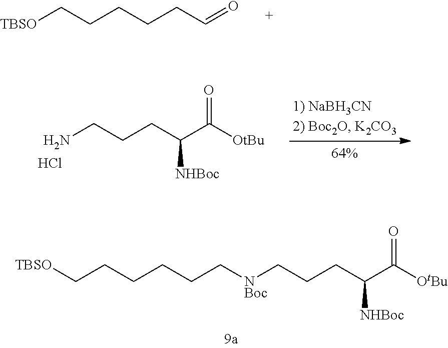 [SMILES: CC(C)(C)[Si](C)(C)OCCCCCC=O ; Cl ; CC(C)(C)OC(=O)N[C@@H](CCCN)C(=O)OC(C)(C)C ; CC(C)(C)OC(=O)N[C@@H](CCCN(CCCCCCO[Si](C)(C)C(C)(C)C)C(=O)OC(C)(C)C)C(=O)OC(C)(C)C]

To a stirred solution of (S)-tert-Butyl 5-amino-2-((tert-butoxycarbonyl)amino)pentanoate hydrochloride (186.0 mg, 0.57 mmol) in MeOH (25 mL), 6-((tert-butyldimethylsilyl)oxy)hexanal³ (123.0 mg, 0.54 mmol), AcOH (36.7 mg, 35 μl, 0.611 mmol), and NaBH₃CN (101.7 mg, 1.62 mmol) were sequentially added at 22° C. The resulting mixture was stirred at room temperature for 8 h and then concentrated in vacuo. The residue was dissolved in THF/H₂O (5.5 ml, v/v=10/1). To the stirred solution at 22° C., K₂CO₃ (322.8 mg, 2.34 mmol) and Boc₂O (509.8 mg, 2.34 mmol) were added. The reaction was kept at room temperature for 4 h and then concentrated in vacuo. The residue was subject to flash column chromatography (acetone/hexanes 1:10) to yield compound 9a (221.3 mg, 64% for the two steps) as a pale-yellow oil. 9a: R_{f}=0.72 (silica, acetone:hexanes 1:2); [α]23 D=−5 (c=1.0 in MeOH); ¹H NMR (500 MHz, CDCl₃): δ=4.88-5.15 (m, 1H, NH), 3.88-4.26 (m, 1H), 3.58 (dd, J=6.6, 6.6 Hz, 2H), 3.00-3.29 (m, 4H), 1.74 (brs, 1H), 1.36-1.66 (m, 33H), 1.14-1.36 (m, 5H), 0.87 (s, 9H), 0.03 (s, 6H) ppm; ¹³C NMR (126 MHz, CDCl₃): δ=172.0, 155.7, 155.5, 82.0, 79.7, 79.2, 63.3, 53.8, 47.2, 46.6, 33.0, 31.7, 30.5, 28.6, 28.4, 28.1, 26.8, 26.1, 25.8, 24.4, 18.5, −5.2 ppm; [M+Na]⁺ calcd for C₃₁H₆₂N₂O₇Na⁺ 625.4218, found 625.4222.

### Synthesis of (S)-tert-butyl 5-((tert-butoxycarbonyl)(6-hydroxyhexyl)amino)-2-((tert-butoxycarbonyl)amino)pentanoate (9b)

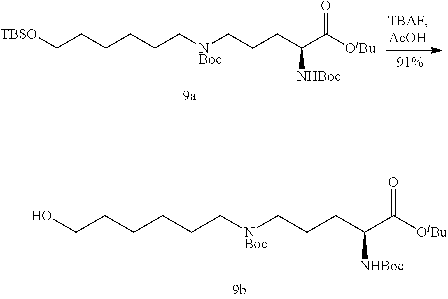 [SMILES: CC(C)(C)OC(=O)N[C@@H](CCCN(CCCCCCO[Si](C)(C)C(C)(C)C)C(=O)OC(C)(C)C)C(=O)OC(C)(C)C ; CC(C)(C)OC(=O)N[C@@H](CCCN(CCCCCCO)C(=O)OC(C)(C)C)C(=O)OC(C)(C)C]

To a stirred solution of 9a (221.3 mg, 0.367 mmol) in THF (4 mL), TBAF (1.1 mL, 1.1 mmol) and AcOH (69.2 mg, 66 μl, 1.15 mmol) were sequentially at 22° C. The resulting mixture was stirred at 22° C. for 8 h and then the solvent was removed in vacuo. The residue was subject to flash column chromatography (acetone/hexanes=1:2) to yield compound 9b (163.8 mg, 91%) as a pale-yellow oil. 9b: R_{f}=0.55 (silica, acetone:hexanes 1:2); [α]23 D=−5 (c=1.0 in MeOH); ¹H NMR (500 MHz, CDCl₃): δ=5.06 (brs, 1H, NH), 4.15 (brs, 1H), 3.62 (dd, J=6.5, 6.5 Hz, 2H), 2.90-3.35 (m, 4H), 1.90 (s, 1H, OH), 1.64-1.78 (m, 1H), 1.08-1.64 (m, 38H) ppm; ¹³C NMR (126 MHz, CDCl₃): δ=172.0, 155.8, 155.5, 82.0, 79.8, 79.3, 62.7, 53.8, 46.9, 46.6, 32.7, 30.5, 28.6, 28.4, 28.1, 26.5, 25.4, 24.5 ppm; [M+Na]⁺ calcd for C₂₅H₄₈N₂O₇Na⁺ 511.3354, found 511.3355.

### Synthesis of (S)-tert-butyl 5-((tert-butoxycarbonyl)(6-oxohexyl)amino)-2-((tert-butoxycarbonyl)amino)pentanoate (9c)

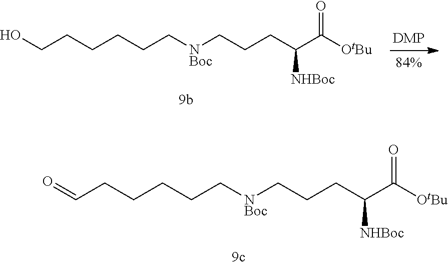 [SMILES: CC(C)(C)OC(=O)N[C@@H](CCCN(CCCCCCO)C(=O)OC(C)(C)C)C(=O)OC(C)(C)C ; CC(C)(C)OC(=O)N[C@@H](CCCN(CCCCCC=O)C(=O)OC(C)(C)C)C(=O)OC(C)(C)C]

To a stirred solution of 9b (163.8 mg, 0.33 mmol) in CH₂Cl₂ (4 mL), Dess-Martin periodinane (284.3 mg, 0.67 mmol) was added. The reaction was stirred at 22° C. for 30 min and then directly subject to flash column chromatography (acetone/hexanes=1:4) to yield compound 9c (137.6 g, 84%) as a pale-yellow oil. 9c: R_{f}=0.60 (silica, EtOAc:hexanes 1:1); [α]25 D=−4 (c=1.0 in MeOH); ¹H NMR (500 MHz, CDCl₃): δ=9.61-9.85 (m, 1H), 5.04 (brs, 1H, NH), 4.14 (brs, 1H), 2.95-3.28 (m, 4H), 2.29-2.51 (m, 2H), 1.16-1.80 (m, 37H) ppm; ¹³C NMR (126 MHz, CDCl₃): δ=202.6, 171.9, 155.6, 155.5, 82.0, 79.7, 79.4, 53.8, 46.9, 46.6, 43.9, 30.5, 28.6, 28.4, 28.1, 26.5, 24.4, 21.9 ppm; HRMS (m/z): [M+Na]⁺ calcd for C₂₅H₄₆N₂O₇Na⁺ 509.3197, found 509.3206.

### Synthesis of (1S,10aR)-1-aminodecahydropyrido[1,2-a]azepine-1-carboxylic acid (10)

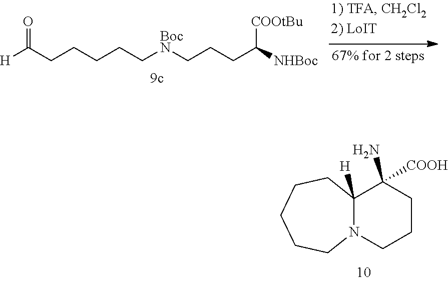

To a stirred solution of 9c (137.6 mg, 0.28 mmol) in CH₂Cl₂ (2 mL) at 0° C., TFA/H₂O (2.1 mL, v/v=20/1) was added. The reaction mixture was warmed up to 22° C. and kept at room temperature for 12 h. The solvent was removed in vacuo and the residue was dissolved in phosphate buffer(2.8 mL, 500 mM K₂HPO₃, pH=7.5) to make the substrate stock solution (100 mM calculated based on the starting material). The enzymatic reaction was set up in a 50 mL falcon tube by mixing the substrate stock solution with reaction buffer to yield a 20 mL final reaction mixture [50 mM K₂HPO₄, 50 μM PLP, 5 mM substrate, pH 7.5]. The reaction was then initiated by addition of stock solutions of LolT (300 μM) to a final concentration at 5 μM. This enzymatic reaction was then incubated at 28° C. with gentle shaking (150 rpm) for 12 hrs. The reaction was quenched with 2 ml of 1 M HCl, and the crude mixture was directly subject to ion exchange chromatography using CM-SEPHADEX resin (from SIGMA). The enzymatic product was eluted using 0.5 M NH₃·H₂O. After removing the solvent in vacuo, pure compound 10 was obtained as a pale-yellow oil (14.3 mg, 67%).10: R_{f}=0.40 (silica, CH₂Cl₂:MeOH:NH₃·H₂O 6:4:1.5); [α]23 D=+5 (c=1.0 in MeOH); ¹H NMR (500 MHz, CD₃CN): δ=3.22-3.31 (m, 1H), 3.02-3.14 (m, 2H), 2.93 (ddd, J=12.4, 12.4, 3.7 Hz, 1H), 2.57 (dd, J=8.8, 1.6 Hz, 1H), 2.29 (dd, J=15.3, 6.6 Hz, 1H), 1.99-2.12 (m, 1H), 1.86-1.99 (m, 1H), 1.79-1.86 (m, 1H), 1.66-1.79 (m, 4H), 1.31-1.53 (m, 4H) ppm; ¹³C NMR (126 MHz, CD₃CN): δ=178.1, 74.9, 58.6, 56.0, 55.9, 38.1, 28.8, 28.3, 24.5, 22.8, 22.7 ppm; HRMS (m/z): HRMS (m/z): [M+H]⁺ calcd for C₁₁H₂₁N₂O₂⁺ 213.1598, found 213.1603.

### Synthesis of tert-butyl N²,N⁶-bis(tert-butoxycarbonyl)-Ns-(5-((tert-butyldimethylsilyl)oxy)pentyl)-L-lysinate (10a)

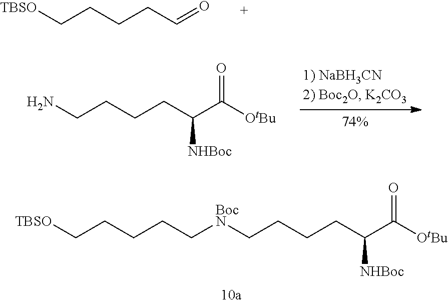 [SMILES: CC(C)(C)[Si](C)(C)OCCCCC=O ; CC(C)(C)OC(=O)N[C@@H](CCCCN)C(=O)OC(C)(C)C ; CC(C)(C)OC(=O)N[C@@H](CCCCN(CCCCCO[Si](C)(C)C(C)(C)C)C(=O)OC(C)(C)C)C(=O)OC(C)(C)C]

To a stirred solution of (S)-tert-butyl 6-amino-2-((tert-butoxycarbonyl)amino)hexanoate (195.0 mg, 0.645 mmol) in MeOH (26 mL) at 22° C., 5-((tert-butyldimethylsilyl)oxy)pentanal² (153.3 mg, 0.709 mmol), AcOH (57.7 mg, 55 μl, 0.97 mmol), and NaBH₃CN (121.5 mg, 1.94 mmol) were sequentially added. The resulting mixture was stirred at 22° C. for 8 h and concentrated in vacuo. The residue was dissolved in THF/H₂O (6.6 ml, v/v=10/1). To this solution, K₂CO₃ (356.6 mg, 2.58 mmol) and Boc₂O (563.0 g, 2.58 mmol) were added and stirred for 4 h at 22° C. Solvent was removed under in vacuo and the residue was subject to flash column chromatography and eluted with acetone/hexanes (1:10) to yield compound 10a (286.8 mg, 74% for the two steps) as a pale-yellow oil. 5a: R_{f}=0.46 (silica, acetone:hexanes 1:4); [α]23 D=−4 (c=1.0 in MeOH); ¹H NMR (500 MHz, CDCl₃): δ=5.06/4.99 (brs, 1H, NH), 4.10 (brs, 1H), 3.55 (dd, J=6.5, 6.5 Hz, 2H), 3.10 (brs, 4H), 1.80-1.66 (m, 1H), 1.66-1.34 (m, 34H), 1.34-1.13 (m, 4H), 0.84 (s, 9H), 0.00 (s, 6H) ppm; ¹³C NMR (126 MHz, CDCl₃): δ=172.0, 155.7, 155.5, 81.7, 79.5, 79.1, 63.1, 54.0, 47.1, 46.9, 46.5, 32.7, 28.6, 28.4, 28.1, 26.0, 23.2, 22.6, 18.4, −5.2 ppm; HRMS (m/z): [M+Na]⁺ calcd for C₃₁H₆₂N₂O₇SiNa⁺ 625.4218, found 625.4224.

### Synthesis of tert-butyl N²,N⁶-bis(tert-butoxycarbonyl)-N⁶-(5-hydroxypentyl)-L-lysinate (10b)

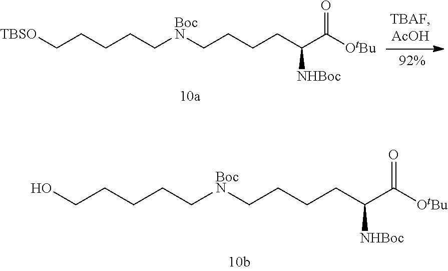 [SMILES: CC(C)(C)OC(=O)N[C@@H](CCCCN(CCCCCO[Si](C)(C)C(C)(C)C)C(=O)OC(C)(C)C)C(=O)OC(C)(C)C ; CC(C)(C)OC(=O)N[C@@H](CCCCN(CCCCCO)C(=O)OC(C)(C)C)C(=O)OC(C)(C)C]

To a stirred solution of 10a (286.8 mg, 0.476 mmol) in THF (5 mL), TBAF (1.5 mL, 1.5 mmol) and AcOH (94.4 mg, 90 μl, 1.5 mmol) were sequentially at 22° C. The resulting mixture was stirred at 22° C. for 8 h and then the solvent was removed in vacuo. The residue was subject to flash column chromatography (acetone/hexanes=1:2) to yield compound 10b (214.5 mg, 92%) as a pale-yellow oil. 10b: R_{f}=0.62 (silica, acetone:hexanes 1:2); [α]23 D=−5 (c=2.0 in MeOH); ¹H NMR (500 MHz, CDCl₃): δ=5.10/5.03 (brs, 1H, NH), 4.08 (brs, 1H), 3.65-3.45 (m, 2H), 3.09 (brs, 4H), 2.29 (s, 1H, OH), 1.82-1.08 (m, 39H) ppm; ¹³C NMR (126 MHz, CDCl₃): δ=172.0, 155.7, 155.5, 81.7, 79.6, 79.2, 62.5, 53.9, 46.9, 32.6, 32.4, 28.5, 28.4, 28.0, 23.0, 22.5 ppm; HRMS (m/z): [M+H]⁺ calcd for C₂₅H₄₈N₂O₇⁺ 489.3534, found 489.3540.

### Synthesis of tert-butyl N²,N⁶-bis(tert-butoxycarbonyl)-N⁶-(5-oxopentyl)-L-lysinate (10c)

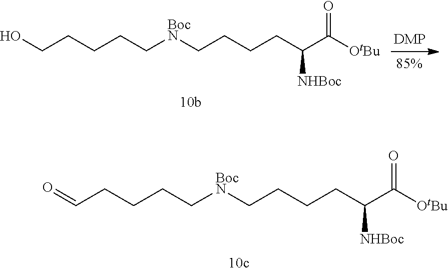 [SMILES: CC(C)(C)OC(=O)N[C@@H](CCCCN(CCCCCO)C(=O)OC(C)(C)C)C(=O)OC(C)(C)C ; CC(C)(C)OC(=O)N[C@@H](CCCCN(CCCCC=O)C(=O)OC(C)(C)C)C(=O)OC(C)(C)C]

To a stirred solution of compound 10b (214.5 mg, 0.44 mmol) in CH₂Cl₂ (5 mL) at 22° C., Dess-Martin periodinane (372.3 mg, 0.88 mmol) was. The reaction was kept at room temperature for 30 min and then directly subject to flash column chromatography (acetone/hexanes=1:4) to yield compound 10c (182.1 mg, 85%) as a pale-yellow oil. 6c: R_{f}=0.56 (silica, acetone:hexanes 1:2); [α]23 D=−8 (c=1.0 in MeOH); ¹H NMR (500 MHz, CDCl₃): δ=9.72 (s, 1H), 5.06/5.02 (brs, 1H, NH), 4.09 (brs, 1H), 3.11 (brs, 4H), 2.42 (dd, J=7.0, 7.0 Hz, 2H), 1.82-1.65 (m, 1H), 1.65-1.13 (m, 36H) ppm; ¹³C NMR (126 MHz, CDCl₃): δ=202.3, 202.0, 171.9, 155.5, 155.4, 81.7, 79.5, 79.3, 53.9, 46.5, 43.5, 32.5, 28.4, 28.3, 28.0, 22.4, 19.2 ppm; HRMS (m/z): [M+Na]⁺ calcd for C₂₅H₄₇N₂O₇Na⁺ 509.3198, found 509.3203.

### Synthesis of tert-butyl N²,N⁶-bis(tert-butoxycarbonyl)-N-(6-((tert-butyldimethylsilyl)oxy)hexyl)-L-lysinate (11a)

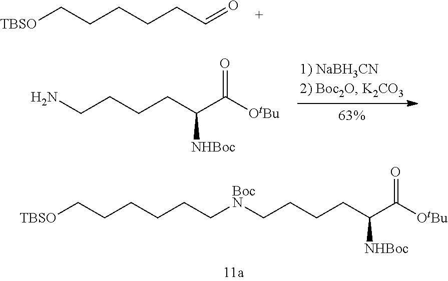

To a stirred solution of (S)-tert-butyl 6-amino-2-((tert-butoxycarbonyl)amino)hexanoate (200.0 mg, 0.66 mmol) in MeOH (27 mL) at 22° C., 6-((tert-butyldimethylsilyl)oxy)hexanal² (168.0 mg, 0.73 mmol), AcOH (118.5 mg, 113 μl, 1.97 mmol), and NaBH₃CN (124.3 mg, 1.98 mmol) were sequentially added. The resulting mixture was stirred at 22° C. for 8 h and concentrated in vacuo. The residue was dissolved in THF/H₂O (6.6 ml, v/v=10/1). To this solution, K₂CO₃ (356.6 mg, 2.58 mmol) and Boc₂O (563.0 g, 2.58 mmol) were added and stirred for 4 h at 22° C. Solvent was removed under in vacuo and the residue was subject to flash column chromatography and eluted with acetone/hexanes (1:10) to yield compound 11a (257.1 mg, 63% for the two steps) as a pale-yellow oil. 5a: R_{f}=0.60 (silica, acetone:hexanes 1:4); [α]23 D=−5 (c=1.0 in MeOH); ¹H NMR (500 MHz, CDCl₃): δ=5.05 (brs, 1H, NH), 4.25-3.90 (m, 1H), 3.59 (dd, J=6.6, 6.6 Hz, 2H), 3.31-2.92 (m, 4H), 1.82-1.37 (m, 35H), 1.37-1.14 (m, 6H), 0.88 (s, 9H), 0.03 (s, 6H) ppm; ¹³C NMR (126 MHz, CDCl₃): δ=172.1, 155.8, 155.5, 81.8, 79.7, 79.2, 63.3, 54.1, 47.2, 46.8, 33.0, 32.8, 28.6, 28.5, 28.2, 26.9, 26.1, 25.8, 22.6, 18.5, −5.1 ppm; HRMS (m/z): [M+H]⁺ calcd for C₃₂H₆₄N₂O₇SiNa⁺ 639.9452, found 639.4380.

### Synthesis of tert-butyl N²,N⁶-bis(tert-butoxycarbonyl)-N⁶-(6-hydroxyhexyl)-L-lysinate (10b)

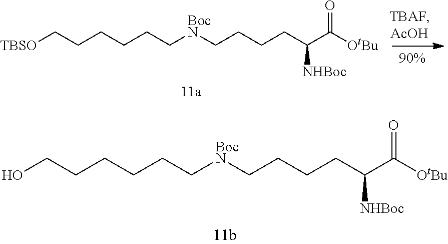 [SMILES: CC(C)(C)OC(=O)N[C@@H](CCCCN(CCCCCCO[Si](C)(C)C(C)(C)C)C(=O)OC(C)(C)C)C(=O)OC(C)(C)C ; CC(C)(C)OC(=O)N[C@@H](CCCCN(CCCCCCO)C(=O)OC(C)(C)C)C(=O)OC(C)(C)C]

To a stirred solution of 10a (257.1 mg, 0.42 mmol) in THF (5 mL), TBAF (1.5 mL, 1.5 mmol) and AcOH (94.4 mg, 90 μl, 1.5 mmol) were sequentially at 22° C. The resulting mixture was stirred at 22° C. for 8 h and then the solvent was removed in vacuo. The residue was subject to flash column chromatography (acetone/hexanes=1:2) to yield compound 10b (188.2 mg, 90%) as a pale-yellow oil. 10b: R_{f}=0.45 (silica, acetone:hexanes 1:2); [α]23 D=−5 (c=1.0 in MeOH); ¹H NMR (500 MHz, CDCl₃): δ=5.05 (brs, 1H, NH), 4.13 (brs, 1H), 3.62 (dd, J=6.5, 6.5 Hz, 2H), 3.29-2.90 (m, 4H), 1.75 (s, 2H), 1.65-1.15 (m, 40H) ppm; ¹³C NMR (126 MHz, CDCl₃): δ=172.1, 155.8, 155.5, 81.8, 79.7, 79.3, 62.8, 54.0, 46.8, 32.8, 28.6, 28.5, 28.13, 26.6, 25.5, 22.6 ppm; HRMS (m/z): [M+H]⁺ calcd for C₂₆H₅₁N₂O₇⁺ 503.3691, found 503.3696.

### Synthesis of tert-butyl N²,N⁶-bis(tert-butoxycarbonyl)-N⁶-(6-oxohexyl)-L-lysinate (11 c)

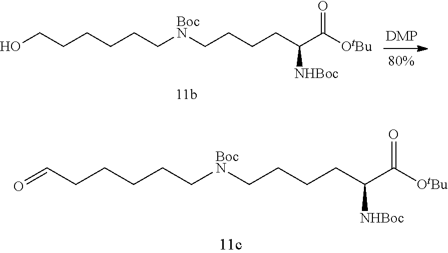 [SMILES: CC(C)(C)OC(=O)N[C@@H](CCCCN(CCCCCCO)C(=O)OC(C)(C)C)C(=O)OC(C)(C)C ; CC(C)(C)OC(=O)N[C@@H](CCCCN(CCCCCC=O)C(=O)OC(C)(C)C)C(=O)OC(C)(C)C]

To a stirred solution of compound 10b (188.2 mg, 0.37 mmol) in CH₂Cl₂ (4 mL) at 22° C., Dess-Martin periodinane (317.6 mg, 0.75 mmol) was added. The reaction was kept at room temperature for 30 min and then directly subject to flash column chromatography (acetone/hexanes=1:4) to yield compound 10c (150.4 mg, 80%) as a pale-yellow oil. 6c: R_{f}=0.56 (silica, acetone:hexanes 1:2); [α]23 D=−6 (c=1.0 in MeOH); ¹H NMR (500 MHz, CDCl₃): δ=9.75 (s, 1H), 5.05 (brs, 1H, NH), 4.13 (brs, 1H), 3.12 (brs, 4H), 2.42 (t, J=7.4 Hz, 2H), 1.89-1.69 (m, 2H), 1.69-1.54 (m, 3H), 1.54-1.35 (m, 30H), 1.35-1.12 (m, 4H) ppm; ¹³C NMR (126 MHz, CDCl₃): δ=202.6, 172.1, 155.7, 155.5, 81.8, 79.7, 79.3, 54.0, 46.9, 43.9, 32.7, 28.6, 28.5, 28.1, 26.5, 22.6, 21.9 ppm; HRMS (m/z): [M+Na]⁺ calcd for C₂₆H₄₉N₂O₇Na⁺ 523.3354, found 523.3359.

### Synthesis of tert-butyl (3-((tert-butoxycarbonyl)amino)propyl)(4-hydroxybutyl)carbamate (12a)

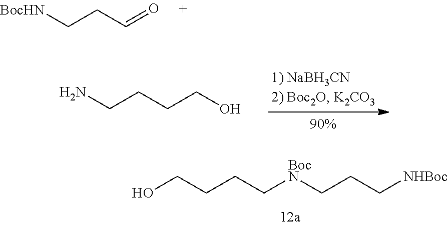 [SMILES: CC(C)(C)OC(=O)NCCC=O ; NCCCCO ; CC(C)(C)OC(=O)NCCCN(CCCCO)C(=O)OC(C)(C)C]

To a stirred solution of tert-Butyl (3-oxopropyl)carbamate (450.0 mg, 2.6 mmol) in MeOH (27 mL) at 22° C., 4-Amino-1-butanol² (926.0 mg, 10.4 mmol), AcOH (624.5 mg, 595 μl, 10.4 mmol), and NaBH₃CN (489.8 mg, 7.8 mmol) were sequentially added. The resulting mixture was stirred at 22° C. for 8 h and concentrated in vacuo. The residue was subjected to flash column chromatography using DCM/MeOH (10:1) as the mobile phase to yield a yellow oil product, R_{f}=0.68 (silica, DCM:MeOH 4:1). The product was dissolved in THF/H₂O (11 ml, v/v=10/1). To the stirred solution was added K₂CO₃ (1.0780 g, 7.8 mmol) and Boc₂O (1.1350 g, 5.2 mmol), and stirred at 22° C. for 30 mins. The reaction was concentrated in vacuo and the residue was subject to flash column chromatography (acetone/hexanes=1:4) to yield compound 12a (810.8 mg, 90% for two steps) as a pale-yellow oil. 7a: R_{f}=0.55 (silica, acetone:hexanes 1:2); ¹H NMR (500 MHz, CDCl₃): δ=5.27/4.77 (brs, 1H, NH), 3.61 (dd, J=6.3, 6.3 Hz, 2H), 2.87-3.36 (m, 6H), 2.40 (brs, 1H, OH), 1.45-1.82 (m, 6H), 1.25-1.45 (m, 18H) ppm; ¹³C NMR (126 MHz, CDCl₃): δ=156.2, 79.7, 79.2, 62.4, 46.9, 44.5, 44.3, 37.8, 29.8, 29.0, 28.5, 25.0, 24.7 ppm; HRMS (m/z): [M+H]⁺ calcd for C₁₇H₃₄N₂O₅⁺ 347.3, found 347.4.

### Synthesis of tert-butyl (3-((tert-butoxycarbonyl)amino)propyl)(4-oxobutyl)carbamate (12b)

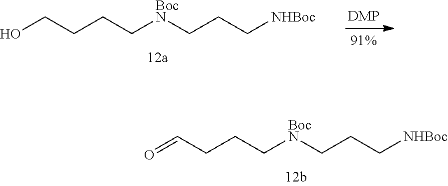 [SMILES: CC(C)(C)OC(=O)NCCCN(CCCCO)C(=O)OC(C)(C)C ; CC(C)(C)OC(=O)NCCCN(CCCC=O)C(=O)OC(C)(C)C]

To a stirred solution of compound 12a (810.8 mg, 2.34 mmol) in CH₂Cl₂ (10 mL) at 22° C., Dess-Martin periodinane (1.1148 g, 3.51 mmol) was added. The reaction was kept at room temperature for 30 min and then directly subject to flash column chromatography (acetone/hexanes=1:10) to yield compound 12c (733.4 mg, 91%) as a pale-yellow oil. 12b: R_{f}=0.65 (silica, acetone:hexanes 1:2); ¹H NMR (500 MHz, CDCl₃): δ=9.66 (s, 1H), 5.25/4.82 (brs, 1H, NH), 2.90-3.17 (m, 6H), 2.35 (dd, J=7.7, 7.7 Hz, 2H), 1.67-1.80 (m, 2H), 1.56 (brs, 2H), 1.18-1.44 (m, 18H) ppm; ¹³C NMR (126 MHz, CDCl₃): δ=201.6, 201.3, 156.0, 155.5, 79.7, 78.8, 50.3, 45.9, 44.3, 43.6, 40.8, 37.4, 28.8, 28.3, 28.3, 20.9, 20.6 ppm; HRMS (m/z): [M+H]⁺ calcd for C₁₇H₃₂N₂O₅⁺ 345.2, found 345.3.

What is claimed is:

1. A method for yielding an azacyclic α-amino acid, comprising:
providing a Mannich cyclase in a solution; and
contacting the Mannich cyclase with a substrate.

2. The method of claim 1, wherein the Mannich cyclase is LolT.

3. The method of claim 1, wherein the solution comprises a culture of live organisms heterologously expressing the Mannich cyclase.

4. The method of claim 1, wherein the solution comprises enriched Mannich cyclase that purified from organisms that heterologously expressed the Mannich cyclase; or wherein the solution comprises an organismal lysate of organisms that heterologously expressed the Mannich cyclase.

5. The method of claim 4 further comprising:
expressing the Mannich cyclase in the organisms, wherein the expression of Mannich cyclase is heterologous; and
lysing the organisms to generate a lysate.

6. The method of claim 5 further comprising:
enriching the Mannich cyclase from the lysate.

7. The method of claim 1, wherein the substrate is selected from Substrate A, Substrate B, or Substrate C.

8. The method of claim 7, wherein the substrate is Substrate A; wherein n is 1, 2, or 3; wherein m is 1, 2, or 3

9. The method of claim 8, wherein when n is 1, m is 1, 2 or 3, when n is 2 or 3, m is 1 or 2, when m is 1 or 2, n is 1, 2, or 3, and when m is 3, n is 1.

10. The method of claim 8, wherein either n or m is not 1.

11. The method of claim 7, wherein the substrate is Substrate B; wherein n is 1, 2, or 3; wherein m is 1, 2, or 3

12. The method of claim 11, wherein when n is 1, m is 1, 2 or 3, when n is 2 or 3, m is 1 or 2, when m is 1 or 2, n is 1, 2, or 3, and when m is 3, n is 1.

13. The method of claim 11 further comprising:
deprotecting the substrate.

14. The method of claim 7, wherein the substrate is Substrate C; wherein n is 1 or 2.

15. The method of claim 14, wherein when n is 1, R is an alkyl, an alkenyl, a phenyl, a phenethyl, a naphthalenyl, or a pyridinyl.

16. The method of claim 14, wherein when n is 2, R is an alkyl or an alkenyl.

17. The method of claim 1 further comprising:
enriching a product generated from the substrate via the Mannich cyclase.

18. An organism, comprising:
a nucleic acid comprising a sequence for heterologous expression a Mannich cyclase, wherein the Mannich cyclase is LolT.

19. The organism of claim 18, wherein the organism is E. coli.

20. The organism of claim 19, wherein the nucleic acid comprises SEQ ID NO: 1.


SEQUENCE LISTING (2 sequences)

SEQ ID NO: 1 - DNA, 1425 bp, Penicillium expansum
  source 1..1425: mol_type = other DNA
atgcccgcccttccactgtcggaaaatgaaggttggaaacgtccaactacgccattcggaaagccgatgcttaagcatttctgtatgaatccggaataccggaatttaaatgccccgtcatgtggcagctggccaaagaccgtacgcgaccaatggcgtagatatttagacgaccttgaagcacaacctgattatttcagcgaagtgaaacaaggacccgttatacaggaagcgcgacgggaagttgcacagctcctgcacgcaagagtgagcgagtgcgttttcatcagcaacgctacgactggaatctacacagttttgcataacatccctttcgataaggatgacgtgatcattacgttttcaactacatacggagctattgacaacgctattgcaagtatggcagagactcaacctttccaaacacgcaaagtaacggtcgatttaccgatgcgcggagaagacatcgtcgcaagattcgaggggatggtggcacagattaaagccgagggcctgcaccctcgccttgctgtccttgagacgattgtttcgatccccgccatacgcatgccttttgagagcctggtacaggcatgccaaagagagggagtactgagtttggtggacggcgctcactctatcggacagttttccttaaatcttgaggttcttcagcctgacttcttcatcatggattgtcacaaatggcttttcgtgccacggccttgcgcagccctttatgttccagagcgaaatcaacattatattagatcaactattccgccgtcattcggtttcataccccgggacggcaagccggccctgcccctgtggtctaagcagtctggtggtgggtcatccggttcaacagctacagactttgagaccatcttcgcatacgtcgcaacgtctgataatatgccgcacatgtgtattcctacagcgcttaagttccggcgggaagtgtgcggtggtgaagaggctatctaccagtatttgcgcgtgttagccaaagagggcggcgatagagtagcggcaatattgggcactgaagtattagacgagaagccggccggagagtataagagccaacgcacaccttcggagatgcgcgactgcggaatagccacagtgcgcttacctctcgctgtgagctcatccttaaagccgccaccgcattctggtaccccttacagcccgcttagtgacgaagaggtaggtccggccgtgcattacctctcgatgaccttggcagaaacacacaaaacttggctcccattgatcgatcacggtggttatatttgggtacgcctttgcgcccaaatatatttggacacgtcagacttcgagtggatcggcaacgtgttgaaggaaatatgcgagacaataggaaagaagggacacgtcatcagtaagcactaa

SEQ ID NO: 2 - DNA, 1260 bp, Epichloe uncinata
  source 1..1260: mol_type = other DNA
atggctacagttgtaagggaggcatttgaaaaccacgtcaagttggtagagtctcgtaatagcccgggtcatgttctcgcgtcgtctgaagctagcttctttgttgcggacctgaacgacatcgtccgtaagtgggcagcgtggaaaaaagcccttccggatgttaccccgttttttgcggtgaagtccagctatgatcgtcgcctgatccagactttggcgacgtgcggtgctggttttgactgcgcatctgtagaggaaattgagctgattctgagcttaggcatcggcgcggagcgcattgtgtttacccatccgtgtaaaccggtgagctcattgggcctgtgtcgtaagctgggcatcaccctgatcacgttcgacaacgagtgcgaactgcgcaagctgcatcaccactacccggaagcacagacggtgctgcgcgttttcgctgacgatccgaccaatgcagaccctctgggcacgaaattcggcgcggctcgtgaagatatcgacggtttagttcgcttggtcaaagaattgaacatgaaattggcgggtgcctcgttccacgcagctccgagcgtggcggtggatgcagcagcctacgtgaggggcatccgcgacgcggcagaggtattcgcgcgtgcacgtcgtgtcggtctgaacccgaccgtgctggacatcggcggtggctataccgatagcaccttccagcaaattgccggtgcggttcgtccggcaatcgcggagtgctttaaaagccaggttgtcgagggtcgtctgcgcattctggcggagccaggcaccctgtttagctgcagcccgttctatctggcggtgaaggtggtggcgcgtcgtcgcaacgccgcggcgtttggtaatgagccagcaactcgtttgtacatcaacgacggcatctacagcaactttatgatgcgttttatcgtgaacatgaccttctccccggttgcggttattcgtaagggtgtttggtatgaccagaccgaacaaacgatgcgcagagaagcttgttccctgtggggtcgtagttgcgattctaacgactgcattaatcgtgattgccgtctggacccggaggttggcgttggtgattggcttgtgttcaaggatatgggtgcgtacaccaccgtttgtaataccaccttcaacggtttcaccagctccaatcacaccatttacattgaaccgactcaggttgataaagcccaatccaccttcgagcaactggaactcgcgatt